(19) United States
(12) Patent Application Publication
(10) Pub. No.: US 2022/0401579 A1
(43) Pub. Date: Dec. 22, 2022

(54) INCLUSION COMPLEXES OF PHARMACEUTICALS AND CYCLIC OLIGOMERS

(71) Applicants: DISPERSOL TECHNOLOGIES, LLC, Georgetown, TX (US); BOARD OF REGENTS, THE UNIVERSITY OF TEXAS SYSTEM, Austin, TX (US)

(72) Inventors: Robert O. WILLIAMS, III, Austin, TX (US); Urvi GALA, Austin, TX (US); Dave MILLER, Georgetown, TX (US); Angela SPANGENBERG, Austin, TX (US); Daniel J. ELLENBERGER, Cedar Park, TX (US)

(73) Assignees: DISPERSOL TECHNOLOGIES, LLC, Georgetown, TX (US); BOARD OF REGENTS, THE UNIVERSITY OF TEXAS SYSTEM, Austin, TX (US)

(21) Appl. No.: 17/777,102

(22) PCT Filed: Sep. 24, 2020
(86) PCT No.: PCT/US2020/052418

§ 371 Date: May 16, 2022

Related U.S. Application Data
(60) Provisional application No. 62/942,105, filed on Nov. 30, 2019.
(60) Provisional application No. 62/942,107, filed on Nov. 30, 2019.

(51) Int. Cl. A61K 47/69; A61K 31/496; A61K 31/44; A61K 31/5377

(52) U.S. Cl. CPC A61K 47/6951; A61K 31/496; A61K 31/44; A61K 31/5377

(57) ABSTRACT

Pharmaceutical formulations including an inclusion complex of an API with a cyclic oligomer, and methods of forming such pharmaceutical formulations are described.

### RELATED APPLICATIONS

This application claims priority to U.S. Provisional Patent Application No. 62/942,105, filed Nov. 30, 2019, and to U.S. Provisional Patent Application No. 62/942,107, filed Nov. 30, 2019, both of which are incorporated by reference in their entireties.

### TECHNICAL FIELD

The present disclosure relates to pharmaceutical formulations including inclusion complexes of active pharmaceutical ingredients (APIs) and cyclic oligomers and methods of forming such inclusion complexes and pharmaceutical formulations.

### BACKGROUND

Many APIs have poor solubility in aqueous solutions and poor bioavailability in patients. While existing processes of formulating APIs for administration to patients fail to provide adequate solutions to these problems, development of new methods of formulating APIs in order to increase solubility and bioavailability is challenging.

### SUMMARY

The present disclosure provides a pharmaceutical formulation. The pharmaceutical formulation comprises an inclusion complex; an active pharmaceutical ingredient (API), or a pharmaceutically acceptable salt, ester, derivative, analog, prodrug, hydrate, or solvate thereof, or any combination thereof, wherein the API is not abiraterone or a pharmaceutically acceptable salt, ester, derivative, analog, prodrug, hydrate, or solvate thereof; and a cyclic oligomer. At least a portion of the API is present in the inclusion complex with the cyclic oligomer. The pharmaceutical formulation is formed by a method comprising: thermokinetically processing the API and the cyclic oligomer for less than 300 seconds to form an inclusion complex of the API and the cyclic oligomer.

According to various further embodiments of the pharmaceutical formulation, which may all be combined with one another unless clearly mutually exclusive:

(i) at least 90%, 91%, 92%, 93%, 94%, 95%, 96%, 97%, 98%, 99%, or 99.9% of the API may be present in the inclusion complex with the cyclic oligomer.

(ii) a molecule of the API, or the pharmaceutically acceptable salt, ester, derivative, analog, prodrug, hydrate, or solvate thereof, or any combination thereof, may be sized to allow inclusion of at least 10%, 20%, 30%, 40%, 50%, 60%, 70%, 80%, 90% or 99.9% of the molecule of the API within a central cavity of the cyclic oligomer.

(iii) a molecule of the API, or the pharmaceutically acceptable salt, ester, derivative, analog, prodrug, hydrate, or solvate thereof, or any combinations thereof, may have at least a minor axis diameter smaller than a diameter of a central cavity of the cyclic oligomer.

(iv) the diameter may be a kinetic diameter.

(v) the diameter of a central cavity of the cyclic oligomer may be from 4 Å to 12 Å.

(vi) the diameter of a central cavity of the cyclic oligomer may be up to 5, 6, 7, 8, 9, 10, 11, or 12 Å.

(vii) the cyclic oligomer may be an α-cyclodextrin, or a derivative thereof; and the API, or the pharmaceutically acceptable salt, ester, derivative, analog, prodrug, hydrate, or solvate thereof, or any combinations thereof, may have a minor axis diameter up to 4.7-5.3 Å.

(viii) the cyclic oligomer may be a β-cyclodextrin, or a derivative thereof; and a molecule of the API, or the pharmaceutically acceptable salt, ester, derivative, analog, prodrug, hydrate, or solvate thereof, or any combinations thereof, may have a minor axis diameter up to 6.0-6.5 Å.

(ix) the cyclic oligomer may be a γ-cyclodextrin, or a derivative thereof; and a molecule of the API, or the pharmaceutically acceptable salt, ester, derivative, analog, prodrug, hydrate, or solvate thereof, or any combinations thereof, may have a minor axis diameter up to 7.5-8.3 Å.

(x) the cyclic oligomer may be a 6-cyclodextrin, or a derivative thereof; and a molecule of the API, or the pharmaceutically acceptable salt, ester, derivative, analog, prodrug, hydrate, or solvate thereof, or any combinations thereof, may have a minor axis diameter up to 10.3-11.2 Å.

(xi) the API may be selected from: itraconazole (ITZ), sorafenib (SOR), rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, ziprasidone, a pharmaceutically acceptable salt, ester, derivative, analog, prodrug, hydrate, or solvate thereof, and any combinations thereof.

(xii) the API and the cyclic oligomer may be present in the pharmaceutical formulation in a molar ratio of API:cyclic oligomer from 1:0.25 to 1:25.

(xiii) the API and the cyclic oligomer may be present in the pharmaceutical formulation in a molar ratio of API:cyclic oligomer from 1:1 to 1:3.

(xiv) the API may comprise less than 10%, 9%, 8%, 7%, 6%, 5%, 4%, 3%, 2%, 1%, or 0.1% crystalline API.

(xv) in response to heating the pharmaceutical formulation to a temperature up to 90% of the melting point of a crystalline form of the API, and allowing the pharmaceutical formulation to cool to room temperature, less than 10%, 9%, 8%, 7%, 6%, 5%, 4%, 3%, 2%, 1%, or 0.1% of the API may be in crystalline form.

(xvi) the less than 10%, 9%, 8%, 7%, 6%, 5%, 4%, 3%, 2%, 1%, or 0.1% of the API in crystalline form may be determined by a method comprising X-ray diffraction (XRD).

(xvii) the API may comprise less than 10%, 9%, 8%, 7%, 6%, 5%, 4%, 3%, 2%, 1%, or 0.1% crystalline API as determined by a method comprising X-ray diffraction (XRD), modulated differential scanning calorimetry (mDSC), Raman spectroscopy, solid-state Nuclear Magnetic Resonance (NMR) spectroscopy, or any combination thereof.

(xviii) the pharmaceutical formulation may comprise 1% to 50% by weight of the API.

(xix) the pharmaceutical formulation may comprise at least 10% by weight of the API.

(xx) the cyclic oligomer may comprise a cyclic oligosaccharide or cyclic oligosaccharide derivative.

(xxi) the cyclic oligosaccharide or cyclic oligosaccharide derivative may comprise a cyclodextrin or a cyclodextrin derivative.

(xxii) the cyclodextrin derivative may comprise a hydroxy propyl β cyclodextrin.

(xxiii) the cyclodextrin derivative may comprise a sodium (Na) sulfo-butyl ether β cyclodextrin.

(xxiv) the cyclodextrin derivative may comprise a sulfobutylether functional group.

(xxv) the cyclodextrin derivative may comprise a methyl group.

(xxvi) the pharmaceutical formulation may comprise 50% to 99% by weight of the cyclic oligomer.

(xxvii) the pharmaceutical formulation may comprise at least 60% by weight of the cyclic oligomer.

(xxviii) the pharmaceutical formulation may provide an increase in an Area Under the Drug Dissolution versus time Curve (AUDC), Cₘₐₓ, or both, as compared to a formulation of an equivalent amount of the API and the cyclic oligomer prepared without thermokinetically processing the API and the cyclic oligomer, when the pharmaceutical formulation is analyzed using an in vitro dissolution assay.

(xxix) the AUDC, Cₘₐₓ, or both, may be analyzed by a method comprising HPLC analysis, UV spectrophotometry, or both.

(xxx) thermokinetically processing the API and the cyclic oligomer may be at a temperature less than or equal to 300° C.

The present disclosure also provides a method of making a pharmaceutical formulation. The method comprises: processing by thermokinetic compounding for less than 300 seconds (i) one or more API described herein, or a pharmaceutically acceptable salt, ester, derivative, analog, prodrug, hydrate, or solvate thereof, or any combination thereof; and (ii) one or more cyclic oligomer described herein; to form an inclusion complex described herein.

According to various further embodiments of the method, which may all be combined with one another unless clearly mutually exclusive:

(i) the processing by thermokinetic compounding may not cause substantial thermal degradation of the API.

(ii) the processing by thermokinetic compounding may not cause substantial thermal degradation of the cyclic oligomer.

(iii) the processing by thermokinetic compounding may be solvent-free.

### BRIEF DESCRIPTION OF THE DRAWINGS

The present disclosure may be further understood through reference to the attached figures in combination with the detailed description that follows.

FIG. 1 is a Table of example lots of Itraconazole (ITZ) and hydroxypropyl β cyclodextrin (HPBCD) compositions.

FIG. 2 is a Table of example thermokinetic compounding processing parameters for the example lots of Itraconazole (ITZ) and hydroxypropyl β cyclodextrin (HPBCD) compositions.

FIG. 3A is a graph reporting results of example thermokinetic compounding profiles for lot ITZ-004, which was thermokinetically compounded with a ratio of 1:2 ITZ:HPBCD.

FIG. 3B is a graph reporting results of example thermokinetic compounding profiles for lot ITZ-005, which was thermokinetically compounded with a ratio of 1:1 ITZ:HPBCD.

FIG. 3C is a graph reporting results of example thermokinetic compounding profiles for lot ITZ-006, which was thermokinetically compounded with a ratio of 2:1 ITZ:HPBCD.

FIG. 4 is a graph reporting example X-ray diffraction (XRD) results for lots ITZ-004, ITZ-005 and ITZ-006, as well as ITZ API.

FIG. 5 is a graph reporting example modulated differential scanning calorimetry (mDSC) results for lots ITZ-004, ITZ-005 and ITZ-006, as well as ITZ API.

FIG. 6A is a graph reporting example XRD results for ITZ-004, with or without heating of the sample for 15 hours at 90° C. prior to XRD analysis.

FIG. 6B is a graph reporting example XRD results for ITZ-005, with or without heating of the sample for 15 hours at 90° C. prior to XRD analysis.

FIG. 6C is a graph reporting example XRD results for ITZ-006, with or without heating of the sample for 15 hours at 90° C. prior to XRD analysis.

FIG. 7 is a graph reporting example XRD results for amorphous ITZ that was prepared by melt-quenching.

FIG. 8 is a graph reporting example Raman spectra of crystalline ITZ, HPBCD, amorphous ITZ, and lot ITZ-004 (1:2 ITZ:HPBCD).

FIG. 9 is a Table summarizing results of Raman spectral shift for crystalline ITZ, amorphous ITZ, and ITZ-004 (1:2 ITZ:HPBCD).

FIG. 10 is a graph reporting example detail of Raman spectra of crystalline ITZ, HPBCD, amorphous ITZ, and lot ITZ-004 (1:2 ITZ:HPBCD) over a Raman shift range of 390-422 cm⁻¹.

FIG. 11 is a graph reporting example detail of Raman spectra of crystalline ITZ, HPBCD, amorphous ITZ, and lot ITZ-004 (1:2 ITZ:HPBCD) over a Raman shift range of 590-830 cm⁻¹.

FIG. 12 is a graph reporting example detail of Raman spectra of crystalline ITZ, HPBCD, amorphous ITZ, and lot ITZ-004 (1:2 ITZ:HPBCD) over a Raman shift range of 895-985 cm⁻¹.

FIG. 13 is a graph reporting example detail of Raman spectra of crystalline ITZ, HPBCD, amorphous ITZ, and lot ITZ-004 (1:2 ITZ:HPBCD) over a Raman shift range of 1030-1180 cm-1.

FIG. 14 is a graph reporting example detail of Raman spectra of crystalline ITZ, HPBCD, amorphous ITZ, and lot ITZ-004 (1:2 ITZ:HPBCD) over a Raman shift range of 1180-1300 cm-1.

FIG. 15 is a graph reporting example detail of Raman spectra of crystalline ITZ, HPBCD, amorphous ITZ, and lot ITZ-004 (1:2 ITZ:HPBCD) over a Raman shift range of 1355-1445 cm⁻¹.

FIG. 16 is a graph reporting example detail of Raman spectra of crystalline ITZ, HPBCD, amorphous ITZ, and lot ITZ-004 (1:2 ITZ:HPBCD) over a Raman shift range of 1540-1640 cm-1.

FIG. 17 is a graph reporting example detail of Raman spectra of crystalline ITZ, HPBCD, amorphous ITZ, and lot ITZ-004 (1:2 ITZ:HPBCD) over a Raman shift range of 3045-3105 cm⁻¹.

FIG. 18 is a Table of example lots of Sorafenib (SOR) and hydroxypropyl β cyclodextrin (HPBCD) compositions.

FIG. 19 is a Table of example thermokinetic compounding processing parameters for the example lots of Sorafenib (SOR) and hydroxypropyl β cyclodextrin (HPBCD) compositions.

FIG. 20A is a graph reporting results of example thermokinetic compounding profiles for lot 1:2 SOR/HPBCD, which was thermokinetically compounded with a ratio of 1:2 SOR:HPBCD.

FIG. 20B is a graph reporting results of example thermokinetic compounding profiles for lot 1:1 SOR/HPBCD, which was thermokinetically compounded with a ratio of 1:1 SOR:HPBCD.

FIG. 20C is a graph reporting results of example thermokinetic compounding profiles for lot 2:1 SOR/HPBCD, which was thermokinetically compounded with a ratio of 2:1 SOR:HPBCD.

FIG. 21 is a graph reporting example X-ray diffraction (XRD) results for lots 1:2 SOR/HPBCD, 1:1 SOR/HPBCD and 2:1 SOR/HPBCD, as well as SOR API.

FIG. 22 is a graph reporting example modulated differential scanning calorimetry (mDSC) results for lots 1:2 SOR/HPBCD, 1:1 SOR/HPBCD and 2:1 SOR/HPBCD as well as SOR API.

FIG. 23A is a graph reporting example XRD results for 1:2 SOR/HPBCD, with or without heating of the sample for 6 hours at 150° C. prior to XRD analysis.

FIG. 23B is a graph reporting example XRD results for 1:1 SOR/HPBCD, with or without heating of the sample for 6 hours at 150° C. prior to XRD analysis.

FIG. 23C is a graph reporting example XRD results for 2:1 SOR/HPBCD, with or without heating of the sample for 6 hours at 150° C. prior to XRD analysis.

FIG. 24 is a graph reporting example XRD results for amorphous SOR that was prepared by melt-quenching.

FIG. 25 is a graph reporting example Raman spectra of crystalline SOR, HPBCD, amorphous SOR, and KPCs of 1:2 SOR/HPBCD and 1:1 SOR/HPBCD.

FIG. 26 is a Table summarizing results of Raman spectral shift for crystalline SOR, HPBCD, amorphous SOR, and KPCs of 1:2 SOR/HPBCD and 1:1 SOR/HPBCD.

FIG. 27 is a graph reporting example detail of Raman spectra of crystalline SOR, HPBCD, amorphous SOR, and KPCs of 1:2 SOR/HPBCD and 1:1 SOR/HPBCD over a Raman shift range of 300-360 cm⁻¹.

FIG. 28 is a graph reporting example detail of Raman spectra of crystalline SOR, HPBCD, amorphous SOR, and KPCs of 1:2 SOR/HPBCD and 1:1 SOR/HPBCD over a Raman shift range of 360-530 cm⁻¹.

FIG. 29 is a graph reporting example detail of Raman spectra of crystalline SOR, HPBCD, amorphous SOR, and KPCs of 1:2 SOR/HPBCD and 1:1 SOR/HPBCD over a Raman shift range of 635-745 cm⁻¹.

FIG. 30 is a graph reporting example detail of Raman spectra of crystalline SOR, HPBCD, amorphous SOR, and KPCs of 1:2 SOR/HPBCD and 1:1 SOR/HPBCD over a Raman shift range of 735-850 cm⁻¹.

FIG. 31 is a graph reporting example detail of Raman spectra of crystalline SOR, HPBCD, amorphous SOR, and KPCs of 1:2 SOR/HPBCD and 1:1 SOR/HPBCD over a Raman shift range of 850-980 cm⁻¹.

FIG. 32 is a graph reporting example detail of Raman spectra of crystalline SOR, HPBCD, amorphous SOR, and KPCs of 1:2 SOR/HPBCD and 1:1 SOR/HPBCD over a Raman shift range of 990-1050 cm⁻¹.

FIG. 33 is a graph reporting example detail of Raman spectra of crystalline SOR, HPBCD, amorphous SOR, and KPCs of 1:2 SOR/HPBCD and 1:1 SOR/HPBCD over a Raman shift range of 1095-1140 cm⁻¹.

FIG. 34 is a graph reporting example detail of Raman spectra of crystalline SOR, HPBCD, amorphous SOR, and KPCs of 1:2 SOR/HPBCD and 1:1 SOR/HPBCD over a Raman shift range of 1155-1210 cm⁻¹.

FIG. 35 is a graph reporting example detail of Raman spectra of crystalline SOR, HPBCD, amorphous SOR, and KPCs of 1:2 SOR/HPBCD and 1:1 SOR/HPBCD over a Raman shift range of 1240-1410 cm⁻¹.

FIG. 36 is a graph reporting example detail of Raman spectra of crystalline SOR, HPBCD, amorphous SOR, and KPCs of 1:2 SOR/HPBCD and 1:1 SOR/HPBCD over a Raman shift range of 1500-1760 cm⁻¹.

FIG. 37 is a graph reporting example detail of Raman spectra of crystalline SOR, HPBCD, amorphous SOR, and KPCs of 1:2 SOR/HPBCD and 1:1 SOR/HPBCD over a Raman shift range of 3030-3160 cm⁻¹.

FIG. 38 is a Table of example lots of Rivaroxaban (RIV) and hydroxypropyl R cyclodextrin (HPBCD) compositions.

FIG. 39 is a Table of example thermokinetic compounding processing parameters for the example lots of Rivaroxaban (RIV) and hydroxypropyl β cyclodextrin (HPBCD) compositions.

FIG. 40A is a graph reporting results of example thermokinetic compounding profiles for lot 1:2 RIV/HPBCD, which was thermokinetically compounded with a ratio of 1:2 RIV:HPBCD.

FIG. 40B is a graph reporting results of example thermokinetic compounding profiles for lot 1:1 RIV/HPBCD, which was thermokinetically compounded with a ratio of 1:1 RIV:HPBCD.

FIG. 40C is a graph reporting results of example thermokinetic compounding profiles for lot 2:1 RIV/HPBCD, which was thermokinetically compounded with a ratio of 2:1 RIV:HPBCD.

FIG. 41 is a graph reporting example X-ray diffraction (XRD) results for lots 1:2 RIV/HPBCD, 1:1 RIV/HPBCD and 2:1 RIV/HPBCD, as well as RIV API.

FIG. 42 is a graph reporting example modulated differential scanning calorimetry (mDSC) results for lots 1:2 RIV/HPBCD, 1:1 RIV/HPBCD and 2:1 RIV/HPBCD as well as RIV API.

FIG. 43A is a graph reporting example XRD results for 1:2 RIV/HPBCD, with or without heating of the sample for 6 hours at 150° C. prior to XRD analysis.

FIG. 43B is a graph reporting example XRD results for 1:1 RIV/HPBCD, with or without heating of the sample for 6 hours at 150° C. prior to XRD analysis.

FIG. 43C is a graph reporting example XRD results for 2:1 RIV/HPBCD, with or without heating of the sample for 6 hours at 150° C. prior to XRD analysis.

FIG. 44 is a graph reporting example XRD results for amorphous RIV that was prepared by melt-quenching.

FIG. 45 is a graph reporting example Raman spectra of crystalline RIV, HPBCD, amorphous RIV, and KPC of 1:2 RIV/HPBCD. Spectra shown are averages of three acquisitions.

FIG. 46 is a Table summarizing results of Raman spectral shift for crystalline RIV, HPBCD, amorphous RIV, and KPC of 1:2 SOR/HPBCD.

FIG. 47 is a graph reporting example detail of Raman spectra of crystalline RIV, HPBCD, amorphous RIV, and KPC of 1:2 RIV/HPBCD over a Raman shift range of 620-840 cm⁻¹.

FIG. 48 is a graph reporting example detail of Raman spectra of crystalline RIV, HPBCD, amorphous RIV, and KPC of 1:2 RIV/HPBCD over a Raman shift range of 1065-1145 cm⁻¹.

FIG. 49 is a graph reporting example detail of Raman spectra of crystalline RIV, HPBCD, amorphous RIV, and KPC of 1:2 RIV/HPBCD over a Raman shift range of 1395-1495 cm⁻¹.

FIG. 50 is a graph reporting example detail of Raman spectra of crystalline RIV, HPBCD, amorphous RIV, and KPC of 1:2 RIV/HPBCD over a Raman shift range of 1535-1630 cm⁻¹.

FIG. 51 is a schematic showing examples of cyclodextrin host molecule and API guest molecule of inclusion complexes having one host molecule and one guest molecule (left Panel), or two host molecules and one guest molecule (right Panel).

FIG. 52 shows the molecular structure of β cyclodextrin, in which R=CH₂CHOHCH₃ or H, having varying degrees of substitution at the 2, 3, and 6 positions.

FIG. 53 is a Table of example parameters, including central cavity diameter, in Angstroms (Å), of example cyclic oligomers, including α-cyclodextrin, β-cyclodextrin, γ-cyclodextrin and 6-cyclodextrin.

FIG. 54 is a Table of example lots of Nintedanib (NIN) and hydroxypropyl R cyclodextrin (HPBCD) compositions and example thermokinetic compounding processing parameters.

FIG. 55 is a graph reporting results of example thermokinetic compounding profiles for NIN lots 2, 3 and 4, which were thermokinetically compounded with ratios of 1:2, 1:1 and 2:1 NIN:HPBCD respectively.

FIG. 56 is a graph reporting example X-ray diffraction (XRD) results for NIN-HPBCD lots 2, 3 and 4.

FIG. 57 is a graph reporting example Raman spectra of crystalline NIN, 1:2, 1:1, and 2:1 physical mixtures, 1:2, 1:1, and 2:1 KPCs, and cryo-milled amorphous NIN.

FIG. 58 is a Table summarizing results of Raman spectral shift for crystalline NIN, 1:2, 1:1, and 2:1 physical mixtures, 1:2, 1:1, and 2:1 KPCs, and cryo-milled amorphous NIN.

FIG. 59 is a graph reporting example detail of Raman spectra of crystalline NIN, 1:2, 1:1, and 2:1 physical mixtures, 1:2, 1:1, and 2:1 KPCs, and cryo-milled amorphous NIN over a Raman shift range of 1420-1740 cm⁻¹.

FIG. 60 is a graph reporting example detail of Raman spectra of crystalline NIN, 1:2, 1:1, and 2:1 physical mixtures, 1:2, 1:1, and 2:1 KPCs, and cryo-milled amorphous NIN over a Raman shift range of 1080-1440 cm⁻¹.

FIG. 61 is a graph reporting example detail of Raman spectra of crystalline NIN, 1:2, 1:1, and 2:1 physical mixtures, 1:2, 1:1, and 2:1 KPCs, and cryo-milled amorphous NIN over a Raman shift range of 580-1040 cm⁻¹.

FIG. 62 is a graph reporting example data from in vitro dissolution analysis of ITZ-004 (1:2 ITZ:HPBCD) physical mixture (PM) and a KPC thereof.

FIG. 63 is a graph reporting example data from in vitro dissolution analysis of 1:2 RIV:HPBCD as a physical mixture (PM) and a KPC thereof.

FIG. 64 is a graph reporting example data on temperature inside the thermokinetic mixer during thermokinetic compounding of a formulation of 20% NIN, 79% HPBCD and 1% magnesium stearate (NIN-HPBCD-MS).

FIG. 65 is a graph reporting example X-ray diffraction (XRD) results for a KPC of 20% NIN, 79% HPBCD and 1% magnesium stearate (NIN-HPBCD-MS).

FIG. 66 is a graph reporting example data from modulated differential scanning calorimetry (mDSC) analysis of a KPC of 20% NIN, 79% HPBCD and 1% magnesium stearate (NIN-HPBCD-MS).

FIG. 67 is a graph reporting example data from in vitro dissolution analysis of aphysical mixture (PM, indicated as “DST-2521.015.3”) of 20% NIN, 79% HPBCD and 1% magnesium stearate (NIN-HPBCD-MS), and a KPC thereof (indicated as “DST-2521.015.2”), using a MicroFLUX™ (Pion Inc., Billerica, Mass., USA) apparatus. Concentration of NIN within the donor compartment of the MicroFLUX™ apparatus is shown.

FIG. 68 is a graph reporting example data from in vitro dissolution analysis of aphysical mixture (PM, indicated as “DST-2521.015.3”) of 20% NIN, 79% HPBCD and 1% magnesium stearate (NIN-HPBCD-MS), and a KPC thereof (indicated as “DST-2521.015.2”), using a MicroFLUX™ (Pion Inc., Billerica, Mass., USA) apparatus. Concentration of NIN within the receiver compartment of the MicroFLUX™ apparatus is shown.

FIG. 69 is a graph reporting example results of 2D NOESY analysis of a 1:1 Nintedanib/HPBCD complex.

FIG. 70 is an example molecular diagram of NIN showing protons labeled B and D.

FIG. 71 is an example molecular diagram of HPBCD showing numbered protons.

### DETAILED DESCRIPTION

The present disclosure relates to pharmaceutical formulations including an inclusion complex of an API with a cyclic oligomer, and methods of forming such pharmaceutical formulations. The inclusion complexes in the pharmaceutical formulations of the present disclosure differ from amorphous solid dispersions in which the API is dispersed between excipient molecules. The inclusion complexes in the pharmaceutical formulations of the present disclosure include an amorphous API that is included within the structure of the cyclic oligomer excipient molecule. In particular, in some embodiments, the inclusion complexes in the pharmaceutical formulations of the present disclosure may be formed using a process that includes thermokinetic compounding of an API and a cyclic oligomer. As described herein, in some embodiments the properties of the inclusion complex of the API and the cyclic oligomer is dependent upon the molar ratio of the API to the cyclic oligomer, and the properties of the API and the cyclic oligomer. The inclusion complexes in pharmaceutical formulations of the present disclosure may provide increased solubility and/or bioavailability of the API, and may correspondingly improve therapeutic properties for administration to patients to treat conditions responsive to the API. In addition, formation of inclusion complexes for a range of different APIs, for example using a process of thermokinetic compounding with high shear mixing as described herein, without the use of solvents or external heat, in solid state, is surprising and unexpected.

### A. PHARMACEUTICAL FORMULATION

A pharmaceutical formulation of the present disclosure includes at least one active pharmaceutical ingredient (API). Unless otherwise specified herein, the API may include the active form of the API and its modified forms, in either amorphous or crystalline states. Examples of modified forms of the API include without limitation a pharmaceutically acceptable salt, ester, derivative, analog, prodrug, hydrate, or solvate thereof.

It is expected that inclusion complexes may contain cyclic oligomers and various APIs and/or various additional APIs, according to the present disclosure.

In general, without limitation to theory, an inclusion complex of the present disclosure may include an API and/or an additional API, wherein the API and/or the additional API is small enough to be able to become physically included inside the cavity of the cyclic oligomer. Typically, a suitable API and/or additional API will at least have a minor axis diameter smaller than the central cavity diameter of the cyclic oligomer.

The term “minor axis” as used herein typically refers to a width and/or depth of the three-dimensional molecular structure of the API and/or the additional API. The molecular structure of some APIs and/or additional APIs may also have a “major axis”, which may refer to the longest dimension of a molecule or may be designated the length of the molecule. Accordingly, a suitable API and/or additional API may have a diameter of a width, depth, or combination of width and depth that is small enough to allow at least a portion of the molecule of the API and/or the additional API (e.g., at least 10%, 20%, 30%, 40%, 50%, 60%, 70%, 80%, 90% or 99.9% of a molecule of the API and/or the additional API) to be able to become physically included, in whole or in part, inside the cavity of one or more molecules of the cyclic oligomer.

The term “minor axis diameter” as used herein may refer to a kinetic diameter of the minor axis of the API and/or additional API.

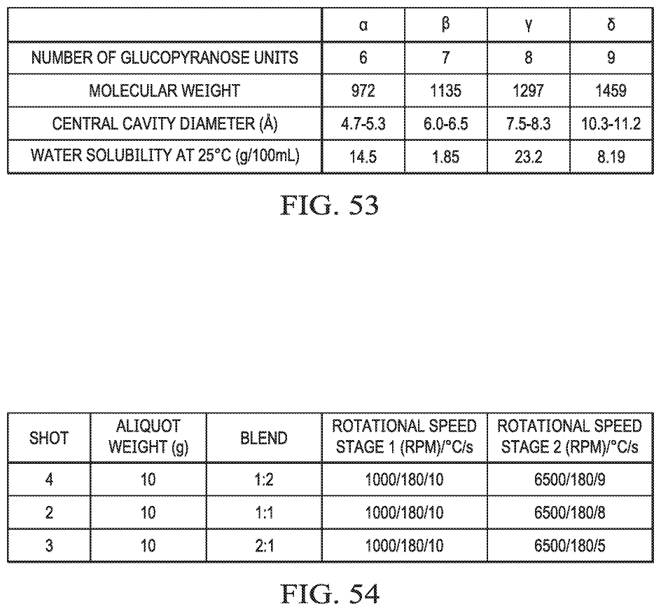

For example, FIG. 53 is a Table of example parameters, including central cavity diameter, in Angstroms (Å), of example cyclic oligomers, including α-cyclodextrin, β-cyclodextrin, γ-cyclodextrin and 6-cyclodextrin. For example, in some embodiments, an API and/or an additional API, having a minor axis diameter up to 4.7-5.3 Å may be sized to be included within the central cavity of α-cyclodextrin. For example, in some embodiments, an API and/or an additional API, having a minor axis diameter up to 6.0-6.5 Å may be sized to be included within the central cavity of β-cyclodextrin. For example, in some embodiments, an API and/or and additional API, having a minor axis diameter up to 7.5-8.3 Å may be sized to be included within the central cavity of γ-cyclodextrin. For example, in some embodiments, an API and/or an additional API, having a minor axis diameter up to 10.3-11.2 Å may be sized to be included within the central cavity of 6-cyclodextrin.

In some embodiments, an API and/or an additional API may be hydrophilic. In some embodiments, an API and/or an additional API may be hydrophobic. In some embodiments, the API and/or the additional API may have a solubility in water of greater than 1 mg/mL. In some embodiments, an API and/or an additional API having a solubility below 1 mg/mL may form more stable inclusion complexes with the cyclic oligomers of the disclosure, including increased physical stability, chemical stability, or both, as described herein.

In some embodiments, an API may be a small molecule drug for treating a patient. The term “small molecule” as used herein generally refers to a low molecular weight (<900 daltons) organic compound capable of regulating a biological process, with a size on the order of 1 nm.

In some embodiments, a patient may be an animal, such as a mammal, such as a human.

For example, in some embodiments, the API may be for treating a disorder of the digestive system, such as antacids, reflux suppressants, antiflatulents, antidopaminergics, proton pump inhibitors (PPIs), H2-receptor antagonists, cytoprotectants, prostaglandin analogues, laxatives, antispasmodics, antidiarrhoeals, bile acid sequestrants, and opioids.

For example, in some embodiments, the API may be for treating a disorder of the cardiovascular system, such as β-receptor blockers, calcium channel blockers, diuretics, cardiac glycosides, antiarrhythmics, nitrate, antianginals, vasoconstrictors, vasodilators, ACE inhibitors, angiotensin receptor blockers, a blockers, calcium channel blockers, thiazide diuretics, loop diuretics, aldosterone inhibitors, anticoagulants, heparin, antiplatelet drugs, fibrinolytics, anti-hemophilic factors, haemostatic drugs, and hypolipidaemic agents.

For example, in some embodiments, the API may be for treating a disorder of the central nervous system, such as psychedelics, hypnotics, anaesthetics, antipsychotics, eugeroics, antidepressants (including tricyclic antidepressants, monoamine oxidase inhibitors, lithium salts, and selective serotonin reuptake inhibitors (SSRIs)), antiemetics, Anticonvulsants/antiepileptics, anxiolytics, barbiturates, movement disorder (e.g., Parkinson's disease) drugs, stimulants (including amphetamines), benzodiazepines, cyclopyrrolones, dopamine antagonists, antihistamines, cholinergics, anticholinergics, emetics, cannabinoids, and 5-HT (serotonin) antagonists.

For example, in some embodiments, the API may be for treating pain, such as analgesics, NSAIDs, opioids and anesthetics, benzodiazepines and barbiturates.

For example, in some embodiments, the API may be for treating a musculoskeletal disorder, for example NSAIDs (including COX-2 selective inhibitors), muscle relaxants, neuromuscular drugs, and anticholinesterases.

For example, in some embodiments, the API may be for treating a disorder of the eye, such as an adrenergic neuron blocker, astringents, topical anesthetics, sympathomimetics, parasympatholytics, mydriatics, cycloplegics, antibiotics, topical antibiotics, sulfa drugs, aminoglycosides, fluoroquinolones, antiviral drugs, imidazoles, polyenes, NSAIDs, corticosteroids, mast cell inhibitors, adrenergic agonists, beta-blockers, carbonic anhydrase inhibitors/hyperosmotics, cholinergics, miotics, parasympathomimetics, prostaglandin agonists/prostaglandin inhibitors, and nitroglycerin.

For example, in some embodiments, the API may be for treating a disorder of the ear, nose or oropharynx, such as antibiotics, sympathomimetics, antihistamines, anticholinergics, NSAIDs, corticosteroids, antiseptics, local anesthetics, antifungals, and cerumenolytics.

For example, in some embodiments, the API may be for treating a disorder of the respiratory system, such as bronchodilators, antitussives, mucolytics, decongestants, corticosteroids, Beta2-adrenergic agonists, anticholinergics, Mast cell stabilizers, and Leukotriene antagonists.

For example, in some embodiments, the API may be for treating a disorder of the endocrine system, such as androgens, antiandrogens, estrogens, gonadotropin, corticosteroids, human growth hormone, insulin, antidiabetics (sulfonylureas, biguanides/metformin, thiazolidinediones, insulin), thyroid hormones, antithyroid drugs, calcitonin, diphosponate, and vasopressin analogues.

For example, in some embodiments, the API may be for treating a disorder of the reproductive system or urinary system, such as antifungals, alkalinizing agents, quinolones, antibiotics, cholinergics, anticholinergics, antispasmodics, 5-alpha reductase inhibitor, selective alpha-1 blockers, sildenafils, and fertility medications.

For example, in some embodiments, the API may be a contraceptive drug, such as a hormonal contraceptive agent, ormeloxifene, or a spermicide.

For example, in some embodiments, the API may be an obstetrics and gynecology drug, such as NSAIDs, anticholinergics, haemostatic drugs, antifibrinolytics, Hormone Replacement Therapy (HRT), bone regulators, beta-receptor agonists, follicle stimulating hormone, luteinising hormone, LHRH gamolenic acid, gonadotropin release inhibitor, progestogen, dopamine agonists, oestrogen, prostaglandins, gonadorelin, clomiphene, tamoxifen, and diethylstilbestrol.

For example, in some embodiments, the API may be for treating a disorder of the skin, such as emollients, anti-pruritics, antifungals, disinfectants, scabicides, pediculicides, tar products, vitamin A derivatives, vitamin D analogues, keratolytics, abrasives, systemic antibiotics, topical antibiotics, hormones, desloughing agents, exudate absorbents, fibrinolytics, proteolytics, sunscreens, antiperspirants, corticosteroids, and immune modulators.

For example, in some embodiments, the API may be for treating infections and infestations, such as antibiotics, antifungals, antileprotics, antituberculous drugs, antimalarials, anthelmintics, amoebicides, antivirals, antiprotozoals, probiotics, prebiotics, antitoxins and antivenoms.

For example, in some embodiments, the API may be for treating a disorder of the immune system, such as an immunosuppressant or an immune stimulant.

For example, in some embodiments, the API may be for treating an allergic disorder, such as anti-allergics, antihistamines, NSAIDs, and corticosteroids.

For example, in some embodiments, the API may be a nutrition drug, such as vitamins, anti-obesity drugs, anabolic drugs, and haematopoietic drugs.

For example, in some embodiments, the API may be for treating a neoplastic disorder, such as cytotoxic drugs, sex hormones, aromatase inhibitors, somatostatin inhibitors, recombinant interleukins, G-CSF, and erythropoietin.

For example, in some embodiments, the API included in the pharmaceutical formulations of the present disclosure may be selected from aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, itraconazole, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, rivaroxaban, RRR-dihydrotetrabenazine (RRR-DHTBZ), SAGE-689, sonidegib, sorafenib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, ziprasidone, and any combinations thereof, including without limitation any other APIs.

The present disclosure describes preparation and analysis of several representative example embodiments of inclusion complexes of APIs with cyclic oligomers (see Examples).

For example, in some representative embodiments, the API may be itraconazole (ITZ, see Example 2). In some embodiments, itraconazole may include the active form of itraconazole and its modified forms, in either amorphous or crystalline states. Modified forms of itraconazole may include a pharmaceutically acceptable salt, ester, derivative, analog, prodrug, hydrate, or solvate thereof.

Itraconazole is a synthetic triazole antifungal agent. Itraconazole in some embodiments is a 1:1:1:1 racemic mixture of four diastereomers (two enantiomeric pairs), each possessing three chiral centers. Itraconazole may be represented by the following structural formula (I):

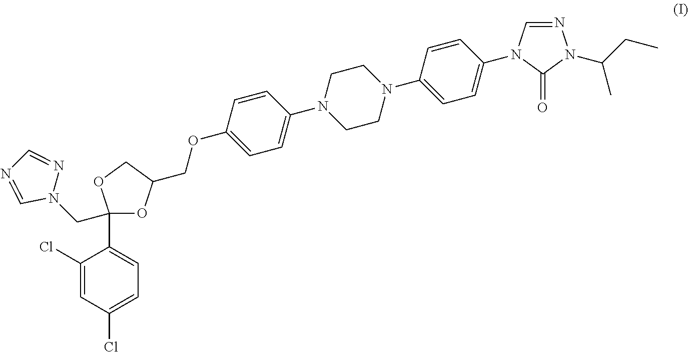 [SMILES: CCC(C)n1ncn(-c2ccc(N3CCN(c4ccc(OCC5COC(Cn6cncn6)(c6ccc(Cl)cc6Cl)O5)cc4)CC3)cc2)c1=O]

and nomenclature: 2-butan-2-yl-4-[4-[4-[4-[[2-(2,4-dichlorophenyl)-2-(1,2,4-triazol-1-ylmethyl)-1,3-dioxolan-4-yl]methoxy]phenyl]-1-piperazinyl]phenyl]-1,2,4-triazol-3-one. Itraconazole has no hydrogen bond donors, 9 hydrogen bond acceptors, and 11 rotatable bonds.

Itraconazole has a molecular formula of C₃₅H₃₈C₁₂N₈O₄ and a molecular weight of 705.64. Itraconazole may be a white to slightly yellowish powder. It is insoluble in water, very slightly soluble in alcohols, and freely soluble in dichloromethane. It has a pKa of 3.70 (based on extrapolation of values obtained from methanolic solutions) and a log (n-octanol/water) partition coefficient of 5.66 at pH 8.1.

In vitro studies have demonstrated that itraconazole inhibits the cytochrome P450-dependent synthesis of ergosterol, which is a vital component of fungal cell membranes. Itraconazole exhibits in vitro activity against Blastomyces dermatitidis, Histoplasma capsulatum, Histoplasma duboisii, Aspergillus flavus, Aspergillus fumigatus, Candida albicans, and Cryptococcus neoformans. Itraconazole also exhibits varying in vitro activity against Sporothrix schenckii, Trichophyton species, Candida krusei, and other Candida species.

Itraconazole is commercially available, for example, in oral formulations such as SPORANOX® (Johnson & Johnson, New Jersey), and ONMEL® (Stiefel Laboratories, Inc., Delaware).

For example, SPORANOX® Capsules are indicated for the treatment of the following fungal infections in immunocompromised and non-immunocompromised patients: 1.

Blastomycosis, pulmonary and extrapulmonary; 2. Histoplasmosis, including chronic cavitary pulmonary disease and disseminated, nonmeningeal histoplasmosis; and 3. Aspergillosis, pulmonary and extrapulmonary, in patients who are intolerant of or who are refractory to amphotericin B therapy. SPORANOX® Capsules are also indicated for the treatment of the following fungal infections in non-immunocompromised patients: 1. Onychomycosis of the toenail, with or without fingernail involvement, due to dermatophytes (Tinea unguium); and 2.

Onychomycosis of the fingernail due to dermatophytes (Tinea unguium).

In another representative example, in some embodiments, the API may be sorafenib (SOR, see Example 3). In some embodiments, sorafenib (SOR) may include the active form of sorafenib and its modified forms, in either amorphous or crystalline states. Modified forms of sorafenib may include a pharmaceutically acceptable salt, ester, derivative, analog, prodrug, hydrate, or solvate thereof.

Sorafenib is a kinase inhibitor that decreases tumor cell proliferation.

For example, Sorafenib in some embodiments may be used to inhibit multiple intracellular (c-CRAF, BRAF and mutant BRAF) and cell surface kinases (KIT, FLT-3, RET, RET/PTC, VEGFR-1, VEGFR-2, VEGFR-3, and PDGFR-ß). Several of these kinases are thought to be involved in tumor cell signaling, angiogenesis and apoptosis. Sorafenib inhibits tumor growth of Hepatocellular Carcinoma (HCC), Renal Cell Carcinoma (RCC), and Differentiated Thyroid Carcinoma (DTC) human tumor xenografts in immunocompromised mice. Reductions in tumor angiogenesis are seen in models of HCC and RCC upon sorafenib treatment and increases in tumor apoptosis were observed in models of HCC, RCC, and DTC.

Sorafenib may be represented by the following structural formula (II):

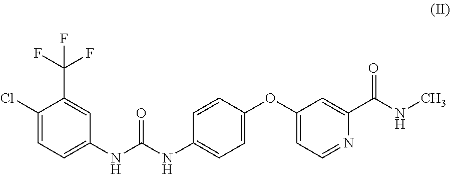 [SMILES: CNC(=O)c1cc(Oc2ccc(NC(=O)Nc3ccc(Cl)c(C(F)(F)F)c3)cc2)ccn1]

and nomenclature: 4-[4-({[4-chloro-3-(trifluoromethyl)phenyl]carbamoyl}amino)phenoxy]-N-methylpyridine-2-carboxamide. Sorafenib has 3 hydrogen bond donors, 7 hydrogen bond acceptors, and 5 rotatable bonds.

Sorafenib has a molecular formula of C₂₁H₁₆ClF₃N₄O₃ and a molecular weight of 464.8 g/mol. Sorafenib may be a white to yellowish or brownish solid. Sorafenib has a melting point of 205.6° C. Sorafenib is practically insoluble in aqueous media, slightly soluble in ethanol and soluble in PEG 400.

Sorafenib is commercially available, for example, in various formulations such as NEXAVAR® (Bayer Aktiengesellschaft, Germany). NEXAVAR® is indicated for the treatment of patients with unresectable hepatocellular carcinoma (HCC), advanced renal cell carcinoma (RCC), or locally recurrent or metastatic, progressive, differentiated thyroid carcinoma (DTC) that is refractory to radioactive iodine treatment (NEXAVAR® Prescribing Information).

In yet another representative example, in some embodiments, the API may be Rivaroxaban (RIV, see Example 4). In some embodiments, Rivaroxaban (RIV) may include the active form of Rivaroxaban and its modified forms, in either amorphous or crystalline states.

Modified forms of Rivaroxaban may include a pharmaceutically acceptable salt, ester, derivative, analog, prodrug, hydrate, or solvate thereof.

Rivaroxaban is a factor Xa inhibitor and an anticoagulant used to prevent venous thromboembolism, myocardial infarction, and stent thrombosis. Activation of factor X to factor Xa (FXa) via the intrinsic and extrinsic pathways plays a central role in the cascade of blood coagulation.

Rivaroxaban may be represented by the following structural formula (III):

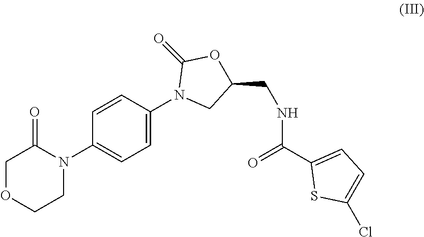 [SMILES: O=C(NC[C@H]1CN(c2ccc(N3CCOCC3=O)cc2)C(=O)O1)c1ccc(Cl)s1]

and nomenclature: 5-Chloro-N-({(5S)-2-oxo-3-[4-(3-oxo-4-morpholinyl)phenyl]-1,3-oxazolidin-5-yl}methyl)-2-thiophenecarboxamide. Rivaroxaban is a pure (S)-enantiomer. Rivaroxaban has one hydrogen bond donor, and 6 hydrogen bond acceptors, and 5 rotatable bonds.

Rivaroxaban has a molecular formula of C₁₉H₁₈ClN₃O₅S and a molecular weight of 435.89 g/mol. Rivaroxaban may be an odorless, non-hygroscopic, white to yellowish powder. Rivaroxaban has a melting point of 230° C. Rivaroxaban is only slightly soluble in organic solvents (e.g., acetone, polyethylene glycol 400) and is practically insoluble in water and aqueous media.

Rivaroxaban is commercially available, for example in various formulations such as XARELTO® (Bayer Aktiengesellschaft, Germany). XARELTO® is indicated to reduce the risk of stroke and systemic embolism in patients with nonvalvular atrial fibrillation, or for the prophylaxis of deep vein thrombosis (DVT), which may lead to pulmonary embolism (PE) in patients undergoing knee or hip replacement surgery (XARELTO® Prescribing Information).

In another representative example, in some embodiments, the API may be Nintedanib (NIN, see Example 5). In some embodiments, Nintedanib may include the active form of Nintedanib and its modified forms, in either amorphous or crystalline states. Modified forms of Nintedanib may include a pharmaceutically acceptable salt, ester, derivative, analog, prodrug, hydrate, or solvate thereof.

Nintedanib is used to treat idiopathic pulmonary fibrosis (IPF; scarring of the lungs with an unknown cause). It is also used to treat certain types of chronic fibrosing interstitial lung diseases (ILD; an ongoing disease in which there is increased scarring of the lungs). Nintedanib is also used to slow the rate of decline in lung function in people with systemic sclerosis-associated interstitial lung disease (SSc-ILD; also known as scleroderma-associated ILD: a disease in which there is scarring of the lungs that is often fatal).

Nintedanib is a kinase inhibitor and functions to block the action of enzymes involved in causing fibrosis. Nintedanib inhibits multiple receptor tyrosine kinases (RTKs) and non-receptor tyrosine kinases (nRTKs). Nintedanib inhibits the following RTKs: platelet-derived growth factor receptor (PDGFR) α and β, fibroblast growth factor receptor (FGFR) 1-3, vascular endothelial growth factor receptor (VEGFR) 1-3, colony stimulating factor 1 receptor (CSF1R), and Fms-like tyrosine kinase-3 (FLT-3). These kinases except for FLT-3 have been implicated in pathogenesis of interstitial lung diseases (ILD). Nintedanib binds competitively to the adenosine triphosphate (ATP) binding pocket of these kinases and blocks the intracellular signaling cascades, which have been demonstrated to be involved in the pathogenesis of fibrotic tissue remodeling in ILD. Nintedanib also inhibits the following nRTKs: Lck, Lyn and Src kinases.

Nintedanib may be represented by the following structural formula (IV):

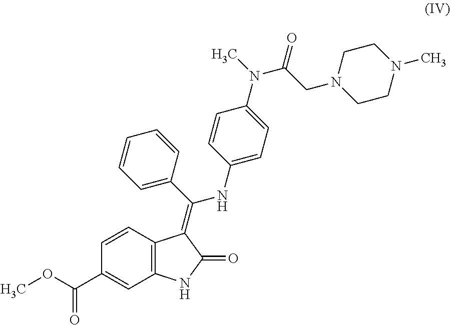

and nomenclature: 1H-Indole-6-carboxylic acid, 2,3 dihydro-3-[[[4-[methyl[(4-methyl-1-piperazinyl)acetyl]amino]phenyl]amino]phenylmethylene]-2-oxo-,methyl ester. In some embodiments, Nintedanib may be present as ethanesulfonate salt (esylate), with the chemical name 1H-Indole-6-carboxylic acid, 2,3 dihydro-3-[[[4-[methyl[(4-methyl-1-piperazinyl)acetyl]amino]phenyl]amino]phenylmethylene]-2-oxo-,methyl ester, (3Z)-, ethanesulfonate (1:1).

For example, Nintedanib esylate is a bright yellow powder with an empirical formula of C₃₁H₃₃N₅O₄.C₂H₆O₃S and a molecular weight of 649.76 g/mol.

Nintedanib displays a pH-dependent solubility profile with increased solubility at acidic pH less than 3. For example, Nintedanib esylate is soluble in water. A saturated solution in water was found to have a concentration of 2.8 mg/mL and exhibited an intrinsic pH of 5.7. The solubility of nintedanib esilate is strongly pH dependent with an increased solubility at acidic pH, particularly for pH<3.

Nintedanib is commercially available, for example in various formulations such as OFEV® (Boehringer Ingelheim International GmbH, Germany). OFEV® is indicated for treatment of idiopathic pulmonary fibrosis (IPF) and for slowing the rate of decline in pulmonary function in patients with systemic sclerosis-associated interstitial lung disease (OFEV® Prescribing Information).

In certain embodiments, the API excludes abiraterone, including both the active form of abiraterone and its modified forms, in either amorphous or crystalline states. Modified forms of abiraterone include a pharmaceutically acceptable salt, ester, derivative, analog, prodrug, hydrate, or solvate thereof, in particular abiraterone acetate.

The API in a pharmaceutical formulation of the present disclosure may lack substantial impurities. For example, the API may lack impurities at levels beyond the threshold that has been qualified by toxicology studies for the API, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the API in a pharmaceutical formulation of the present disclosure may have less than 1.0%, 0.75%, 0.5%, 0.1%, 0.05%, or 0.01% impurities by weight as compared to total weight of the API and impurities, relative to a standard of known concentration in mg/mL. As another alternative, the API in a pharmaceutical formulation of the present disclosure may retain at least 90%, 91%, 92%, 93%, 94%, 95%, 96%, 97%, 98%, 99%, or 99.9% drug activity or potency as compared to the uncompounded API as measured by HPLC. Impurities may include API degradation products, such as thermal degradation products.

A pharmaceutical formulation of the present disclosure may further include one or more additional APIs in addition to a first API. In some embodiments, suitable additional APIs include other APIs approved to treat a condition or disorder responsive to treatment by the first API, or a side effect of the condition or disorder, or a side-effect of the first API. In some embodiments, suitable additional APIs include other APIs approved to treat a different condition or disorder than the condition or disorder responsive to treatment by the first API, or a side effect of the different condition or disorder. These additional APIs may be in their active form or modified forms. These additional APIs may be compoundable even when they have not been previously compoundable, compoundable in an administrable pharmaceutical formulation such as an orally administrable or parenterally administrable pharmaceutical formulation, compoundable together with another API, or compoundable in their active forms. Additional APIs may be in the same inclusion complex as the first API, in a second inclusion complex that does not include the first API, or otherwise in the pharmaceutical formulation, but not in an inclusion complex.

Any additional API in a pharmaceutical formulation of the present disclosure may also not contain substantial levels of impurities. For example, the additional API may not have impurities at levels beyond the threshold that has been qualified by toxicology studies, or beyond the allowable threshold for unknown impurities as established in the Guidance for Industry, Q3B(R2) Impurities in New Drug Products (International Committee for Harmonization, published by the U.S. Department of Health and Human Services, Food and Drug Administration, Center for Drug Evaluation and Research (CDER), Center for Biologics Evaluation and Research, July, 2006, incorporated by reference herein. Alternatively, the additional API in a pharmaceutical formulation of the present disclosure may be have less than 1.0%, 0.75%, 0.5%, 0.1%, 0.05%, or 0.01% impurities by weight as compared to total weight of additional API and impurities, relative to a standard of known concentration in mg/mL. As another alternative, the additional API in a pharmaceutical formulation of the present disclosure may retain at least 90%, 91%, 92%, 93%, 94%, 95%, 96%, 97%, 98%, 99%, or 99.9% drug activity or potency as compared to the uncompounded additional API as measured by HPLC. Impurities may include additional API degradation products, such as thermal degradation products.

A pharmaceutical formulation of the present disclosure further includes at least one excipient. When multiple excipients are used in a pharmaceutical formulation of the present disclosure, the one present in the largest amount by weight percent is typically referred to as the primary excipient, with other excipients being designated the secondary excipient, tertiary excipient and so forth based on descending amounts by weight percent in the pharmaceutical formulation.

A pharmaceutical formulation of the present disclosure includes a cyclic oligomer excipient. At least one cyclic oligomer excipient is in an inclusion complex with the API in a pharmaceutical formulation of the present disclosure. In some embodiments, an inclusion complex may include two or more cyclic oligomers. In other embodiments, other cyclic oligomer excipients may be present, but not in an inclusion complex. A cyclic oligomer excipient may include a cyclic oligosaccharide or cyclic oligosaccharide derivative excipient, a cyclic peptide oligomer or cyclic peptide oligomer derivative, or a cyclic polycarbonate oligomer or cyclic polycarbonate oligomer derivative, and any combinations thereof. An oligosaccharide excipient may have between 3 to 15 saccharide monomer units, such as glucose units and glucose derivative units, fructose units and fructose derivative units, galactose and galactose derivative units, and any combinations thereof. The saccharide monomer units may be derivatized with a functional group, for example a sulfobutylether, or a hydroxypropyl derivative, or a carboxymethyl derivative or by methylation.

For example, the pharmaceutical formulation of the present disclosure may include a cyclodextrin (CD).

Cyclodextrins (CDs) are cyclic oligomers containing at least six D-(+)-glucopyranose units attached by α(1→4) glycosidic bonds (Davis, Mark E., and Marcus E. Brewster. 2004. ‘Cyclodextrin-based pharmaceutics: past, present and future’, Nature Reviews Drug Discovery, 3: 1023-35; Loftsson, T., P. Jarho, M. Masson, and T. Jarvinen. 2005. ‘Cyclodextrins in drug delivery’, Expert Opin Drug Deliv, 2: 335-51). The glucopyranose units in CDs are present in chair conformation, thereby giving CDs a truncated cone like or toroidal structure (Loftsson, T., P. Jarho, M. Masson, and T. Jarvinen. 2005. ‘Cyclodextrins in drug delivery’, Expert Opin Drug Deliv, 2: 335-51). The outer surface of CDs have secondary hydroxy groups extending from the wider edge and the primary groups from the narrow edge of the cone, which imparts hydrophilic nature to the outer surface (Sharma, Neha, and Ashish Baldi. 2016. ‘Exploring versatile applications of cyclodextrins: an overview’, Drug Delivery, 23: 729-47). The inner cavity of CDs contain skeletal carbons with hydrogen atoms and oxygen bridges, which imparts lipophilic nature to the inner cavity (Sharma, Neha, and Ashish Baldi. 2016. ‘Exploring versatile applications of cyclodextrins: an overview’, Drug Delivery, 23: 729-47).

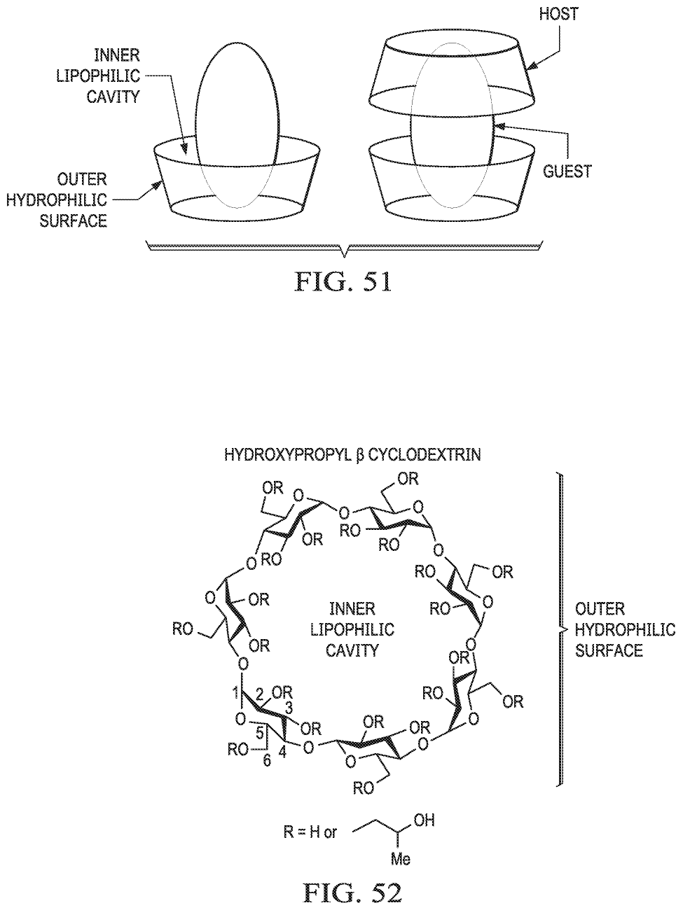

As shown for example in schematic form in FIG. 51, CDs can form inclusion complexes with an entire API or a portion of the API, by including the API into its lipophilic central cavity through non-covalent interactions (Saokham, P., C. Muankaew, P. Jansook, and T. Loftsson. 2018. ‘Solubility of Cyclodextrins and Drug/Cyclodextrin Complexes’, Molecules, 23). By forming partial or complete inclusion complexes with APIs, CDs can impart higher aqueous solubility to an API and increase bioavailability of the API (Saokham, P., C. Muankaew, P. Jansook, and T. Loftsson. 2018. ‘Solubility of Cyclodextrins and Drug/Cyclodextrin Complexes’, Molecules, 23). Accordingly, CDs may be used as excipients of poorly water-soluble APIs (e.g., see Varma, M. M., and P. S. Kumar. 2012. ‘Formulation and Evaluation of GLZ Tablets Containing PVP K30 and Hydroxyl Propyl Beta Cyclodextrin Solid Dispersion’, International Journal of Pharmaceutical Sciences and Nanotechnology, 5: 1706-19; Yuvaraja, K., and Jasmina Khanam. 2014. ‘Enhancement of carvedilol solubility by solid dispersion technique using cyclodextrins, water soluble polymers and hydroxyl acid’, Journal of Pharmaceutical and Biomedical Analysis, 96: 10-20).

The cyclodextrins (CDs) of the present disclosure include, without limitation, α-CD, β-CD, γ-CD, and δ-CD containing six, seven, eight and nine glucopyranose units respectively (Loftsson, T., P. Jarho, M. Masson, and T. Jarvinen. 2005. ‘Cyclodextrins in drug delivery’, Expert Opin Drug Deliv, 2: 335-51), and derivatives thereof.

The CDs of the present disclosure may include substitutions of one or more hydroxy groups. Without limitation to theory, substitution of hydroxy groups of CDs with hydrophobic or hydrophilic groups may be used to impart higher aqueous solubility to the CDs by interrupting their intermolecular hydrogen bonding. For example, hydroxypropyl substitution on R-CD to form hydroxy propyl β cyclodextrin (HPBCD) (FIG. 52) increases its water solubility from 18.5 mg/ml to >600 mg/ml respectively (Loftsson, T., P. Jarho, M. Masson, and T. Jarvinen. 2005. ‘Cyclodextrins in drug delivery’, Expert Opin Drug Deliv, 2: 335-51).

Accordingly, in some embodiments, the R-CD may include one or more R-groups for example as shown in FIG. 52, wherein R=CH₂CHOHCH₃ or H, having varying degrees of substitution at the 2, 3, and 6 positions).

In some embodiments, the cyclodextrin may be a cyclodextrin containing 6, 7, 8 or 9 monomer units, in particular an α cyclodextrin, such as CAVAMAX® W6 Pharma (Wacker Chemie AG, Germany), a β cyclodextrin, such as CAVAMAX® W7 Pharma (Wacker Chemie), or a γcyclodextrin, such as CAVAMAX® W8 Pharma (Wacker Chemie). Suitable cyclodextrins also include hydroxypropyl β cyclodextrin (HPBCD), such as KLEPTOSE® HBP (Roquette, France) and Na sulfo-butyl ether β cyclodextrin, such as DEXOLVE® 7 (Cyclolab, Ltd., Hungary).

Derivatization may facilitate the use of cyclic oligomer excipients in thermokinetic compounding. Particularly when used in a thermokinetic compounding process, particle size of a cyclic oligomer excipient may facilitate compounding. Derivatization, pre-treatment such as by slugging and/or granulation, may increase or decrease particle size of a cyclic oligomer excipient to be within an optimal range. For example, the average particle size of a cyclic oligomer excipient may be increased by up to 500%, or up to 1,000%, by between 50% and 500%, or by between 50% and 1,000%. The average particle size of a cyclic oligomer excipient may be decreased by up to 50%, or up to 90%, or by between 5% and 50% or by between 5% and 90%.

The inclusion complexes of the present disclosure may be referred to as a host-guest inclusion complex, in which a cyclic oligomer is the host and the API is the guest. For example, FIG. 51 shows in schematic form, non-limiting examples of host-guest inclusion complexes. In one non-limiting example, FIG. 51, left Panel, schematically depicts one unit of a host molecule, such as a cyclic oligomer, e.g., a cyclodextrin such as hydroxypropyl β cyclodextrin (HPBCD), in which a guest molecule, the API is included, or at least a portion of the API molecule is included. In another non-limiting example, FIG. 51, right Panel, schematically depicts two units of a host molecule, such as a cyclic oligomer, e.g., a cyclodextrin such as hydroxypropyl β cyclodextrin (HPBCD), in which a guest molecule, the API, is included, or at least a portion of the API molecule is included.

Accordingly, in some embodiments, the present disclosure provides a pharmaceutical formulation comprising (i) an API or a pharmaceutically acceptable salt, ester, derivative, analog, prodrug, hydrate, or solvate thereof, or any combination thereof, wherein the API is not abiraterone or a pharmaceutically acceptable salt, ester, derivative, analog, prodrug, hydrate, or solvate thereof; and (ii) a cyclic oligomer; wherein at least a portion of the API is present in an inclusion complex with the cyclic oligomer.

A pharmaceutical formulation of the present disclosure may be prepared by thermokinetically processing the API and the cyclic oligomer, as described herein.

Accordingly, in some embodiments, a pharmaceutical formulation of the present disclosure may include an inclusion complex, wherein at least a portion of the API is present in the inclusion complex with the cyclic oligomer, and wherein the pharmaceutical formulation is formed by a method comprising thermokinetically processing the API and the cyclic oligomer, and wherein the API may be a pharmaceutically acceptable salt, ester, derivative, analog, prodrug, hydrate, or solvate thereof, or any combination thereof, wherein the API is not abiraterone or a pharmaceutically acceptable salt, ester, derivative, analog, prodrug, hydrate, or solvate thereof.

In some embodiments, the thermokinetic processing may be performed at an average temperature at or below the melting point of one or more of the APIs or excipients. In some embodiments, the thermokinetic processing may be performed at an average temperature at or below the glass transition temperature of one or more of the APIs or excipients. In some embodiments, the thermokinetic processing may be performed at an average temperature at or below the molten transition point of one or more of the APIs or excipients.

For example, and without limitation, in some embodiments, a pharmaceutical formulation of the present disclosure may be thermokinetically processed at a temperature less than or equal to 300° C., such as at a temperature of less than 290° C., 280° C., 270° C., 260° C., 250° C., 240° C., 230° C., 220° C., 210° C., 200° C., 190° C., 180° C., 170° C., 160° C., 150° C., 140° C., 130° C., 120° C., 110° C., 100° C., 90° C., 80° C., 70° C., 60° C., 50° C., 40° C., 30° C., 20° C., or 10° C.

In some embodiments, a pharmaceutical formulation of the present disclosure may be thermokinetically processed for less than 300 seconds to form an inclusion complex of the API and the cyclic oligomer. For example, in some embodiments, a pharmaceutical formulation of the present disclosure may be thermokinetically processed for less than 290 seconds, 280 seconds, 270 seconds, 260 seconds, 250 seconds, 240 seconds, 230 seconds, 220 seconds, 210 seconds, 200 seconds, 190 seconds, 180 seconds, 170 seconds, 160 seconds, 150 seconds, 140 seconds, 130 seconds, 120 seconds, 110 seconds, 100 seconds, 90 seconds, 80 seconds, 70 seconds, 60 seconds, 50 seconds, 40 seconds, 30 seconds, 20 seconds, 10 seconds, 9 seconds, 8 seconds, 7 seconds, 6 seconds, 5 seconds, 4 seconds, 3 seconds, or 2 seconds.

Without being limited by theory, thermokinetic compounding of the API together with the cyclic oligomer may provide an advantage in formulating the pharmaceutical formulations of the present disclosure such that the thermokinetic compounding process allows more intimate mixing of the API with the cyclic oligomer than is possible using some other methods of formulation. Without limitation to theory, in some implementations, thermokinetic compounding may allow the API to become included by the truncated cone like or toroidal structure of the cyclodextrin. The thermokinetic compounding may provide increased efficiency and/or percentage of inclusion of the available API within the cyclic oligomer structure, e.g., within the cyclodextrin structure, compared to other formulation methods. Accordingly, the inclusion of the API within the cyclodextrin may provide improved solubility and bioavailability of the API.

In some embodiments, formulating the inclusion complexes of the present disclosure at an industrial scale may be possible using a method that includes a thermokinetic compounding process, when formulating the inclusion complexes of the present disclosure at an industrial scale may not be possible using other methods that do not include a thermokinetic compounding process. In some embodiments, formulating the inclusion complexes of the present disclosure at an industrial scale using a method that includes a thermokinetic compounding process may be more efficient and/or less costly than when formulating the inclusion complexes of the present disclosure at an industrial scale using other methods that do not include a thermokinetic compounding process.

In some embodiments, the present disclosure relates to methods of forming a pharmaceutical formulation of an API and a cyclic oligomer having an optimal drug loading of the API in an inclusion complex with the cyclic oligomer.

The term “drug loading” generally refers to the amount of API incorporated into the pharmaceutical formulation. In particular, the term “drug loading” as used herein refers to the amount of API that can be included in an inclusion complex within one or more cyclic oligomers in the pharmaceutical formulation. In some embodiments, a method of forming a pharmaceutical formulation of the present disclosure that includes a thermokinetic compounding process provides increased drug loading as compared to other methods of formulating pharmaceutical formulations, meaning an increased amount of API that can be included in an inclusion complex within one or more cyclic oligomers in the pharmaceutical formulation, and a corresponding decrease in the amount of unincluded API that may be present as an amorphous dispersion between the cyclic oligomers of the pharmaceutical formulation.

For example, in general for solid dispersions, when the drug loading is less than the equilibrium solubility of the crystalline drug in the polymer carrier, the system is thermodynamically stable and the drug is molecularly dispersed in the polymer carrier matrix, forming a homogenous system (Huang and Dai 2014). Such a system may not be practical since for some drug-polymer carrier systems, as this would occur at extremely low drug loadings (Huang and Dai 2014). When the drug loading is high in solid dispersions, such as when it is higher than the equilibrium solubility of the amorphous drug in a polymer carrier, such systems are highly unstable and can lead to spontaneous phase separation and crystallization, thereby negatively affecting the stability and performance of solid dispersions (Qian, Huang, and Hussain 2010). Moreover, when cyclic oligomers such as CDs are excipients for pharmaceutical formulations, it cannot be assumed that low drug loading is necessarily beneficial. This is because low drug loading may mean a higher amount of CD, which may hamper drug absorption from the gastrointestinal tract and lead to lower bioavailability (Loftsson et al. 2016; Loftsson and Brewster 2012). Also, problems associated with high drug loading as discussed above may also be true for pharmaceutical formulations including an API and a cyclic oligomer, for example when an API is present in higher than the equilibrium solubility of the amorphous drug in a cyclic oligomer carrier, such systems may be highly unstable and can lead to spontaneous phase separation and crystallization, thereby negatively affecting the stability and performance of the inclusion complex. Thus, identification of optimal drug loading is important when the carrier is a cyclic oligomer, for example such as HPBCD.

In some embodiments, a pharmaceutical formulation of the present disclosure may have drug loading of up to 10%, 20%, 30%, 40%, 50%, 60%, 70%, 80%, or 90% weight of the API as a total weight of the pharmaceutical formulation including the weight of the API plus the weight of the cyclic oligomer and optionally other excipients. As would be understood by skilled persons, for a given combination of API and cyclic oligomer, the relationship between percentage weight of drug loading and molar ratio of API to cyclic oligomer may be calculated by taking into account the molecular weight (e.g., in g/mol) of each of the API and cyclic oligomer.

In some embodiments, a method of formulation that includes a thermokinetic compounding process provides increased inclusion complexation efficiency and correspondingly increased stability of amorphous API in the pharmaceutical formulation as compared to a pharmaceutical formulation having a same molar ratio of an API:cyclic oligomer formed using a method that does not include a thermokinetic compounding process. Without limitation to theory, thermokinetic compounding may allow increased stable inclusion complexation of an API at least partially within the interior of a cyclic oligomer, whereas other methods of formulation that do not include a thermokinetic compounding process may have an increased amount of unincluded API present outside the cyclic oligomer.

Accordingly, in some embodiments, the present disclosure provides a pharmaceutical formulation including an active pharmaceutical ingredient (API), or a pharmaceutically acceptable salt, ester, derivative, analog, prodrug, hydrate, or solvate thereof; and a cyclic oligomer excipient; wherein at least 90%, 91%, 92%, 93%, 94%, 95%, 96%, 97%, 98%, 99%, or 99.9% of the API in the pharmaceutical formulation is present as an inclusion complex within the cyclic oligomer. In some embodiments, all of the API in the pharmaceutical formulation is present as an inclusion complex within the cyclic oligomer.

In particular, in some embodiments, the present disclosure provides a pharmaceutical formulation including an active pharmaceutical ingredient (API) selected from itraconazole (ITZ), sorafenib (SOR), rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, and ziprasidone, or a pharmaceutically acceptable salt, ester, derivative, analog, prodrug, hydrate, or solvate thereof; and a cyclic oligomer excipient; wherein at least 90%, 91%, 92%, 93%, 94%, 95%, 96%, 97%, 98%, 99%, or 99.9% of the API is present as an inclusion complex within the cyclic oligomer.

In some embodiments of the pharmaceutical formulation described herein, the API may include less than 10%, 9%, 8%, 7%, 6%, 5%, 4%, 3%, 2%, 1%, or 0.1% crystalline API.

In some embodiments described herein, the inclusion complex may have a molar ratio of guest molecule:host molecule from 1:0.25 to 1:25, for example such as, or such as about, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, or 1.3.0.

Accordingly, in some embodiments of the pharmaceutical formulation of the present disclosure, the API and the cyclic oligomer may be present in the pharmaceutical formulation in a molar ratio of API:cyclic oligomer from 1:0.25 to 1:25, for example such as, or such as about, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, or 1.3.0.

For example, in some embodiments described herein, the inclusion complex in the pharmaceutical formulation include hydroxypropyl β cyclodextrin (HPBCD) and an API selected from Itraconazole (ITZ), Sorafenib (SOR), Rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, and ziprasidone, wherein a molar ratio of the HPBCD:API is from 1:0.25 to 1:25, for example such as, or such as about, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1:1.9, 1:2.0, 1:2.1, 1:2.2, 1:2.3, 1:2.4, 1:2.5, 1:2.6, 1:2.7, 1:2.8, 1:2.9, or 1.3.0. For example, see Examples 2 to 5. In some embodiments, the inclusion complex in the pharmaceutical formulation is formed using a process that includes thermokinetic compounding (see Example 2 to 5).

In some embodiments, a pharmaceutical formulation of the present disclosure formed using a method that includes a thermokinetic compounding process may provide an increase of up to 10%, 20%, 30%, 40%, 50%, 60%, 70%, 80%, or 90% more inclusion of an API within a cyclic oligomer as compared to a method that does not include a thermokinetic compounding process, or up to 2-fold, 3-fold, 4-fold, 5-fold, 6-fold, 7-fold, 8-fold, 9-fold, 10-fold, or 20-fold increase in inclusion of an API within a cyclic oligomer as compared to a method that does not include a thermokinetic compounding process.

In some embodiments, the inclusion complexes in the pharmaceutical formulations of the present disclosure may have increased stability, both in terms of physical stability and chemical stability. The term “stability” as used herein includes physical stability, such as the API being maintained in an amorphous state in the pharmaceutical formulation, without crystallization or recrystallization of the API. The term “stability” as used herein also refers to chemical stability, such as reduced incidence of API degradation, for example due to incompatibility with other excipients, heat exposure and light exposure. For example, the inclusion complexes of the present disclosure may have reduced crystallization of the API over time as compared to an amount of crystallization over time in a formulation including an API in an amorphous solid dispersion that does not include the API in an inclusion complex. For example, stability of an API in a pharmaceutical formulation of the present disclosure can be assessed using methods known in the art and identifiable by skilled persons upon reading the present disclosure, including but not limited to methods described in the Examples, such as heating studies described herein, wherein a pharmaceutical formulation is heated at a selected temperature for a selected period of time, allowed to cool, and analyzed by X-ray diffraction (XRD). For instance, in the pharmaceutical formulations of the Examples the ITZ-HPBCD pharmaceutical formulation was heated to 90° C. for 6 hours, which is about 60° C. below the temperature of API melting onset. The example SOR-HPBCD pharmaceutical formulation was heated to 150° C. for 6 hours. The example RIV-HPBCD pharmaceutical formulation was heated to 200° C. for 6 hours, which is about 30° C. below the temperature of API melting onset.

Without being bound to any particular theory, when each molecule of an API is complexed with at least one molecule of a cyclic oligomer, each API may be thermally and kinetically stabilized by inclusion of at least a portion of the API within at least one cyclic oligomer, such as shown schematically in FIG. 51, left Panel. Increasing the ratio of cyclic oligomer to API in some embodiments allows inclusion of the API within one or more, such as two, cyclic oligomers, as shown for example in FIG. 51, right Panel. Recrystallization of API that is unincluded within the cyclic oligomer can be detected as crystalline API by XRD analysis. For example, without being bound to any particular theory, API in a pharmaceutical formulation of an API and a cyclic oligomer that may become recrystallized upon heating and subsequent cooling, and detected as crystalline form of the API using, e.g., XRD analysis, may be API that is not included within the cyclic oligomer, but rather may be present as an amorphous API dispersed between the cyclic oligomers in the pharmaceutical formulation.

Accordingly, in some embodiments of the pharmaceutical formulation of the present disclosure, in response to heating the pharmaceutical formulation to a temperature up to 90% of the melting point of the crystalline form of the API and allowing the pharmaceutical formulation to cool to room temperature, less than 10%, 9%, 8%, 7%, 6%, 5%, 4%, 3%, 2%, 1%, or 0.1% of the API may be in crystalline form.

In some embodiments, a pharmaceutical formulation of the present disclosure formed using a method that includes a thermokinetic compounding process may provide an increase in stability of an amorphous API of up to 10%, 20%, 30%, 40%, 50%, 60%, 70%, 80%, 90% or more, as compared to a method that does not include a thermokinetic compounding process, or up to 2-fold, 3-fold, 4-fold, 5-fold, 6-fold, 7-fold, 8-fold, 9-fold, 10-fold, or 20-fold increase in stability of an amorphous API as compared to a method that does not include a thermokinetic compounding process.

Stability analysis can include analysis of pharmaceutical formulations of the present disclosure formed using one or more molar ratios of API:cyclic oligomer, in order to identify a molar ratio of API:cyclic oligomer that provides the highest drug loading of the API in the pharmaceutical formulation that has an acceptable level of stability.

In some embodiments, an acceptable level of stability may be substantially complete amorphicity, having substantially no re-crystallization of the API after heating, or, for example, less than 10%, 9%, 8%, 7%, 6%, 5%, 4%, 3%, 2%, 1%, or 0.1% re-crystallization of API after heating, as determined by X-ray diffraction.

Other methods that can be used to assess stability of the API in the pharmaceutical formulations of the present disclosure include modulated differential scanning calorimetry (mDSC), and Raman spectroscopy (e.g., see Examples).

In general, the amorphous nature or the extent of inclusion of the API within the cyclic oligomer of the inclusion complex may be analyzed using X-ray diffraction (XRD), which may not exhibit strong peaks characteristic of a largely crystalline material. The amorphous nature or the extent of inclusion of the API within the cyclic oligomer of the inclusion complex may also be analyzed using other methods described herein, such as modulated differential scanning calorimetry (mDSC), solid state Nuclear Magnetic Resonance Spectroscopy (ssNMR), Raman spectroscopy, phase solubility analysis, stability analysis, and in vitro dissolution studies. Examples of these methods are described in Example 1 of the present disclosure. For example, the extent of API-cyclic oligomer inclusion complex formation in the pharmaceutical formulation may be evidenced by decrease in intensity of drug melting endotherm in differential scanning calorimetry, or by the magnitude of peak shifts in Raman spectroscopy, or by peak broadening in nuclear magnetic resonance spectroscopy.

A pharmaceutical formulation of the present disclosure may also include one or more additional excipients, one or more additional additives, or both.

It is contemplated that selection of one or more suitable additional excipients for adding to a pharmaceutical formulation that includes an inclusion complex of a particular API and a cyclic oligomer may require screening and testing of a large number of candidate additional excipients. In particular, it is contemplated that selecting one or more suitable additional excipients that provide one or more possible performance enhancing effects of the pharmaceutical formulation in terms of stability, solubility, bioavailablility, and so on, will not be routine or predictable, but rather certain unpredictable combinations may provide surprising performance enhancing results.

The additional excipients may include a polymer excipient or combination of polymer excipients. For example, in some embodiments, a polymer excipient or combination of polymer excipients may be added externally to an inclusion complex. Suitable polymer excipients may be water-soluble. Suitable polymer excipients may also be ionic or non-ionic.

Suitable polymer excipients include without limitation a cellulose-based polymer, a polyvinyl-based polymer, or an acrylate-based polymer. These polymers may have varying degrees of polymerization or functional groups.

Suitable cellulose-based polymers include an alkylcellulose, such as a methyl cellulose, a hydroxyalkylcellulose, or a hydroxyalkyl alkylcellulose. Suitable cellulose-based polymers more particularly include hydroxymethylcellulose, hydroxyethyl methylcellulose, hydroxyethylcellulose, hydroxypropylcellulose, hydroxybutylcellulose, hydroxypropylcellulose, hydroxypropylmethylcellulose, such as METHOCEL™ E3 and METHOCEL™ E5 (Dow Chemical, Michigan, US); ethylcellulose, such as ETHOCEL® (Dow Chemical), cellulose acetate butyrate, hydroxyethylcellulose, sodium carboxymethyl-cellulose, hydroxypropylmethylcellulose acetate succinate, such as AFFINISOL® HPMCAS 126 G (Dow Chemical), cellulose acetate, cellulose acetate phthalate, such as AQUATERIC™ (FMC, Pennsylvania, US), carboxymethylcellulose, such as sodium carboxymethycellulose, hydroxyethyl methyl cellulose, hydroxypropyl cellulose, hydroxyethyl cellulose, hydroxymethyl cellulose, crystalline cellulose, and any combinations thereof.

Suitable polyvinyl-based polymers include polyvinyl alcohol, such as polyvinyl alcohol 4-88, such as EMPROVE® (Millipore Sigma, Massachusetts, US) polyvinyl pyrrolidone, such as LUVITEK® (BASF, Germany) and KOLLIDON® 30 (BASF), polyvinylpyrrolidone-co-vinylacetate, poly(vinyl acetate)-co-poly(vinylpyrrolidone) copolymer, such as KOLLIDON® SR (BASF), poly(vinyl acetate) phthalate, such as COATERIC® (Berwind Pharmaceutical Services, Pennsylvania, US) or PHTHALAVIN® (Berwind Pharmaceutical Services), polyvinyl caprolactam-polyvinyl acetate-polyethylene glycol graft copolymer, such as SOLUPLUS® (BASF), polyvinyl caprolactam-polyvinyl acetate-polyethylene glycol graft copolymer, such as SOLUPLUS® (BASF), hard polyvinylchloride, and any combinations thereof.

Suitable acrylate-based polymers include acrylate and methacrylate copolymer, type A copolymer of ethylacrylate, methyl methacrylate and a methacrylic acid ester with quaternary ammonium groups in a ratio of 1:2:0.1, such as EUDRAGIT® RS PO (Evonik, Germany), poly(meth)acrylate with a carboxylic acid functional group, such as EUDRAGIT® S100 (Evonik), dimethylaminoethyl methacrylate-methacrylic acid ester copolymer, ethylacrylate-methylmethacrylate copolymer, poly(methacrylate ethylacrylate) (1:1) copolymer, poly(methacrylate methylmethacrylate) (1:1) copolymer, poly(methacrylate methylmethacrylate) (1:2) copolymer, poly(methacrylic acid-co-ethyl acrylate) (1:1), such as EUDRAGIT® L-30-D (Evonik), poly(methacylic acid-co-ethyl acrylate) (1:1), such as EUDRAGIT® L100-55 (Evonik), poly(butyl methacylate-co-(2-dimethylaminoethyl) methacrylate-co-methyl methacrylate (1:2:1), such as EUDRAGIT® EPO (Evonik), methacrylic acid-ethacrylate copolymer, such as KOLLICOAT MAE 100-55 (BASF), polyacrylate, polymethacrylate, and any combinations thereof.

The secondary polymer excipient may be water-soluble. The polymer secondary excipient may be ionic or non-ionic. Suitable secondary non-ionic polymer excipients include hydroxy propyl methyl cellulose, such as METHOCEL™ E15 (Dow Chemical, Michigan, US) or METHOCEL™ E50 (Dow Chemical), and polyvinylpyrrolidone, such as KOLLIDON® 90 (BASF, Germany). Suitable secondary ionic polymer excipients include hydroxy propyl methyl cellulose acetate succinate, such as AFFINISOL® HPMCAS 716 G (Dow Chemical), AFFINISOL® HPMCAS 912 G (Dow Chemical), and AFFINISOL® HPMCAS 126 G (Dow Chemical), polyvinyl acetate phthalate, such as PHTHALAVIN® (Berwind Pharmaceutical Services), methacrylic acid based copolymer, such as methacrylic acid-ethacrylate copolymer, such as EUDRAGIT® L100-55 (Evonik, Germany), and any combinations thereof.

The secondary excipient may be hydroxy propyl methyl cellulose acetate succinate. The hydroxy propyl methyl cellulose acetate succinate may have 5-14%, more particularly 10-14%, and more particularly 12% acetate substitution. The hydroxy propyl methyl cellulose acetate succinate may have 4-18%, more particularly 4-8%, more particularly 6% succinate substitution.

A polymer excipient may include only one polymer, or a pharmaceutical formulation of the present disclosure may include a combination of polymer excipients.

Any excipient, including any cyclic oligomer excipient or any polymer excipient, in a pharmaceutical formulation of the present disclosure may also not contain substantial levels of impurities. For example, the excipient in a pharmaceutical formulation of the present disclosure may be have less than 1.0%, 0.75%, 0.5%, 0.1%, 0.05%, or 0.01% impurities by weight as compared to total weight of excipient and impurities, relative to a standard of known concentration in mg/mL. Impurities may include excipient degradation products, such as thermal degradation products.

A pharmaceutical formulation of the present disclosure may also include one or more additives, such as one or more lipids. A pharmaceutical formulation of the present disclosure may be formulated using one or more lipid technologies. Lipids may be synthetic, semi-synthetic, or natural lipids. Lipids may be anionic, cationic, or neutral. Exemplary lipids include fats, fatty acids such as saturated, monounsaturated, polyunsaturated, omega-3, alpha-linolenic acid (ALA), eicosapentaenoic (EPA) and docosahexaenoic acid (DHA), omega-6, arachidonic acid (AA), linoleic acid, conjugated linoleic acid (CLA), and trans fatty acids, short-chain fatty acids (SCFAs) such as alpha-lipoic acid, medium-chain fatty acids (MCFAs), long-chain fatty acids (LCFAs), very long-chain fatty acids (VLCFAs), monoglycerides, diglycerides, triglycerides, phospholipids such as lecithin (phosphatidylcholine), sterols, cholesterol, phytosterols (plant sterols and stanols), carotenoids such as astaxanthin, lutein and zeaxanthin, lycopene, vitamin A-related carotenoids, waxes, and any combinations thereof.

A pharmaceutical formulation of the present disclosure may be formed by any suitable method. Inclusion complexes of the present disclosure may be formed by any suitable method, such as thermokinetic compounding, hot-melt extrusion, lyophilization, and/or spray drying, among other methods described herein. A pharmaceutical formulation of the present disclosure may be prepared using other methods including but not limited to wet mass extrusion, high intensity mixing, high intensity mixing with a solvent, ball milling, ball milling with a solvent, or any solvent casting or forming process with a high mixing step, among others identifiable by skilled persons upon reading the present disclosure.

Thermokinetic compounding may be particularly useful for forming inclusion complexes of APIs or excipients that experience degradation in hot melt extrusion or that do not have a common organic solvent system with the API as to facilitate spray drying.

As discussed above, thermokinetic compounding may also be particularly useful in obtaining increased yields of inclusion of APIs within the cyclic oligomers of the inclusion complexes, as compared to other methods of formulation. Without limitation to theory, thermokinetic compounding may provide increased compounding forces, such as increased shear forces, facilitating greater inclusion complexation of the API within the lipophilic interior of the cyclic oligomer than is possible using other formulation methods. Accordingly, thermokinetic compounding may provide a decrease in the amount of API that remains unincluded in available cyclic oligomer in an inclusion complex of the present disclosure after formulation, as compared to the amount of API that remains unincluded in available cyclic oligomer after formulation using other formulation methods. In some embodiments, unincluded API may form amorphous API dispersed within the pharmaceutical formulation as an amorphous solid dispersion. In some embodiments, thermokinetic compounding may provide an increase of up to 5%, 10%, 15%, 20%, 25%, 30%, 35%, 40%, 45%, 50%, 55%, 60%, 65%, 70%, 75%, 80%, 85%, 90%, 95%, or 100% more inclusion complex formation of the API included within the cyclic oligomer than may be achieved using other formulation methods. In some embodiments, thermokinetic compounding may provide an increase of up to 2-fold, 3-fold, 4-fold, 5-fold, 6-fold, 7-fold, 8-fold, 9-fold, 10-fold, or 20-fold more inclusion complex formation of the API included within the cyclic oligomer than may be achieved using other formulation methods. Accordingly, thermokinetic compounding may facilitate formulation of APIs in inclusion complexes with cyclic oligomers, at higher concentrations, while maintaining solubility and/or bioavailability, than is achievable using other formulation methods.

A pharmaceutical formulation of the present disclosure containing an inclusion complex of an API with a cyclic oligomer may be formulated for oral administration (e.g., oral capsules, oral tablets), parenteral administration (e.g., powders for suspension or solutions), topical dosage forms (e.g., creams or lotions), or for other routes of administration such as buccal, ophthalmic, or otic dosage forms.

In some embodiments, an API-cyclic oligomer inclusion complex that are formed using a method that includes a thermokinetic compounding process may have increased solubility as compared to a physical mixture of the API and the cyclic oligomer formed using a method that does not include a thermokinetic compounding process. For example, in Examples 2, 4, and 5, an inclusion complex of ITZ with HPBCD, RIV with HPBCD, or NIN with HPBCD and magnesium stearate, respectively, that was formed using a method that included a thermokinetic compounding process showed increased solubility as compared to an uncompounded physical mixture of the API and cyclic oligomer. In some embodiments, a thermokinetically compounded inclusion complex of an API and a cyclic oligomer may provide increased solubility as compared to the uncompounded API and cyclic oligomer, or as compared to an inclusion complex of the API and cyclic oligomer formed using a method that does not include a thermokinetic compounding process, when tested using an in vitro dissolution assay using any suitable medium, such as a suitable simulated gastrointestinal medium, such as those described herein, and others identifiable by skilled persons upon reading the present disclosure. In some embodiments, a thermokinetically compounded inclusion complex of an API and a cyclic oligomer may have an increased Cmax, increased area under drug dissolution versus time curve (AUDC), or both, as compared to the uncompounded API and cyclic oligomer, or as compared to an inclusion complex of the API and cyclic oligomer formed using a method that does not include a thermokinetic compounding process. In some embodiments, the in vitro dissolution Cₘₐₓ and/or AUDC of a thermokinetically compounded inclusion complex of an API and a cyclic oligomer may be increased by up to 10%, 20%, 30%, 40%, 50%, 60%, 70%, 80%, or 90%, or by up to 2-fold, 3-fold, 4-fold, 5-fold, 6-fold, 7-fold, 8-fold, 9-fold, 10-fold, or 20-fold, or more, as compared to the uncompounded API and cyclic oligomer, or as compared to an inclusion complex of the API and cyclic oligomer formed using a method that does not include a thermokinetic compounding process. Analysis of dissolution of the API in the medium may be performed by any suitable method identifiable by skilled persons such as those described herein, including without limitation, UV spectrophotometry methods and/or HPLC methods, among others.

A pharmaceutical formulation of the present disclosure containing an inclusion complex of an API with a cyclic oligomer may dissolve more readily in the gastro-intestinal tract of a patient than a pharmaceutical formulation containing neat crystalline API, as evidenced by dissolution in at least one of 0.01 N HCl and biorelevant media, such as: Simulated Gastric Fluid (SGF), Fasted State Simulated Intestinal Fluid (FaSSIF), or Fed State Simulated Intestinal Fluid (FeSSIF).

The pharmaceutical formulation may be in a final dosage form that modifies or extends the release of API. This may include an extended release, delayed release, and/or pulsatile release profiles and the like. The pharmaceutical formulation may be in a tablet dosage form including a hydrophilic matrix that forms a swollen hydrogel in the gastric environment. This formation of hydrogel is intended to (1) retain the tablet in the stomach and (2) retard the release of the API so as to provide a continuous release of the drug over a period of about 24 hours. More specifically, the final dosage form may be an extended release oral drug dosage form for releasing the inclusion complex including the API and the cyclic oligomer into the stomach, duodenum and small intestine of a patient, and includes: a single or a plurality of solid particles including the inclusion complex of the API and the cyclic oligomer dispersed within a polymer or a combination of polymers that (i) swells unrestrained dimensionally by imbibing water from gastric fluid to increase the size of the particles to promote gastric retention in the stomach of the patient in which the fasted/fed mode has been induced; (ii) gradually the inclusion complex of the API and the cyclic oligomer diffuses or the polymer erodes over a time period of hours, where the diffusion or erosion commences upon contact with the gastric fluid; wherein the inclusion complex of the API and the cyclic oligomer is vital for solubilization of the API upon diffusion or erosion; and (iii) releases the API to the stomach, duodenum and small intestine of the patient, as a result of the diffusion or polymeric erosion at a rate corresponding to the time period. Exemplary polymers include polyethylene oxides, alkyl substituted cellulose materials and combinations thereof, for example, high molecular weight polyethylene oxides and high molecular weight or viscosity hydroxypropylmethyl cellulose materials. An example polymer combination includes combination of polyethylene oxide POLYOX™ WSR 301 and hydroxypropyl methyl cellulose Methocel® E4M, used at ˜24% w/w and ˜18% w/w of the final tablet dosage form, respectively. This dosage from is intended to produce a pharmacokinetic profile with a reduced Cₘₐₓ-to-Cₘᵢₙ ratio such that human plasma concentrations remain within the therapeutic window for the duration of treatment. This API pharmacokinetic profile is expected to provide more efficacious therapeutic effect with similar or reduced side effects.

The example above is only one example by which one can achieve a prolonged release of the inclusion complex of the API and the cyclic oligomer and thereby minimize the Cₘₐₓ-to-Cₘᵢₙ ratio in a patient. Another example is a pulsatile release final dosage form containing a component designed to release the solubility enhanced inclusion complex of the API and the cyclic oligomer immediately in the stomach and one or more additional components designed to release a pulse of the inclusion complex of the API and the cyclic oligomer at different regions in the intestinal tract. This can be accomplished by applying a pH-sensitive coating to one or more components of the inclusion complex of the API and the cyclic oligomer—whereby the coating is designed to dissolve and release the active in different regions along the GI tract depending upon environmental pH. These functionally coated components may also contain an acidifying agent to decrease the microenvironmental pH to promote solubility and dissolution of the API.

Furthermore, there are a myriad of controlled release technologies that could be applied to generate an extended release profile of the inclusion complex of the API and the cyclic oligomer when starting from the pharmaceutical formulations including the inclusion complex of the API and the cyclic oligomer disclosed herein. It is important to note that the inclusion complex of the API and the cyclic oligomer is enabling to this approach as applying conventional controlled drug release technologies to crystalline APIs disclosed herein would fail to provide adequate API release along the GI tract owing to the poor solubility of these forms of the compound.

It is contemplated that selection of a final dosage form that modifies or extends the release of an API in a pharmaceutical formulation that includes an inclusion complex of a particular API and a cyclic oligomer may require screening and testing of a large number of candidate modified or extended release dosage forms. In particular, it is contemplated that selecting one or more suitable modified or extended release dosage forms that provide one or more possible performance enhancing effects of the pharmaceutical formulation in terms of stability, solubility, bioavailablility, and so on, will not be routine or predictable, but rather certain unpredictable combinations may provide surprising performance enhancing results.

In some embodiments, in a pharmaceutical formulation or inclusion complex of the present disclosure, the cyclic oligomer may be the only excipient. In some such embodiments, the pharmaceutical formulation or inclusion complex may include 1% to 50% by weight API, and between 50% and 99% by weight of one or more cyclic oligomer excipients. In some such embodiments, the pharmaceutical formulation or inclusion complex may include at least 5%, at least 10%, or at least 20% by weight API. In some such embodiments, the pharmaceutical formulation of inclusion complex may include at least 60% or at least 90% by weight of one or more cyclic oligomer excipients.

In some embodiments, in a pharmaceutical formulation or inclusion complex of the present disclosure, the cyclic oligomer may be the primary excipient. In some such embodiments, the pharmaceutical formulation or inclusion complex may include 1% to 50% by weight API, and between 50% and 99% by weight of one or more cyclic oligomer excipients. In some such embodiments, the pharmaceutical formulation or inclusion complex may include at least 5%, at least 10%, or at least 20% by weight API. In some such embodiments, the pharmaceutical formulation or inclusion complex may include at least 60% or at least 90% by weight of one or more cyclic oligomer excipients. The pharmaceutical formulation may further include at least 1% secondary excipient, such as a polymer secondary excipient.

In another pharmaceutical formulation or inclusion complex of the present disclosure, the cyclic oligomer may be the secondary excipient and the pharmaceutical formulation or inclusion complex may further include a primary excipient, such as a polymer primary excipient. In some such embodiments, in a pharmaceutical formulation or inclusion complex of the present disclosure, the cyclic oligomer may be the primary excipient. In some such embodiments, the pharmaceutical formulation or inclusion complex may include 1% to 50% by weight API, and between 50% and 99% by weight of one or more cyclic oligomer excipients. In some such embodiments, the pharmaceutical formulation or inclusion complex may include at least 5%, at least 10%, or at least 20% by weight API. In some such embodiments, the pharmaceutical formulation or inclusion complex may include at least 60% or at least 90% by weight of one or more cyclic oligomer excipients.

A pharmaceutical formulation of the present disclosure may include an amount of API in an inclusion complex with a cyclic oligomer sufficient to achieve the same or greater therapeutic effect, bioavailability, Cₘᵢₙ, Cₘₐₓ or Tₘₐₓ as a greater amount of amount of the API in crystalline form. An inclusion complex, or a pharmaceutical formulation including an inclusion complex as described herein may substantially improve the solubility of the API, which may facilitate the improvement in therapeutic effect, bioavailability, Cₘᵢₙ, Cₘₐₓ or Tₘₐₓ.

“Therapeutic effect” may be any measurable improvement in a patient over a course of treatment, such as a one-month course of treatment. Other scientifically accepted measures of therapeutic effect, such as those used in the course of obtaining regulatory approval, particularly FDA approval, may also be used to determine “therapeutic effect.”

For example, a therapeutic effect of itraconazole (ITZ) may include preventing or decreasing a fungal growth in a condition responsive to ITZ, such as those described herein. Specimens may be taken from patients for fungal cultures and other relevant laboratory studies (e.g., wet mount, histopathology, serology). Other measurements of therapeutic effect of ITZ may include in vitro or in vivo assays of cytochrome P450 inhibition.

For example, a therapeutic effect of sorafenib (SOR) may include decreasing tumor cell proliferation. Other measurements of therapeutic effect of ITZ may include in vitro or in vivo assays of inhibition of one or more intracellular (c-CRAF, BRAF and mutant BRAF) and cell surface kinases (KIT, FLT-3, RET, RET/PTC, VEGFR-1, VEGFR-2, VEGFR-3, and PDGFR-1B).

For example, a therapeutic effect of rivaroxaban (RIV) may include reducing or preventing the risk of stroke or systemic embolism, or deep vein thrombosis (DVT).

For example, a therapeutic effect of Nintedanib (NIN) may include reducing or preventing fibrosis.

“Bioavailability” may be measured as the area under the drug plasma concentration versus time curve (AUC) from an administered unit dosage form. Absolute bioavailability may refer to the bioavailability of an oral composition compared to an intravenous reference assumed to deliver 100% of the active into systemic circulation. In order to facilitate comparisons, bioavailability in the present disclosure may be measured on an empty stomach, such as at least two hours after the last ingestion of food and at least one hour before the next ingestion of food.

For example, the relative bioavailability of an API in a pharmaceutical formulation of the present disclosure as compared to the crystalline API may be at least 1.25-fold, 2-fold, 3-fold, 4-fold, 5-fold, 6-fold, 7-fold, 8-fold, 9-fold, 10-fold, or 20-fold greater.

A pharmaceutical formulation of the present disclosure may be for oral administration and may be further processed, with or without further compounding, to facilitate oral administration.

A pharmaceutical formulation of the present disclosure may be further processed into a solid dosage form suitable for oral administration, such as a tablet or capsule.

In order to further increase therapeutic effect, bioavailability, Cₘᵢₙ, or Cₘₐₓ of the API, a pharmaceutical formulation of the present disclosure may be combined with an additional amount of the primary excipient, secondary (or tertiary, etc.) excipient, such as hydroxy propyl methyl cellulose acetate secondary excipient, or another suitable concentration enhancing polymer not part of the pharmaceutical formulation to produce the solid dosage form.

It is contemplated that selection of a suitable concentration-enhancing polymer or an additional amount of the primary excipient, secondary (or tertiary, etc.) excipient for adding to a pharmaceutical formulation that includes an inclusion complex of a particular API and a cyclic oligomer may require screening and testing of a large number of candidate additional excipients. In particular, it is contemplated that selecting one or more suitable concentration-enhancing polymer or an additional amount of the primary excipient, secondary (or tertiary, etc.) excipient that provide one or more possible performance enhancing effects of the pharmaceutical formulation in terms of stability, solubility, bioavailablility, and so on, will not be routine or predictable, but rather certain unpredictable combinations may provide surprising performance enhancing results.

Concentration enhancing polymers suitable for use in the solid dosage form may include compositions that do not interact with the API in an adverse manner. The concentration enhancing polymer may be neutral or ionizable. The concentration enhancing polymer may have an aqueous solubility of at least 0.1 mg/mL over at least a portion of or all of pH range 1-8; particularly at least a portion of or all of pH range 1-7 or at least a portion of or all of pH range 7-8. When the solid dosage form is dissolved in in 0.01 N HCl and biorelevant media, such as: Simulated Gastric Fluid (SGF), Fasted State Simulated Intestinal Fluid (FaSSIF), or Fed State Simulated Intestinal Fluid (FeSSIF), the concentration-enhancing polymer may increase the maximum API concentration dissolved in the biorelevant media by a factor of at least 1.25, at least 2, or at least 3 as compared to an identical solid dosage form lacking the concentration enhancing polymer. A similar increase in maximum API concentration in biorelevant media may be observed when additional primary or secondary (or tertiary, etc.) excipients not present in the pharmaceutical formulation are added to the dosage form.

A pharmaceutical formulation of the present disclosure may be processed into a dosage form suitable for parenteral administration. As used herein, the term “parenteral” includes subcutaneous, intravenous (I.V.), intramuscular (I.M.), or infusion routes of administration. Dosage forms for parenteral administration may include suspension of the pharmaceutical formulation in a suitable carrier liquid, such as sterile saline (0.9% w/v), among others identifiable by skilled persons upon reading the present disclosure.

In some embodiments, the inclusion complexes and/or pharmaceutical formulations including inclusion complexes, of the present disclosure that are prepared by thermokinetic compounding provide advantages for oral or parenteral administration as compared to pharmaceutical formulations prepared using other methods. Without limitation to theory, it is expected that thermokinetic compounding facilitates higher efficiency of inclusion of APIs into inclusion complexes with cyclic oligomers as compared to other formulation methods.

Accordingly, in some embodiments, the inclusion complexes and/or pharmaceutical formulations including inclusion complexes, of the present disclosure that are prepared by thermokinetic compounding have higher concentrations of APIs that are included within cyclic oligomers in inclusion complexes as compared to inclusion complexes having a same molar ratio of cyclic oligomer:API that are formulated using other methods. Accordingly, the higher concentrations of APIs found in inclusion complexes and/or pharmaceutical formulations including inclusion complexes, of the present disclosure achievable using thermokinetic compounding is expected to provide increased therapeutic effect, bioavailability, Cₘᵢₙ, Cₘₐₓ, or any combinations thereof of the oral or parenteral formulation as compared to an oral or parenteral formulation prepared using other formulation methods.

### B. METHODS OF FORMULATING A PHARMACEUTICAL FORMULATION

A pharmaceutical formulation of the present disclosure, and, in particular, inclusion complexes of the pharmaceutical formulation, may be prepared by thermokinetic processing, also referred to herein as thermokinetic compounding, which is a method of compounding components until they are melt-blended. Thermokinetic compounding may be particularly useful for compounding heat-sensitive or thermolabile components. Thermokinetic compounding may provide brief processing times, low processing temperatures, high shear rates, and the ability to compound thermally incompatible materials.

Accordingly, in some embodiments, described herein is a method of making an inclusion complex by thermokinetic compounding, the inclusion complex comprising (i) one or more APIs described herein, or a pharmaceutically acceptable salt, ester, derivative, analog, prodrug, hydrate, or solvate thereof, or any combination thereof, wherein the API is not abiraterone or a pharmaceutically acceptable salt, ester, derivative, analog, prodrug, hydrate, or solvate thereof; and (ii) one or more cyclic oligomers described herein; wherein the method comprises thermokinetically processing the one or more APIs with one or more cyclic oligomers into the inclusion complex for less than 300 seconds.

Thermokinetic compounding may be carried out in a thermokinetic chamber using one or multiple speeds during a single, compounding operation on a batch of components to form and inclusion complex or a pharmaceutical formulation including an inclusion complex of the present disclosure.

A thermokinetic chamber includes a chamber having an inside surface and a shaft extending into or through the chamber. Extensions extend from the shaft into the chamber and may extend to near the inside surface of the chamber. The extensions are often rectangular in cross-section, such as in the shape of blades, and have facial portions. During thermokinetic compounding, the shaft is rotated causing the components being compounded, such as particles of the components being compounded, to impinge upon the inside surface of the chamber and upon facial portions of the extensions. The shear of this impingement causes comminution and/or frictional heating of the components and translates the rotational shaft energy into heating energy. Any heating energy generated during thermokinetic compounding is evolved from the mechanical energy input. Thermokinetic compounding is carried out without an external heat source. The thermokinetic chamber and components to be compounded are not pre-heated prior to commencement of thermokinetic compounding.

The thermokinetic chamber may include a temperature sensor to measure the temperature of the components or otherwise within the thermokinetic chamber.

During thermokinetic compounding, the average temperature of the thermokinetic chamber may increase to a pre-defined final temperature over the duration of the thermokinetic compounding to achieve thermokinetic compounding of the API and the excipient, and any other components of an inclusion complex or pharmaceutical formulation including an inclusion complex of the present disclosure, such as an additional API and/or an additional excipient. The pre-defined final temperature may be such that degradation of the API, excipient, or other components is avoided or minimized. Similarly, the one or multiple speeds of use during thermokinetic compounding may be such that thermal degradation of the API, excipient, or other components is avoided or minimized.

The average maximum temperature in the thermokinetic chamber during thermokinetic compounding may be less than the glass transition temperature, melting point, or molten transition point, of API or any other APIs present, one or all excipients, or one or all other components of the inclusion complex, or any combinations or sub-combinations of components.

Pressure, duration of thermokinetic compounding, and other environmental conditions such as pH, moisture, buffers, ionic strength of the components being mixed, and exposure to gasses, such as oxygen, may also be such that degradation of the API or any other APIs present, one or all excipients, or one or all other components is avoided or minimized.

Thermokinetic compounding may be performed in batches or in a semi-continuous fashion, depending on the product volume. When performed in a batch, semi-continuous, or continuous manufacturing process, each thermokinetic compounding step may occur for less than 5, 10, 15, 20, 25, 30, 35, 40, 45, 50, 55, 60, 70, 100, 120, 240, or 300 seconds.

Variations of thermokinetic compounding may be used depending on the inclusion complex and its components. For example, the thermokinetic chamber may be operated at a first speed to achieve a first process parameter, then operated at a second speed in the same thermokinetic compounding process to achieve a final process parameter. In other examples, the thermokinetic chamber may be operated at more than two speeds, or at only two speeds, but in more than two time internals, such as at a first speed, then at a second speed, then again at the first speed.

The API component may be in a crystalline or semi-crystalline form prior to thermokinetic compounding.

In another variation, the API or other API particle size may be reduced prior to thermokinetic compounding. This may be accomplished by milling, for example dry milling the crystalline form of the API or other API to a small particle size prior to thermokinetic compounding, wet milling the crystalline form of the API or other API with a pharmaceutically acceptable solvent to reduce the particle size prior to thermokinetic compounding, or melt milling the crystalline form of the API or other API with at least one excipient having limited miscibility with the crystalline form of the API or other API to reduce the particle size prior to thermokinetic compounding.

Another variation includes milling the crystalline form of the API or other API in the presence of an excipient to create an ordered mixture where the API or other API particles adhere to the surface of excipient particles, and/or excipient particles adhere to the surface of API particles.

The thermokinetically compounded inclusion complex may exhibit substantially complete amorphicity.

The inclusion complexes or pharmaceutical formulations including inclusion complexes of the present disclosure may be formulated without a solvent. For example, the inclusion complexes or pharmaceutical formulations including inclusion complexes of the present disclosure may be prepared using thermokinetic compounding without a solvent. Accordingly, an inclusion complex or pharmaceutical formulation including an inclusion complex of the present disclosure prepared by thermokinetic compounding may have no solvent in the inclusion complex, pharmaceutical formulation including the inclusion complex, or a tablet thereof, and may have no impurities including the solvent in the inclusion complex and/or pharmaceutical formulation including an inclusion complex and/or a tablet thereof.

In some embodiments, the inclusion complexes or pharmaceutical formulations including inclusion complexes of the present disclosure may be provided by a process that includes compounding the API and a cyclic oligomer excipient in a thermokinetic mixer at a temperature less than or equal to 200° C. for less than 300 seconds to form an inclusion complex of the API and the cyclic oligomer excipient.

In some embodiments, the present disclosure provides a method of forming an inclusion complex or a pharmaceutical formulation including an inclusion complex, the method including compounding (i) an API selected from Itraconazole (ITZ), Sorafenib (SOR), Rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, and ziprasidone, or a pharmaceutically acceptable salt, ester, derivative, analog, prodrug, hydrate, or solvate thereof, and any combinations thereof and (ii) a cyclic oligomer excipient in a thermokinetic mixer at a temperature less than or equal to 200° C. for less than 300 seconds to form an inclusion complex of the API and the cyclic oligomer excipient.

In some embodiments, the compounding in the thermokinetic mixer does not cause substantial thermal degradation of the API and/or substantial thermal degradation of the cyclic oligomer excipient.

In some embodiments, the compounding in the thermokinetic mixer provides a pharmaceutical formulation including an inclusion complex of the API and the cyclic oligomer excipient having an increase of up to 10%, 20%, 30%, 40%, 50%, 60%, 70%, 80%, 90% or more inclusion of the API within the cyclic oligomer as compared to a method that does not include a thermokinetic compounding process.

In some embodiments, the compounding in the thermokinetic mixer provides a pharmaceutical formulation including an inclusion complex of the API and the cyclic oligomer excipient having an increase of up to 2-fold, 3-fold, 4-fold, 5-fold, 6-fold, 7-fold, 8-fold, 9-fold, 10-fold, or 20-fold increase in inclusion of an API within a cyclic oligomer as compared to a method that does not include a thermokinetic compounding process.

In some embodiments, the present disclosure relates to a pharmaceutical formulation including an active pharmaceutical ingredient (API) and a cyclic oligomer excipient, wherein the pharmaceutical formulation is formed by a method including compounding the API and the cyclic oligomer excipient in a thermokinetic mixer at a temperature less than or equal to 200° C. for less than 300 seconds to form an inclusion complex of the API and the cyclic oligomer excipient.

In some embodiments, the present disclosure provides a pharmaceutical formulation including an active pharmaceutical ingredient (API) selected from Itraconazole (ITZ), Sorafenib (SOR), Rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, and ziprasidone, or a pharmaceutically acceptable salt, ester, derivative, analog, prodrug, hydrate, or solvate thereof, and any combinations thereof; and a cyclic oligomer excipient; wherein the pharmaceutical formulation is formed by a method including compounding the API and the cyclic oligomer excipient in a thermokinetic mixer at a temperature less than or equal to 200° C. for less than 300 seconds to form an inclusion complex of the API and the cyclic oligomer excipient. The disclosures of the following patents and patent applications are incorporated by reference in their entireties herein: U.S. Pat. No. 6,709,146, EP1365853B1, U.S. Pat. Nos. 8,486,423, 9,339,440, 6,073,043, PCT/US2008/073913, U.S. Pat. Nos. 10,022,385, 9,545,361, and PCT/US2014/034601.

For example, in some embodiments, thermokinetic compounding may be performed using a KinetiSol® (DisperSol Technologies, LLC, Texas) method of processing of the API and the cyclic oligomer (see Examples).

An inclusion complex in a pharmaceutical formulation of the present disclosure may be formed by methods other than thermokinetic compounding, but may be formed with decreased efficiency. For example, as described above, a method that does not include a thermokinetic compounding process may result in at least 10%, 20%, 30%, 40%, 50%, 60%, 70%, 80%, or 90% less inclusion of the API within the cyclic oligomer, for a same amount of API and cyclic oligomer. A method that does not include a thermokinetic compounding process may result in at least 2-fold, 3-fold, 4-fold, 5-fold, 6-fold, 7-fold, 8-fold, 9-fold, 10-fold, or 20-fold less inclusion of the API within the cyclic oligomer, for a same amount of API and cyclic oligomer.

A pharmaceutical formulation of the present disclosure may be formed using hot melt extrusion, whereby an excipient blend is heated to a molten state and subsequently forced through an orifice where the extruded product is formed into its final shape in which it is solidified upon cooling. The blend is conveyed through various heating zones typically by a screw mechanism. The screw or screws are rotated by a variable speed motor inside a cylindrical barrel where only a small gap exists between the outside diameter of the screw and the inside diameter of the barrel. In this conformation, high shear is created at the barrel wall and between the screw flights by which the various components of the powder blend are well mixed and deaggregated.

The hot-melt extrusion equipment is typically a single or twin-screw apparatus but can be composed of more than two screw elements. A typical hot-melt extrusion apparatus contains a mixing/conveying zone, a heating/melting zone, and a pumping zone in succession up to the orifice. In the mixing/conveying zone, the powder blends are mixed and aggregates are reduced to primary particles by the shear force between the screw elements and the barrel. In the heating/melting zone, the temperature is at or above the melting point or glass transition temperature of the thermal binder or binders in the blend such that the conveying solids become molten as they pass through the zone. A thermal binder in this context describes an inert excipient, typically a polymer, that is solid at ambient temperature, but becomes molten or semi-liquid when exposed to elevated heat or pressure. The thermal binder acts as the matrix in which the API and other APIs are dispersed, or the adhesive with which they are bound such that a continuous composite is formed at the outlet orifice. Once in a molten state, the homogenized blend is pumped to the orifice through another heating zone that maintains the molten state of the blend. At the orifice, the molten blend may be formed into strands, cylinders or films. The extrudate that exits is then solidified typically by an air-cooling process. Once solidified, the extrudate may then be further processed to form pellets, spheres, fine powder, tablets, and the like.

A pharmaceutical formulation as disclosed herein resulting from hot melt extrusion may have a uniform shape and density and may not exhibit substantially changed solubility or functionality of any excipient. The API, excipient, or other components of the pharmaceutical formulation may lack substantial impurities.

A pharmaceutical formulation of the present disclosure may be prepared using spray drying. In the spray-drying process, components, including API, an excipient and any other APIs or excipients are dissolved in a common solvent which dissolves the components to produce a mixture. After the components have been dissolved, the solvent is rapidly removed from the mixture by evaporation in the spray-drying apparatus, resulting in the formation of a composition. Rapid solvent removal is accomplished by either (1) maintaining the pressure in the spray-drying apparatus at a partial vacuum (e.g., 0.01 to 0.50 atm); (2) mixing the mixture with a warm drying gas; or (3) both (1) and (2). In addition, a portion or all of the heat required for solvent evaporation may be provided by heating the mixture.

Solvents suitable for spray-drying can be any organic compound in which the API and primary excipient and any additional APIs or excipients are mutually soluble. The solvent may also have a boiling point of 150° C. or less. In addition, the solvent should have relatively low toxicity and be removed from the composition to a level that is acceptable according to The International Committee on Harmonization (ICH) guidelines, which are incorporated by reference herein. A further processing step, such as tray-drying subsequent to the spray-drying process, may be used to remove solvent to a sufficiently low level.

Suitable solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethylacetamide or dimethylsulfoxide may also be used. Mixtures of solvents may also be used, as may mixtures with water as long as the API, excipient, and any other APIs or excipients in the pharmaceutical formulation are sufficiently soluble to allow spray-drying.

The API, excipient, or other components of a pharmaceutical formulation as disclosed herein resulting spray-drying may lack substantial impurities.

A pharmaceutical formulation of the present disclosure may be prepared using other methods including but not limited to wet mass extrusion, high intensity mixing, high intensity mixing with a solvent, ball milling, ball milling with a solvent, or any solvent casting or forming process with a high mixing step, among others identifiable by skilled persons upon reading the present disclosure. The API, excipient, or other components of a pharmaceutical formulation as disclosed herein provided by any of the methods described herein or identifiable by skilled persons upon reading the present disclosure may lack substantial impurities.

Following formulation of a pharmaceutical formulation as disclosed herein, an amount appropriate to provide a given unit dosage form may be further processed, for example to result in an orally administrable form or a parenterally administrable form.

For an oral dosage form, this further processing may include combining the pharmaceutical formulation as an internal phase with an external phase, if needed, along with tableting by a tableting press or encapsulation in a capsule. The external phase may include an additional amount of an excipient or a concentration enhancing polymer to further improve, for example, the therapeutic effect, bioavailability, Cₘᵢₙ, or Cₘₐₓ.

In some examples, the pharmaceutical formulation may be tableted, then coated with a composition containing another API.

### C. METHODS OF ADMINISTERING A PHARMACEUTICAL FORMULATION

Low oral bioavailability of some forms of APIs, such as crystalline forms of APIs, may be associated with poor clinical outcomes in a significant portion of the patient population.

For example, with regard to itraconazole (ITZ), SPORANOX® Capsules contain 100 mg of itraconazole. The observed absolute oral bioavailability of itraconazole is 55% (SPORANOX® Prescribing Information). The recommended daily dose of SPORANOX® is 200 to 400 mg. According to the SPORANOX® Prescribing Information, SPORANOX® Capsules should be taken with a full meal to ensure maximal absorption.

With regard to sorafenib (SOR), for example, after administration of NEXAVAR tablets, the mean relative bioavailability is 38-49% when compared to an oral solution (NEXAVAR® Prescribing Information). The recommended daily dose of NEXAVAR® is 400 mg (2×200 mg tablets) taken twice daily without food.

With regard to Rivaroxaban (RIV), for example, for patients with creatinine clearance (CrCl)>50 m/min, the recommended dose of XARELTO® is 20 mg taken orally once daily with the evening meal. For patients with CrCl 15 to 50 m/min, the recommended dose is 15 mg once daily with the evening meal. Each XARELTO® tablet contains 10 mg, 15 mg, or 20 mg of rivaroxaban. According to XARELTO® Prescribing Information, the absolute bioavailability of rivaroxaban is dose-dependent. For the 10 mg dose, it is estimated to be 80% to 100% and is not affected by food. XARELTO 10 mg tablets can be taken with or without food. The absolute bioavailability of rivaroxaban at a dose of 20 mg in the fasted state is approximately 66%. Coadministration of XARELTO with food increases the bioavailability of the 20 mg dose (mean AUC and Cₘₐₓ increasing by 39% and 76% respectively with food). XARELTO 15 mg and 20 mg tablets should be taken with the evening meal (XARELTO® Prescribing Information). Absorption of rivaroxaban is dependent on the site of drug release in the GI tract. A 29% and 56% decrease in AUC and Cₘₐₓ compared to tablet was reported when rivaroxaban granulate is released in the proximal small intestine. Exposure is further reduced when drug is released in the distal small intestine, or ascending colon. According to XARELTO® Prescribing Information, administration of rivaroxaban via a method that could deposit drug directly into the proximal small intestine (e.g., feeding tube) should be avoided, as it can result in reduced absorption and related drug exposure.

With regard to Nintedanib (NIN), for example, maximum plasma concentrations are reached approximately 2 to 4 hours after oral administration as a soft gelatin capsule under fed conditions. The absolute bioavailability of a 100 mg dose of OFEV® was 4.7% (90% CI: 3.62 to 6.08) in healthy volunteers. Absorption and bioavailability are decreased by transporter effects and substantial first-pass metabolism. After food intake, Nintedanib exposure increased by approximately 20% compared to administration under fasted conditions (90% CI: 95.3% to 152.5%) and absorption was delayed (median Tₘₐₓ fasted: 2.00 hours; fed: 3.98 hours), irrespective of food type. (OFEV® Prescribing Information).

For example, better therapeutic effect with API treatment may be associated with a higher plasma Cₘₐₓ, or higher steady state Cₘᵢₙ following administration.

Accordingly, in some implementations, a pharmaceutical formulation of the present disclosure may include an amount of API in an inclusion complex with a cyclic oligomer sufficient to achieve an increased Cₘₐₓ or steady state Cₘᵢₙ in a human patient as compared to other formulations of the API, including without limitation commercially available formulations. Accordingly, administration of a pharmaceutical formulation of the present disclosure may provide increased plasma levels of the API in patients. Administration of a pharmaceutical formulation including an inclusion complex of the present disclosure may be associated with an increase in response to the administered API, for example, a greater proportion of patients showing one or more therapeutic effects. A pharmaceutical formulation of the present disclosure may include an inclusion complex of an API with a cyclic oligomer, which may exhibit improved therapeutic effect, bioavailability, Cₘᵢₙ, Cₘₐₓ, and any combinations thereof, as compared to an equivalent amount of the crystalline API.

A pharmaceutical formulation including an inclusion complex of the present disclosure may be administered in a dosage form, such as a unit dosage form containing an amount of API sufficient and at a frequency sufficient to achieve a greater therapeutic effect, the same or greater bioavailability, the same or greater Cₘᵢₙ, or the same or greater Cₘₐₓ, and any combinations thereof, as an equivalent amount of crystalline API, administered at the same frequency.

Accordingly, in some implementations, administration of a pharmaceutical formulation including an inclusion complex of the present disclosure, for example in a unit dosage form, may provide an increase in Cₘₐₓ, Cₘᵢₙ, or AUC, and any combinations thereof of the API in a human patient of up to 1.25-fold, 2-fold, 3-fold, 4-fold, 5-fold, 6-fold, 7-fold, 8-fold, 9-fold, 10-fold, or 20-fold, or higher, as compared to an administration of an equivalent amount of crystalline API.

Administration of a pharmaceutical formulation including an inclusion complex of the present disclosure, for example in a unit dosage form, may result in at least a 5% decrease, at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in variability among patients with a response within two standard deviations of the average response in therapeutic effect, bioavailability, Cₘᵢₙ, Cₘₐₓ, and any combinations thereof as compared to an administration of an equivalent amount of crystalline API.

Administration of a pharmaceutical formulation including an inclusion complex of the present disclosure, for example in a unit dosage form, may result in at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in fasting-state vs. high fat meal variability in therapeutic effect, bioavailability, Cₘᵢₙ, Cₘₐₓ, and any combinations thereof as compared to an administration of an equivalent amount of crystalline API.

Administration of a pharmaceutical formulation including an inclusion complex of the present disclosure, for example in a unit dosage form, may be administered using fewer or smaller tablets or capsules than is possible with other formulations, which may increase patient compliance and decrease patient discomfort.

The above and other improvements may be due, at least in part, to improved solubility of the API when present in a pharmaceutical formulation including an inclusion complex as of the present disclosure, as compared to the solubility of crystalline API. In particular, it is expected that a pharmaceutical formulation provided by a process that includes thermokinetic compounding of the cyclic oligomer and the API to form an inclusion complex provides increased solubility of the API as compared to an equivalent amount of the pharmaceutical formulation formed using other methods.

Such a pharmaceutical formulation including an inclusion complex may be designed for once-daily administration. Such a pharmaceutical formulation including an inclusion complex may be designed for twice-daily administration. Such a formulation may be designed for three times-daily, four times-daily or more administration.

Variations of the above example pharmaceutical formulations including inclusion complexes and dosing regimens are possible. For example, amounts of API in a pharmaceutical formulation including an inclusion complex of the present disclosure may be varied based upon the intended administration schedule.

In general, a pharmaceutical formulation including an inclusion complex of the present disclosure may be used to administer any amount of an API to a patient on any schedule.

A pharmaceutical formulation including an inclusion complex of the present disclosure may be used to administer an amount of API to a patient on a variable schedule. For example such variable schedules may include, over a period of 28 days, any combination of daily administration frequencies such as once daily (QD), twice daily (BID), three times daily (TID) and four times daily (QID) on each of the 28 days in the period. For example such variable schedules may include a combination of different doses on different days within the 28 day period. Other combinations of daily administration frequencies and daily doses are identifiable by skilled persons upon reading the present disclosure.

In addition, any pharmaceutical formulation including an inclusion complex of the present disclosure may be co-administered with any other API, whether or not in the pharmaceutical formulation, that also treats a condition responsive to the API.

### D. EXAMPLES

The present examples are provided for illustrative purposes only. They are not intended to and should not be interpreted to encompass the full breadth of the disclosure.

Various compositions and instruments are identified by trade name in this application. All such trade names refer to the relevant composition or instrument as it existed as of the earliest filing date of this application, or the last date a product was sold commercially under such trade name, whichever is later. One of ordinary skill in the art will appreciate that variant compositions and instruments sold under the trade name at different times will typically also be suitable for the same uses.

### Example 1: Methods of Formulating and Analyzing Inclusion Complexes of APIs with Hydroxypropyl β Cyclodextrin (HPBCD)

The following materials were used. Hydroxy propyl β cyclodextrin (Kleptose® HPB, Roquette Freres Corporation, France) was purchased from Roquette America (USA). Microcrystalline cellulose (Avicel PH-102) was purchased from FMC Corporation (Pennsylvania, USA). Mannitol (Pearlitol 200SD) was purchased from Roquette America (USA). Crosslinked sodium carboxy methyl cellulose (Vivasol®, J. Rettenmaier & Sohne GmbH and Co. Germany) was purchased from JRS Pharma (New York, USA). Hypromellose acetate succinate HMP grade (Shin-Etsu AQOAT®, Shin-Etsu Chemical Co., Ltd. Japan) was purchased from Shin-Etsu (New Jersey, USA). Colloidal Silicon Dioxide (Aerosil® 200 P, Evonik Degussa GmbH, Germany) was purchased from Evonik Industries (New Jersey, USA). Magnesium Stearate was purchased from Peter Greven (Muenstereifel, Germany). Fasted state simulated intestinal fluid (FaSSIF) dissolution media was prepared using FaSSIF/FeSSIF/FaSSGF powder purchased from Biorelevant.com (Surrey, UK). Solvents used for HPLC analysis were of HPLC grade. All other chemicals and reagents used for dissolution and HPLC analysis were of ACS grade.

KinetiSol® Processing. Compositions of various APIs indicated herein with hydroxy propyl β cyclodextrin (HPBCD) with different drug loading were prepared using KinetiSol® technology. Initially, all compositions were prepared using research-scale compounder (“Formulator”) designed and manufactured by DisperSol Technologies LLC (Texas, USA). Later, compositions were prepared using manufacturing-scale compounder (“Manufacturing compounder”) designed and manufactured by DisperSol Technologies LLC (Texas, USA). Prior to compounding, the API and the oligomer HPBCD were accurately weighed, and thoroughly mixed to prepare physical mixtures (PM). The physical mixtures were charged into the KinetiSol® compounder chamber. Inside the chamber, a shaft with protruding blades was rotated at varying incremental speeds ranging from 500 rpm to 7000 rpm, without external heat addition, to impart frictional and shear forces to the sample material. The temperature of the material was monitored using an infrared probe. When the temperature of the sample material reached a value of 75-200° C. (with the particular temperature depending on the particular API), the material was rapidly ejected, collected, and pressed between two stainless steel plates to rapidly quench the sample.

Milling. The quenched material obtained after KinetiSol® processing was milled in small batches, using a lab scale rotor mill (IKA tube mill 100, IKA Works GmbH & Co. KG, Staufen, Germany). For milling, the fragments of quenched material were loaded into a 20 mL grinding chamber which was operated between 10000-20000 rpm grinding speed for 60 seconds. The milled material was subsequently passed through a #60 mesh screen (≤250 μm). Material retained above the screen i.e. >250 μm was cycled through the mill with the same parameters and this process of milling and sieving was repeated until all material passed through the screen. The resultant material (≤250 μm) was labeled as KinetiSol®-processed composition (KPC).

Melt-quenching API. In order to provide neat amorphous API reference sample for nuclear magnetic resonance spectroscopy and Raman spectroscopy, API was melt-quenched. A small quantity of API (<0.5 grams) was added to an open scintillation vial, and heated with a blow torch for a few seconds until entire quantity of API melted. The scintillation vial containing the API was immediately submerged into liquid nitrogen. When the intensity of nitrogen boiling subsided, the vial was transferred into a vacuum desiccator and vacuum was applied for about 2 hrs. After 2 hrs, the vacuum was released, and the vial was removed from the desiccator. The quenched API material was scraped from the vial, lightly ground using a mortar and pestle and sieved via a #60 mesh screen (≤250 μm). The melt quenched API was placed in the freezer until further use.

X-ray Diffraction. X-ray diffraction (XRD) analysis was conducted using Rigaku MiniFlex600 II (Rigaku Americas, Texas, USA) instrument equipped with a Cu-Kα radiation source generated at 40 kV and 15 mA. The API, KPCs and melt-quench API samples were loaded into an aluminum pan, leveled with a glass slide and analyzed in the 2-theta range between 2.5-40.0° while being spun. The step size was 0.02°, and the scanning rate was set to 5.0°/min. A D/teX high speed detector was used. The following additional instrument settings were used: Slit condition: variable+fixed slit system; soller (inc.): 5.0 deg; IHS: 10.0 mm; DS: 0.625 deg; SS: 8.0 mm; soller (rec.): 5.0 deg; RS: 13.0 mm (Open) and monochromatization: kb filter (×2). The data was collected using Miniflex Guidance software (Rigaku Corporation, Tokyo, Japan) and processed using PDXL2 software (Rigaku Corporation, Tokyo, Japan).

Stability Analysis. Stability analysis was performed at elevated temperatures (also referred to herein as “heating studies”). KPC samples were loaded into a scintillation vial and heated on a hot plate set at various API-specific temperatures described herein, e.g. for ITZ, SOR, NIN, or RIV, for the indicated period of time. The samples were then analyzed by XRD as stated above. After XRD analysis, samples were re-heated to a higher API-specific temperature in an attempt to force crystallization of unincluded amorphous drug. The re-heated samples were held at this temperature for 6 hours, cooled to room temperature, and re-analyzed by XRD.

Modulated Differential Scanning Calorimetry. Thermal analysis was conducted by modulated differential scanning calorimetry (mDSC) using differential scanning calorimeter model Q20 (TA Instruments, Delaware, USA) equipped with a refrigerated-based cooling system and an autosampler. The API and KPC samples were prepared by weighing 5-10 mg of the material and loading it into a Tzero pan. The pan was sealed with Tzero lid using a Tzero press. Following the sample equilibration at 30° C. for 5 min, the temperature was ramped at set ramp rate up to a final temperature with a set modulation frequency and amplitude. The ramp rate, final temperature, and modulation may be set to API-specific values. Nitrogen was used as the sample purge gas at a flow rate of 50 mL/min. The data were collected using TA Instrument Explorer software (TA Instruments, Delaware, USA) and processed using Universal Analysis software (TA Instruments, Delaware, USA).

Raman Spectroscopy. Raman spectroscopy may be conducted using e.g. HyperFlux™ PRO Plus (HFPP) Raman spectroscopy system (Tornado Spectral Systems, Ontario, Canada). The API, PM, KPCs and melt-quench API samples may be loaded on an aluminum stage. The samples may be subjected to a laser beam with an appropriate wavelength, e.g. of 785 nm and power of 200 mW. An appropriate spectral range is used, e.g. 200-3300 cm⁻¹. Multiple exposures, e.g. fifty exposures may be collected per spectrum and e.g. 3 spectra collected per sample. Exposure time of e.g. 100 ms may be employed. Cosmic ray removal and dark spectral correction may be enabled. The spectral data may be collected using e.g. SpectralSoft software (Tornado Spectral Systems, Ontario, Canada). The spectral data pre-processing and multivariate analysis may be done using e.g. Unscrambler X software (Camo Analytics, Oslo, Norway).

Additional Materials and Methods:

The following additional methods may be used for preparation and analysis of the inclusion complexes in the pharmaceutical formulations described herein.

HPLC Analysis. A stability-indicating high-performance liquid chromatography (HPLC) method may be used for chemical analysis of KPCs. API-specific values for HPLC analysis parameters can be determined by skilled persons without undue experimentation upon reading the present disclosure. Such API-specific parameters include, for example, type of HPLC column, type of mobile phases, flow rate, gradient profile, run time, column temperature, analysis wavelength, sample preparation, diluent, and sample filtration. These parameters are API-specific and an appropriate stability indicating method for the API should be used. Samples chromatography may be analyzed using suitable commercially-available programs, such as Chromeleon™ software, version 7.0 (ThermoFisher Scientific, Massachusetts, USA), among others identifiable by skilled persons.

Solid state Nuclear Magnetic Resonance Spectroscopy. One-dimensional (1D) ¹³C solid state Nuclear Magnetic Resonance Spectroscopy (ssNMR) may be conducted in order to confirm the presence of an inclusion complex. A cross polarization experiment may be conducted using an appropriate MAS probe. The contact time, relaxation delay, spin rate, may be set to values appropriate for the inclusion complex under investigation. An appropriate chemical shift reference standard such as adamantane, which has a resonance frequency of 38.48 ppm, may be used.

Two-dimensional (2D) ¹³C-¹H heteronuclear correlation (HETCOR) spectra may be acquired in order to assess points of contact between APIs and cyclodextrins within the inclusion complexes. Appropriate experimental temperatures and MAS frequencies may be utilized. The ¹³C-¹H HETCOR experiments may be carried out using approprate CP contact times and recycle delays.

Phase Solubility Analysis. Parameters of suitable API-specific biorelevant dissolution methods such as phase solubility analysis can be determined by skilled persons without undue experimentation upon reading the present disclosure. For example, and without limitation, phase solubility analysis may be conducted in two separate media such as 0.01N HCl (pH 2.0) and FaSSIF (prepared in 50 mMol Phosphate Buffer pH 6.8). Solutions of HPBCD ranging from 0 mg/mL to 600 mg/mL may be prepared in each media in scintillation vials. An excess of API may be added to each vial and the vials may be sonicated for 30 minutes and placed on a bench. Samples may be pulled from each vial at time points of 48 hrs and 7 days. The samples may be centrifuged using an ultracentrifuge (Eppendorf, Hamburg, Germany). The supernatants may be further diluted using the HPLC diluent and analyzed by HPLC method mentioned above to find the concentration of API.

In vitro Dissolution Study. Parameters of suitable API-specific biorelevant dissolution methods such as in vitro dissolution analysis can be determined by skilled persons without undue experimentation upon reading the present disclosure. For example, and without limitation, an in vitro non-sink, gastric transfer dissolution method may be used to analyze the dissolution of API and KPC. For dissolution analysis samples equivalent to 44.6 mg of API, may be loaded in an Erlenmeyer flask (dissolution vessel) containing 50 mL of 0.01N HCl (pH 2.0), placed in an incubator-shaker-Excella E24 (New Brunswick Scientific, New Jersey, USA) set to 37° C. and a rotational speed of 180 rpm. After 30 min, 50 mL of FaSSIF (prepared in 50 mMol Phosphate Buffer pH 6.8) may be added to the dissolution vessel. At pre-determined time points, samples may be drawn from the dissolution vessel and centrifuged using an ultracentrifuge (Eppendorf, Hamburg, Germany). The supernatants may be further diluted using the HPLC diluent and analyzed by HPLC method mentioned above. The area under drug dissolution curve (AUDC) may be calculated by the linear trapezoidal method.

### Example 2: Formulation of Inclusion Complexes of Itraconazole (ITZ) and Ydroxypropyl β Cyclodextrin (HPBCD) Using Thermokinetic Compounding

This example describes thermokinetic compounding formulations of itraconazole (ITZ, as the API) together with hydroxypropyl β cyclodextrin (HPBCD) and analyses of the resulting formulated compositions to show that inclusion complexes were formed.

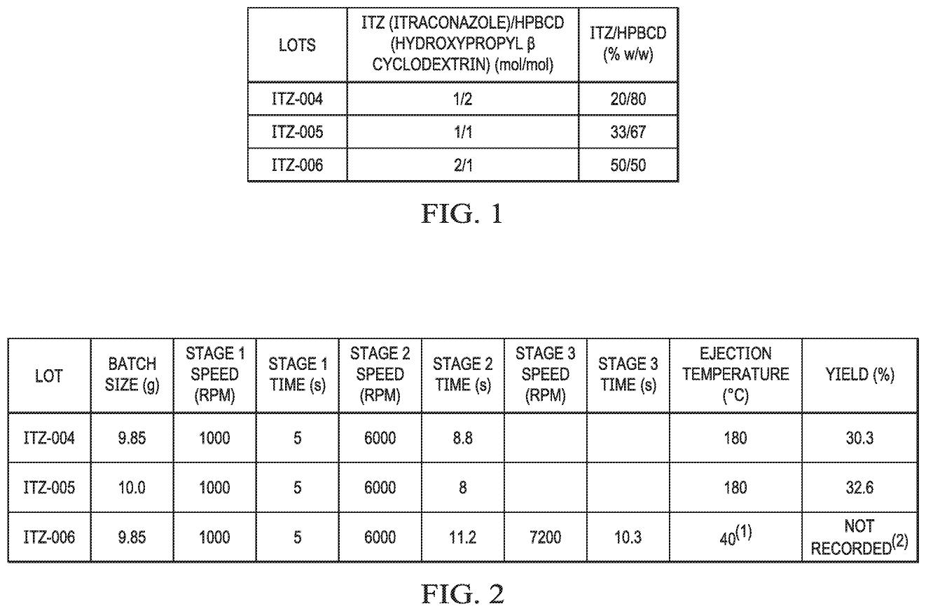

Itraconazole (ITZ) and hydroxypropyl β cyclodextrin (HPBCD) composition lots were prepared as shown in FIG. 1. Itraconazole and HPBCD raw powders were blended by hand in a suitable container for feeding into the formulator. The lots were processed by thermokinetic compounding as shown in FIG. 2 to provide KinetiSol®-processed composition (KPC) lots. In FIG. 2, superscript note (1) indicates second stage temperature peaked at 127° C. and began to drop; the stage was advanced manually. In FIG. 2, superscript note (2) indicates all material stuck to the inside of the chamber and was removed by scraping for further analysis.

After thermokinetic compounding, lot samples were processed in an IKA Tube Mill 100 at 25,000 rpm for 30 seconds to reduce particle size.

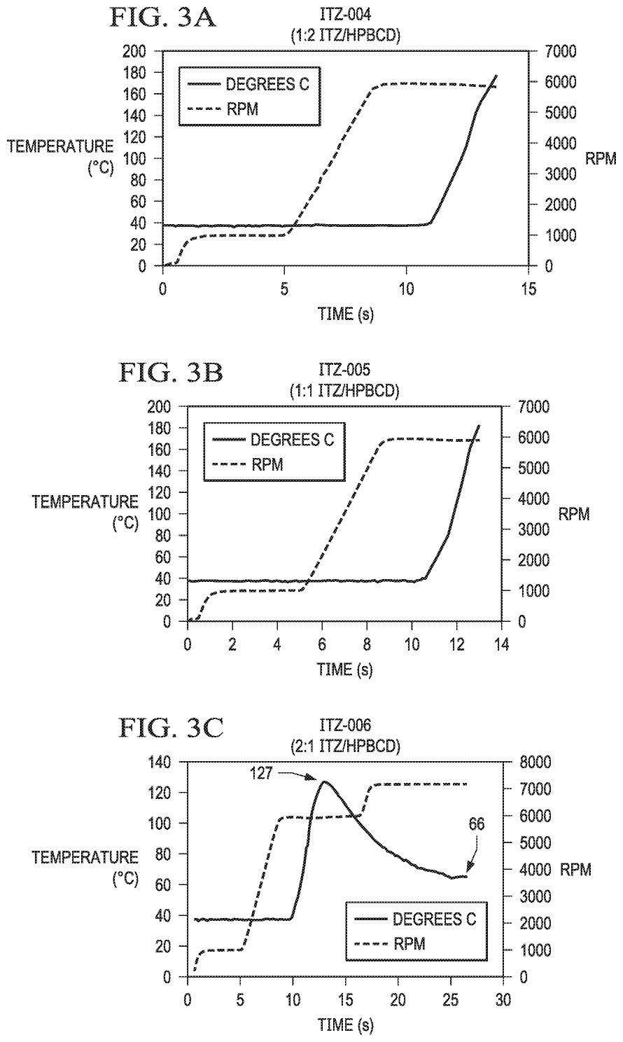

Thermokinetic compounding profiles. Results of thermokinetic compounding profiles for KinetiSol®-processed composition (KPC) lots ITZ-004, ITZ-005 and ITZ-006 are shown in FIG. 3A, FIG. 3B and FIG. 3C, respectively. The 1:2 and 1:1 (mol/mol) ITZ/HPBCD formulations were ejected at the set temperature of 180° C. The 2:1 ITZ/HPBCD formulation reached a peak temperature of 127° C., and stage advances and ejection occurred manually at 66° C.

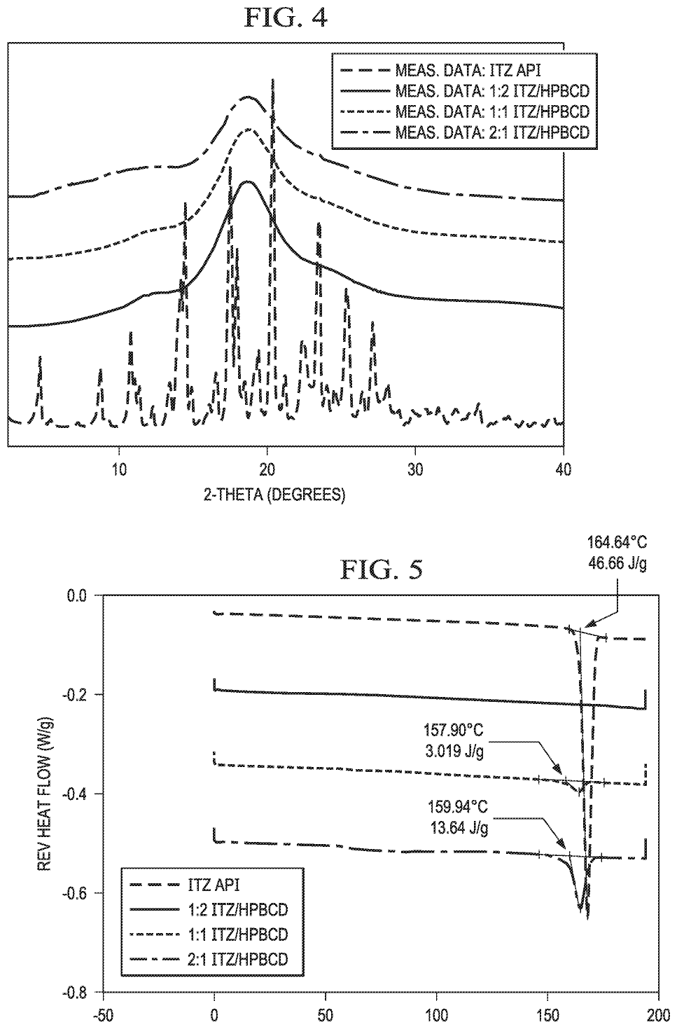

X-ray diffraction (XRD). X-ray diffraction (XRD) results for lots ITZ-004, ITZ-005 and ITZ-006, as well as crystalline ITZ API are shown in FIG. 4. FIG. 4 shows that the 1:2 formulation (lot ITZ-004) appeared completely amorphous by XRD, while the 2:1 formulation (lot ITZ-006) appeared trace crystalline.

Modulated differential scanning calorimetry (mDSC). Modulated differential scanning calorimetry (mDSC) results for lots ITZ-004, ITZ-005 and ITZ-006 and well as ITZ API (see FIG. 5) corroborated the XRD results. As shown in FIG. 5, no API melting endotherm is apparent for lot ITZ-004 at the 1:2 ITZ/HPBCD stoichiometry, whereas an API melting with an endotherm of 3.0 J/g occurred at 157.9° C. for lot ITZ-005 at the 1:1 ITZ/HPBCD stoichiometry and a melting endotherm of 13.6 J/g occurring at 159.9° C. for lot ITZ-006 at the 2:1 ITZ/HPBCD stoichiometry.

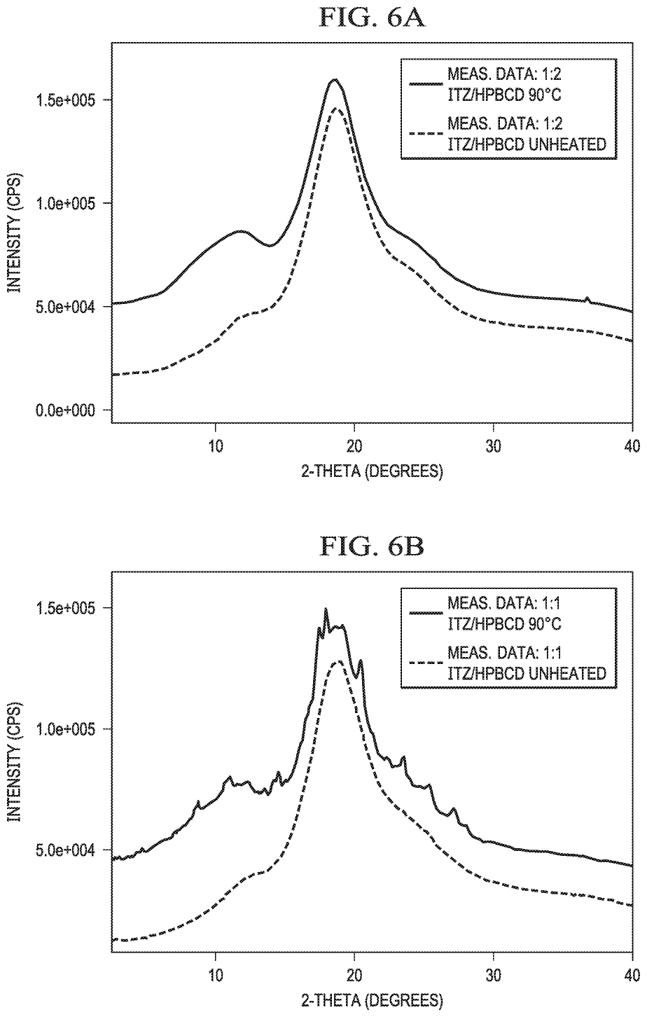

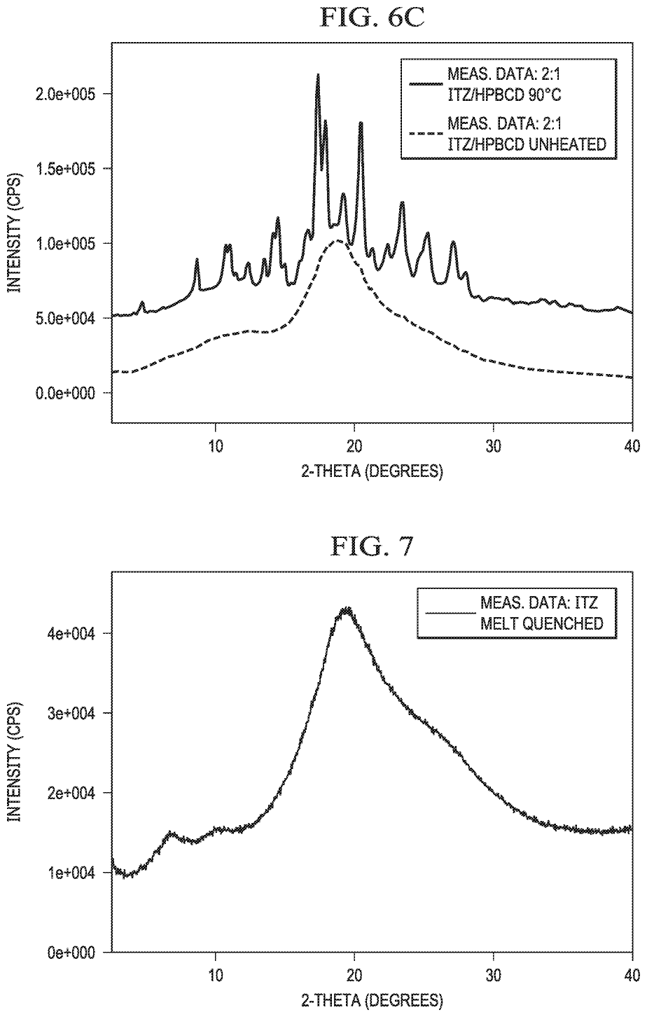

Heating studies: X-ray diffraction (XRD) of heated ITZ-HPBCD samples. Lot ITZ-004 (1:2 ITZ/HPBCD stoichiometry), lot ITZ-005 (1:1 ITZ/HPBCD stoichiometry), and lot ITZ-004 (2:1 ITZ/HPBCD stoichiometry) KPC's were heated to 90° C. for 6 hours, which is about 60° C. below the temperature of API melting onset. The heated KPC's were allowed to cool and analyzed by XRD. The results shown in FIG. 6A, FIG. 6B and FIG. 6C show that complexation with cyclodextrin confers stability against recrystallization of the API at elevated temperature.

As shown in FIG. 6A, 1:2 ITZ/HPBCD showed no recrystallization of the API after 15 hours at 90° C. by XRD, suggesting that itraconazole was completely incorporated into the hydrophobic cavity of the HPBCD at this stoichiometry. Complexation may occur between one ITZ molecule and two HPBCD, or it may be a mixture of 1:1 and 1:2 stoichiometries.

As shown in FIG. 6B, 1:1 ITZ/HPBCD showed some recrystallization of the API after 15 hours at 90° C. by XRD, suggesting that itraconazole was incompletely incorporated into the hydrophobic cavity of the HPBCD at this stoichiometry. The degree of recrystallization of ITZ was less for this KPC than the 2:1 formulation, however, demonstrating a greater incorporation of the ITZ into the cavity.

As shown in FIG. 6C, 2:1 ITZ/HPBCD showed substantial recrystallization of the API after 15 hours at 90° C. by XRD, suggesting that there was an excess of amorphous itraconazole that remained unincorporated in the hydrophobic cavity of the HPBCD. This amorphous, unincorporated ITZ was available for recrystallization at this stoichiometry.

Amorphous ITZ. Amorphous ITZ was prepared by melt-quenching for Raman spectral comparison with crystalline and HPBCD-included ITZ. Approximately 600 mg of ITZ were placed in a 20-mL scintillation vial. The vial was heated until the ITZ had completely melted. The vial containing the melt was submerged in liquid nitrogen to solidify the ITZ in its amorphous form. FIG. 7 is a graph reporting example XRD results for amorphous ITZ that was prepared by melt-quenching.

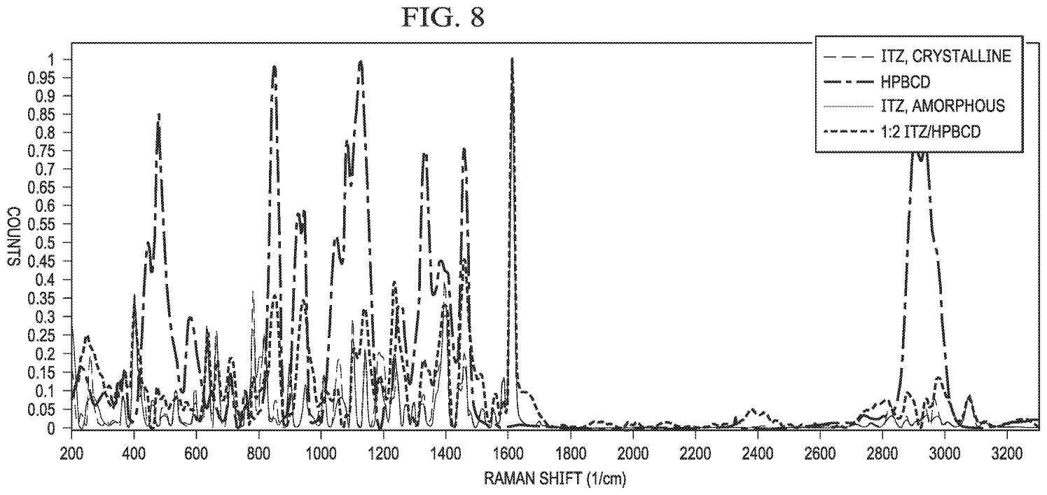

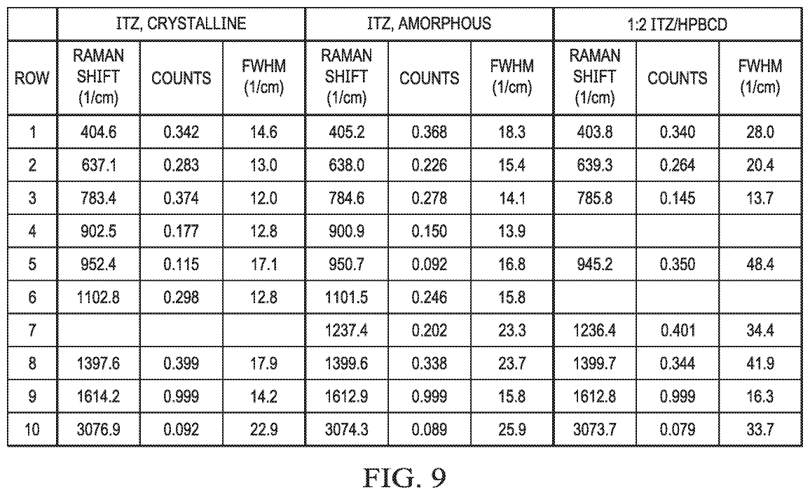

Raman spectroscopy. Raman spectroscopy was used to analyze crystalline ITZ, HPBCD, amorphous ITZ, and ITZ-004 (1:2 ITZ:HPBCD). FIG. 8 is a graph reporting example Raman spectra of crystalline ITZ, HPBCD, amorphous ITZ, and lot ITZ-004 (1:2 ITZ:HPBCD). Spectra shown are averages of three acquisitions. Spectral baseline corrections and peak normalizations have been applied. The HPBCD spectrum was subtracted from that of the ITZ-004 sample (the 1:2 ITZ/HPBCD KPC). FIG. 9 is a Table summarizing results of Raman spectral shift for crystalline ITZ, amorphous ITZ, and ITZ-004 (1:2 ITZ:HPBCD).

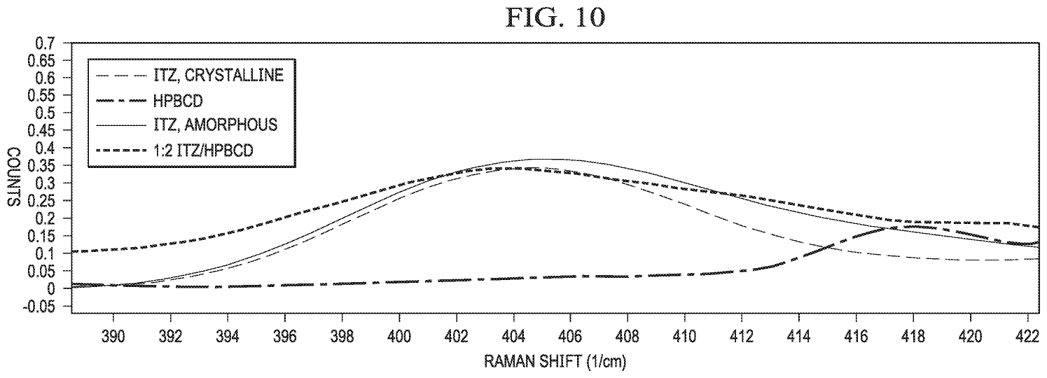

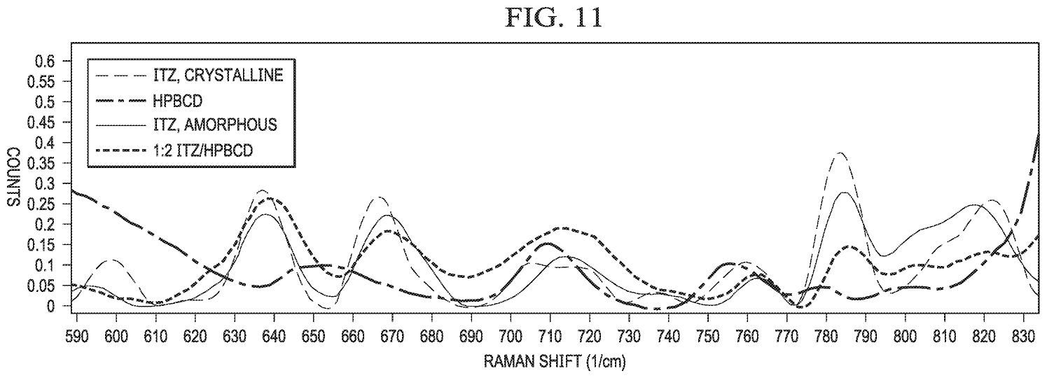

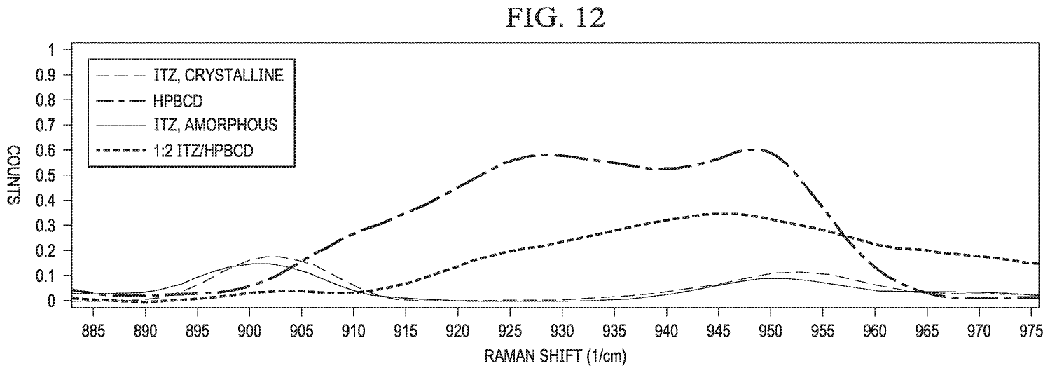

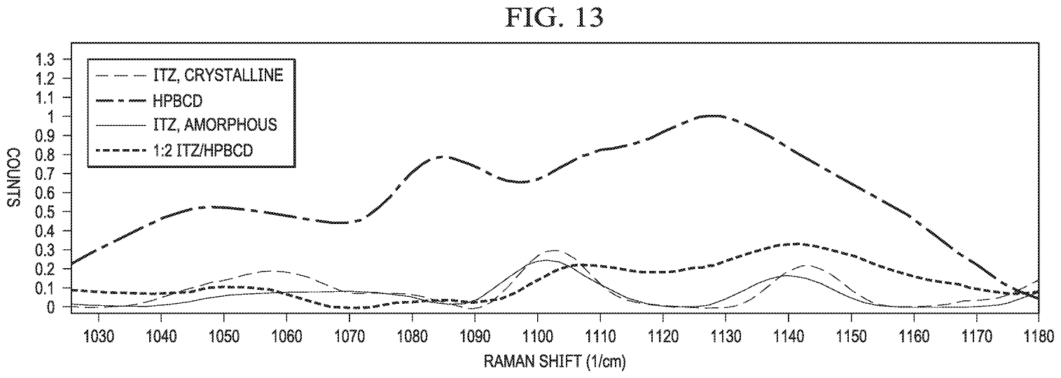

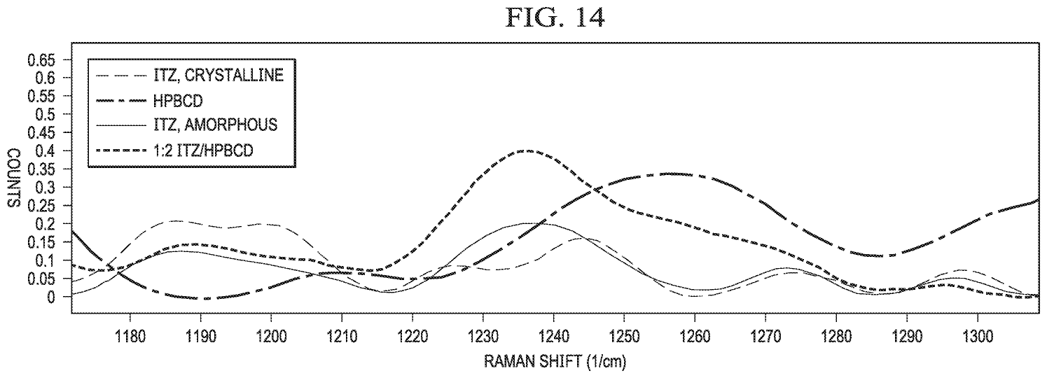

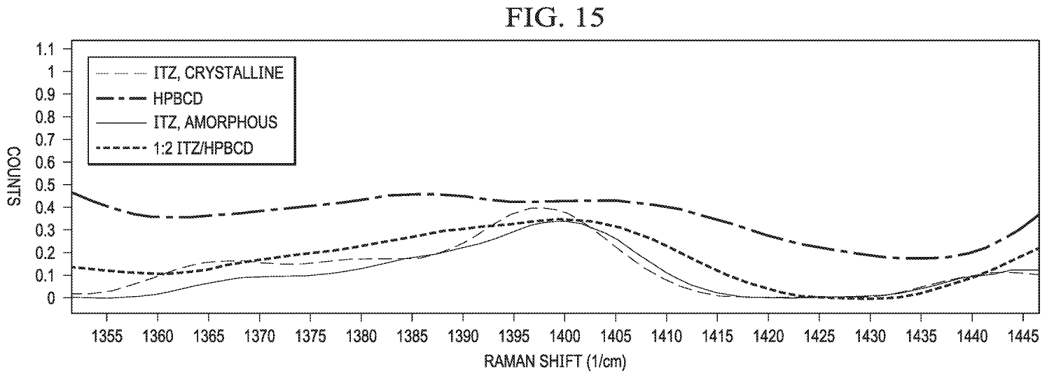

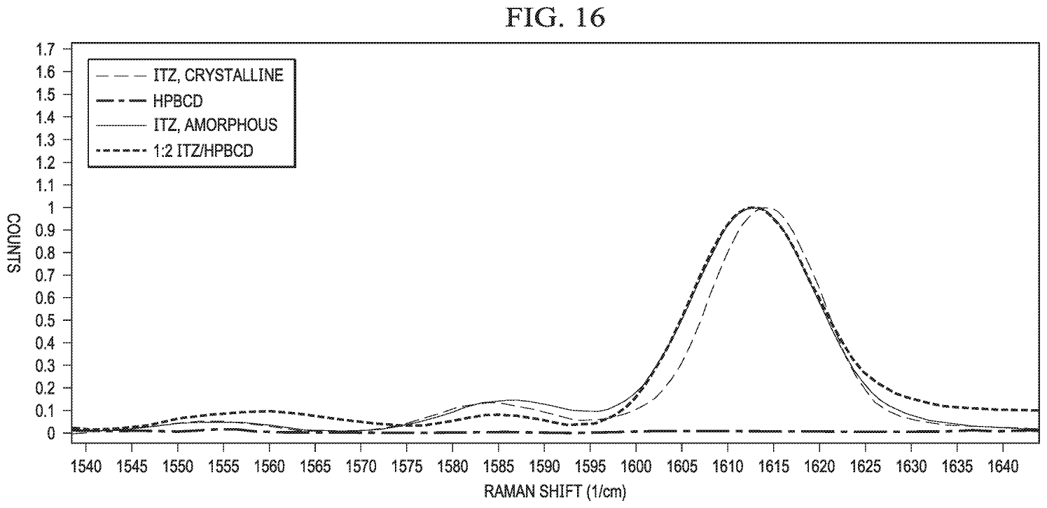

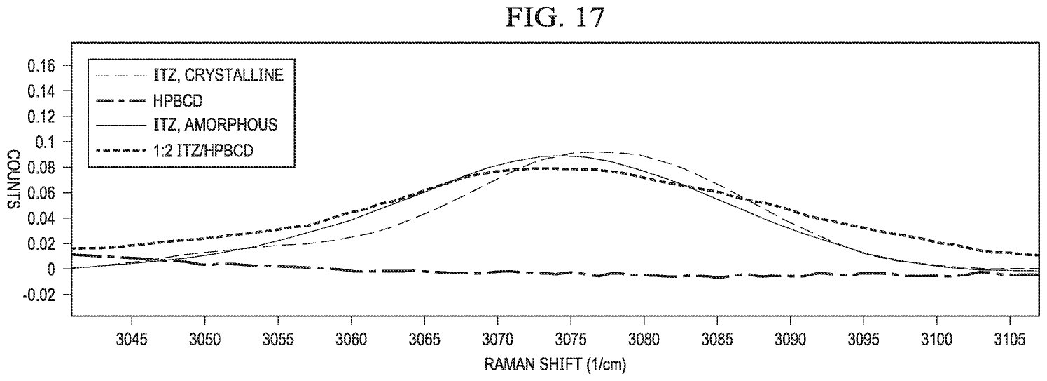

Comparing results across columns of the crystalline ITZ, HPBCD, amorphous ITZ, and the ITZ-004 KPC, for each of Rows 1-10 indicated in FIG. 9, the following observations are made. For data shown in Row 1, the ITZ-004 KPC sample showed peak broadening, and a shift to a lower wavenumber. Details of the spectral shift of Row 1 are shown in FIG. 10. For data shown in Row 2, the ITZ-004 KPC sample showed peak broadening, and a shift to a higher wavenumber. For data shown in Row 3, the ITZ-004 KPC sample showed a shift to a higher wavenumber. Details of the spectral shifts of Rows 2 and 3 are shown in FIG. 11. For data shown in Row 4, the ITZ-004 KPC sample peak was no longer detectable. For data shown in Row 5, the ITZ-004 KPC sample showed peak broadening, and possibly shifted to a lower wavenumber. Details of the spectral shifts of Rows 4 and 5 are shown in FIG. 12. For Row 6, the ITZ-004 KPC sample peak was shifted possibly to higher wavenumber. Details of the spectral shifts of Row 6 are shown in FIG. 13. For Row 7, two ITZ crystalline peaks have merged to a single broad peak. Details of the spectral shifts of Row 7 are shown in FIG. 14. For Row 8, the ITZ-004 KPC sample peak broadened and shifted. Details of the spectral shifts of Row 8 are shown in FIG. 15. For Row 9, the ITZ-004 KPC sample and amorphous ITZ sample peaks shifted to lower wavenumbers as compared to crystalline ITZ. Details of the spectral shifts of Row 9 are shown in FIG. 16. For Row 10, the ITZ-004 KPC sample showed peak broadening, and a shift to a lower wavenumber. Details of the spectral shifts of Row 10 are shown in FIG. 17.

In vitro Dissolution Study. An in vitro dissolution method was used to analyze the dissolution of the ITZ-004 (1:2 ITZ:HPBCD) physical mixture (PM) and a KPC thereof. The USP II apparatus was used in Paddle test configuration. The aqueous solution test medium was 750 mL of 0.1N HCl, pH 1.1. For dissolution analysis, samples equivalent to 37.5 mg of API for each of the ITZ-004 (1:2 ITZ:HPBCD) physical mixture (PM) and a KPC thereof (n=3 per sample) were added to the surface of the medium at the initiation of the test. The apparatus was set to 37° C. and a rotational speed of 75 rpm. After 1 hour and 2 hours, 5 mL samples were be taken from the medium and filtered through a 0.2 μm PVDF syringe filter. The filtered sample was used for analysis by HPLC

HPLC analysis. The in vitro dissolution samples were analyzed by HPLC using a Dionex Ultimate 3000 HPLC system (ThermoFisher Scientific, Massachusetts, USA). The HPLC column was a Kinetex® XB C18, 150 mm×4.6 mm, 2.6 μm (Phenomenex, California, USA). An isocratic mobile phase of 70:30:0.5 acetonitrile:H₂O:diethanolamine was used. The flow rate was 1.0 mL/min and the run time was 10 minutes. The data were collected at 263 nm. Samples chromatography was analyzed using Chromeleon™ software, version 7.0 (ThermoFisher Scientific, Massachusetts, USA)

The area under drug dissolution curve (AUDC) was calculated by the linear trapezoidal method.

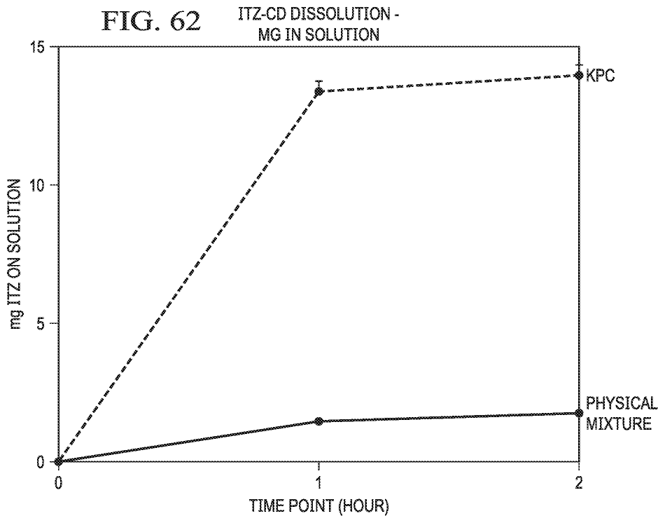

As shown in FIG. 62, thermokinetic compounding of the 1:2 ITZ:HPBCD formulation increased the AUDC observed from time 0 to time 1 hour by 9.1-fold as compared to the uncompounded physical mixture, and increased the AUDC observed from time 0 to time 2 hours by 8.7-fold as compared to the physical mixture.

Conclusions. In summary, modulated differential scanning calorimetry (mDSC), heating studies, and X-ray diffraction (XRD) results support the conclusion that the ITZ is included completely or partially in the relatively hydrophobic cavity of HPBCD when the ITZ/HPBCD molar ratios are appropriate (1:1 ITZ/HPBCD, 1:2 ITZ/HPBCD) in KPCs. Thus, inclusion complexes of ITZ API with HPBCD cyclic oligomer were formed.

In some embodiments, the inclusion complex formed may have a ratio of 1:1 or 1:2 ITZ/HPBCD, and complexation is incomplete in the absence of excess HPBCD.

Additional evaluations of the Raman spectroscopic evidence for which portions of the ITZ molecules are interacting with HPBCD may be determined using vibrational spectroscopic analysis of the Raman peak assignments. These additional analyses are expected to be in agreement with the results and conclusions described in this Example.

Thermokinetic compounding of 1:2 ITZ:HPBCD increased solubility of the ITZ as compared to 1:2 ITZ:HPBCD physical mixture (PM) in in vitro dissolution studies.

### Example 3: Formulation of Inclusion Complexes of Sorafenib (SOR) and Hydroxypropyl β Cyclodextrin (HPBCD) Using Thermokinetic Compounding

This example describes thermokinetic compounding formulations of Sorafenib (SOR, as the API) together with hydroxypropyl β cyclodextrin (HPBCD) and analyses of the resulting formulated compositions to show formation of inclusion complexes.

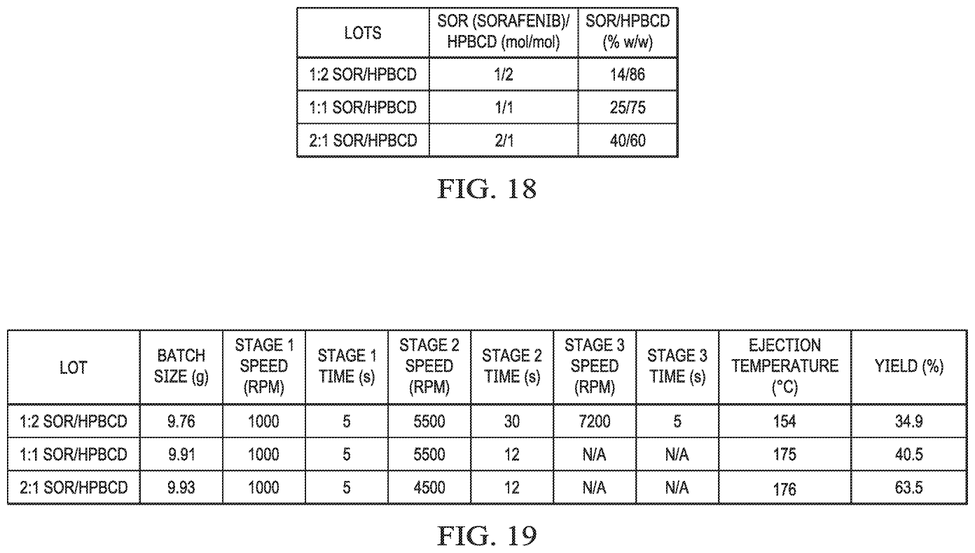

Sorafenib (SOR) and hydroxypropyl β cyclodextrin (HPBCD) composition lots were prepared as shown in FIG. 18. Sorafenib and HPBCD raw powders were blended by hand in a suitable container for feeding into the formulator. The lots were processed by thermokinetic compounding as shown in FIG. 19 to provide KinetiSol®-processed composition (KPC) lots. After thermokinetic compounding, lot samples were processed in an IKA Tube Mill 100 at 25,000 rpm for 30 seconds to reduce particle size.

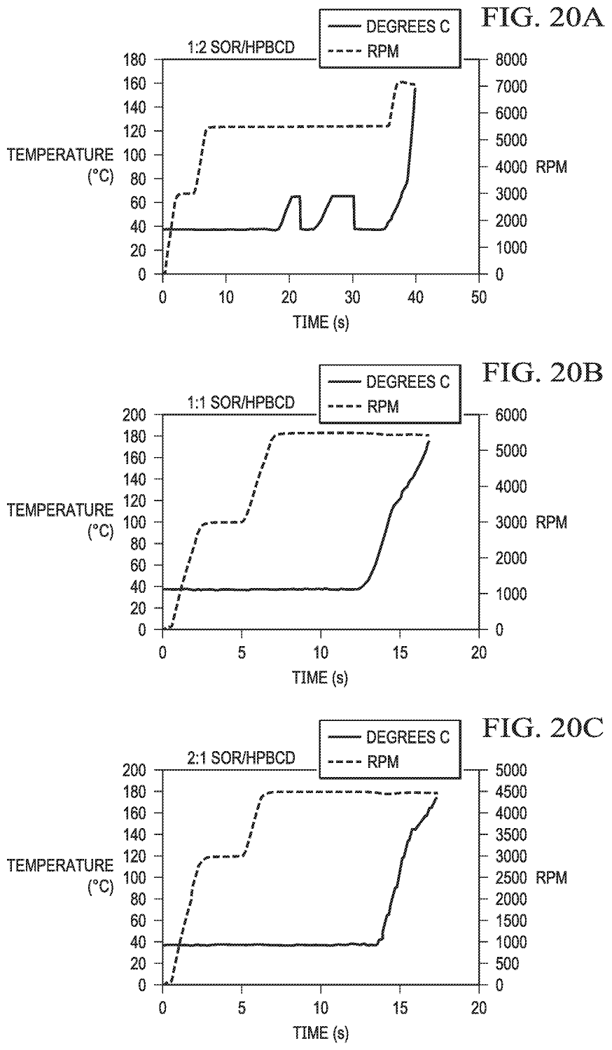

Thermokinetic compounding profiles. Results of thermokinetic compounding profiles for KinetiSol®-processed composition (KPC) lots 1:2 SOR/HPBCD, 1:1 SOR/HPBCD and 2:1 SOR/HPBCD are shown in FIG. 20A, FIG. 20B and FIG. 20C, respectively.

All three KPC lots were ejected at the programmed temperatures (160° C. for 1:2 and 180° C. for 1:1 and 2:1).

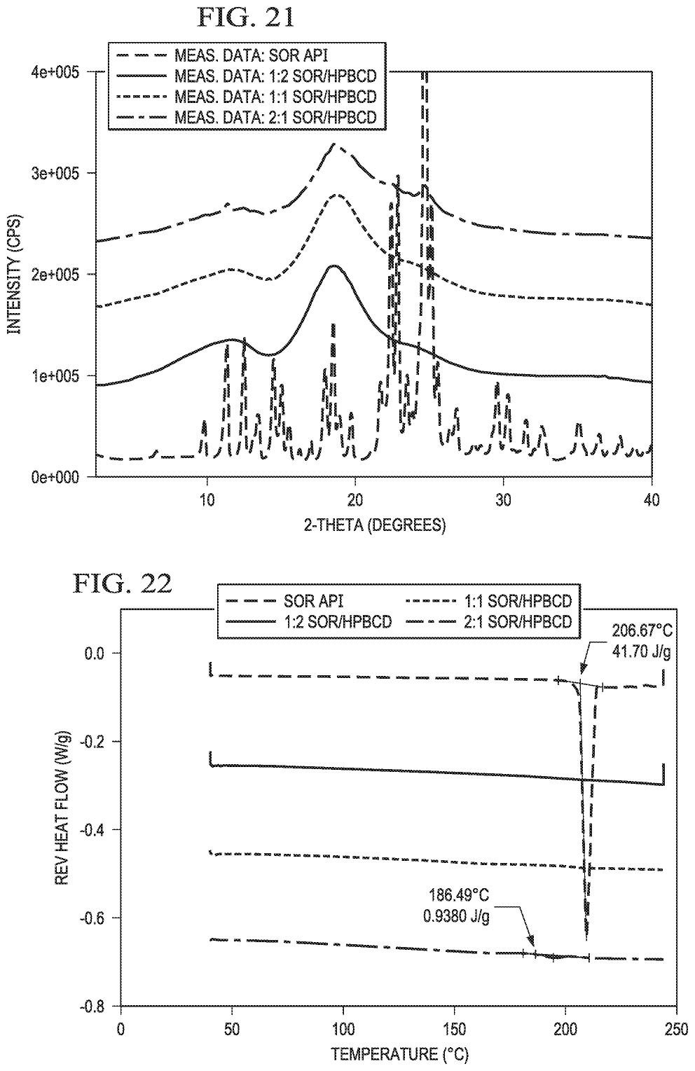

X-ray diffraction (XRD). X-ray diffraction (XRD) results for lots 1:2 SOR/HPBCD, 1:1 SOR/HPBCD and 2:1 SOR/HPBCD, as well as crystalline SOR API are shown in FIG. 21. FIG. 21 shows that the 1:2 and 1:1 SOR/HPBCD complexes were fully amorphous by XRD, while the 2:1 SOR/HPBCD complex showed some crystalline peaks.

Modulated differential scanning calorimetry (mDSC). Modulated differential scanning calorimetry (mDSC) results for lots 1:2 SOR/HPBCD, 1:1 SOR/HPBCD and 2:1 SOR/HPBCD as well as SOR API (see FIG. 22) corroborated the XRD results. As shown in FIG. 22, at the 1:2 and 1:1 SOR/HPBCD ratios, the SOR appears to be completely included in the HPBCD and only the 2:1 SOR/HPBCD had a detectable melting endotherm (0.938 J/g at 186° C.).

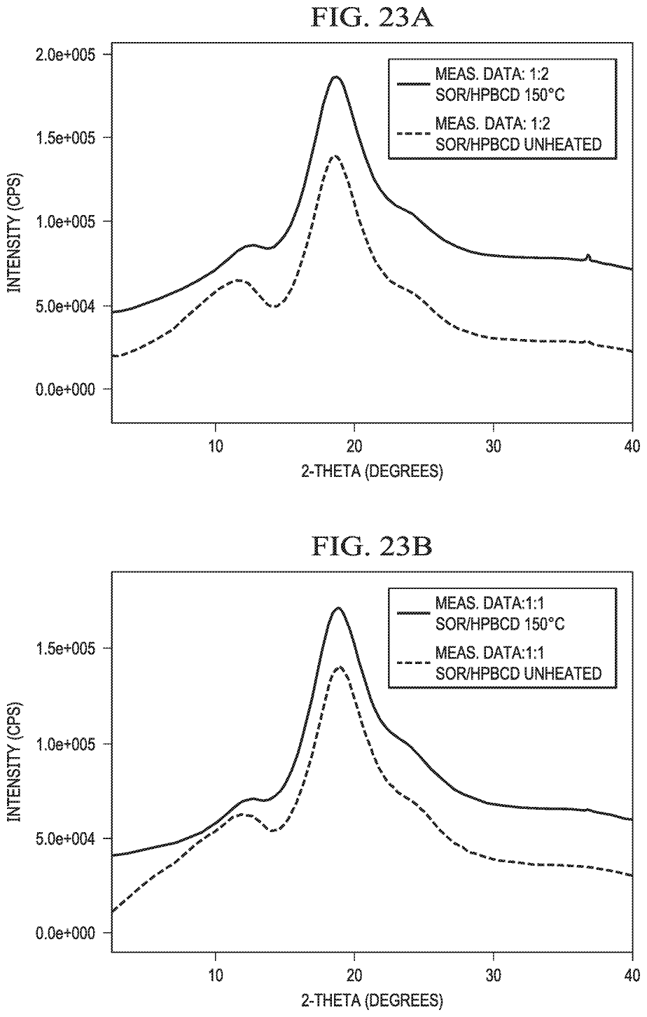

Heating studies: X-ray diffraction (XRD) of heated SOR-HPBCD samples. Lots 1:2 SOR/HPBCD, 1:1 SOR/HPBCD and 2:1 SOR/HPBCD KPCs were heated to 150° C. for 6 hours. The heated KPC's were allowed to cool and analyzed by XRD. The results shown in FIG. 23A, FIG. 23B and FIG. 23C show that complexation with cyclodextrin confers stability against recrystallization of the API at elevated temperature.

As shown in FIG. 23A, the 1:2 SOR/HPBCD KPC remained amorphous on heating, consistent with either a 1:1 or a 1:2 incorporation of SOR into the HPBCD cavities.

As shown in FIG. 23B, the 1:1 SOR/HPBCD KPC remained amorphous on heating, consistent with 1:1 incorporation of SOR into the HPBCD cavities.

As shown in FIG. 23C, the 2:1 SOR/HPBCD KPC had excess SOR which was unavailable for complexation. On heating, the crystalline peaks in the XRD grew more pronounced. This is consistent with the expectation that a fraction of the SOR was amorphous and unincluded in the HPBCD after compounding.

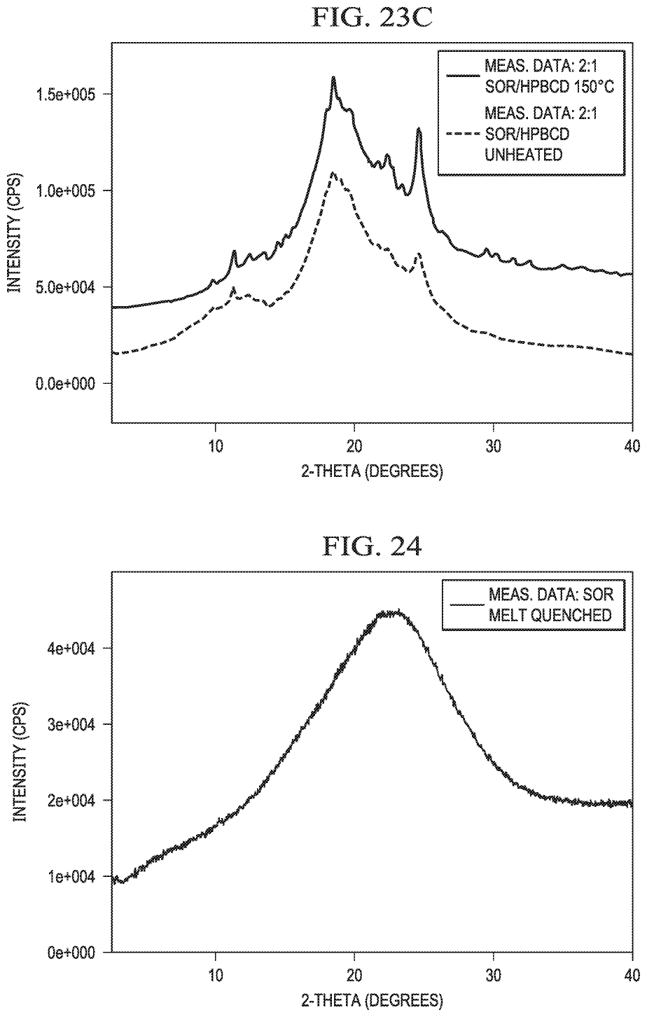

Amorphous SOR. Amorphous SOR was prepared by melt-quenching for Raman spectral comparison with crystalline and HPBCD-included SOR. Approximately 600 mg of SOR were placed in a 20-mL scintillation vial. The vial was heated until the SOR had completely melted. The vial containing the melt was submerged in liquid nitrogen to solidify the SOR in its amorphous form. FIG. 24 is a graph reporting example XRD results for amorphous SOR that was prepared by melt-quenching.

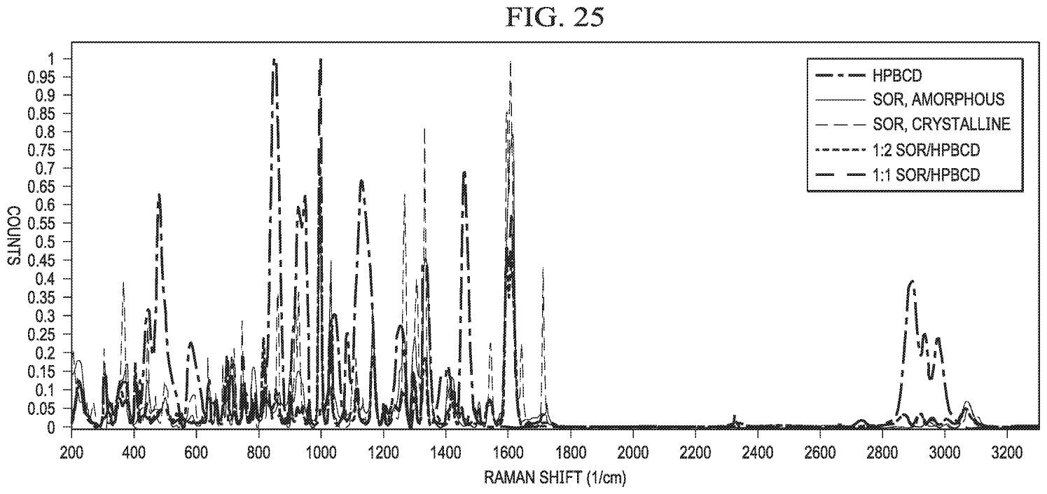

Raman spectroscopy. Raman spectroscopy was used to analyze crystalline SOR, HPBCD, amorphous SOR, and KPCs of 1:2 SOR/HPBCD and 1:1 SOR/HPBCD. FIG. 25 is a graph reporting an examplary Raman spectra of crystalline SOR, HPBCD, amorphous SOR, and KPCs of 1:2 SOR/HPBCD and 1:1 SOR/HPBCD. Spectra shown are averages of three acquisitions. Spectral baseline corrections and peak normalizations have been applied. The HPBCD spectrum was subtracted from that of the SOR/HPBCD KPCs.

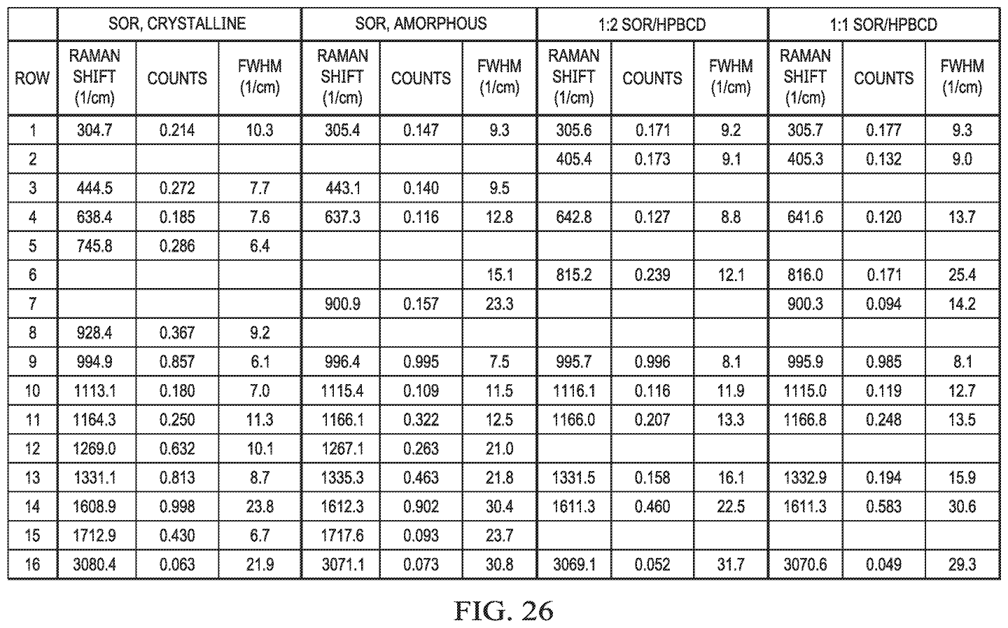

FIG. 26 is a Table summarizing results of Raman spectral shift for crystalline SOR, HPBCD, amorphous SOR, and KPCs of 1:2 SOR/HPBCD and 1:1 SOR/HPBCD.

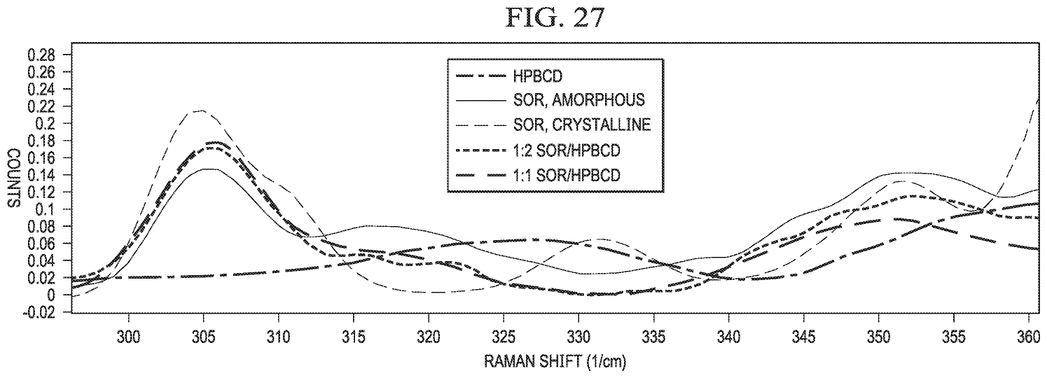

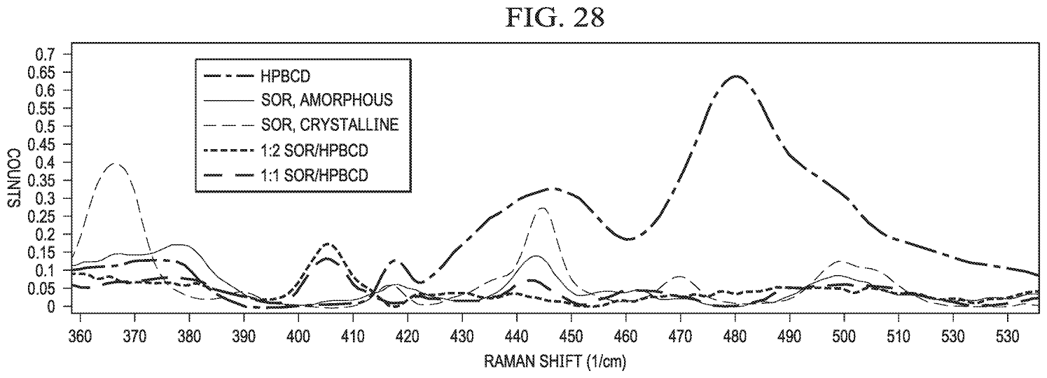

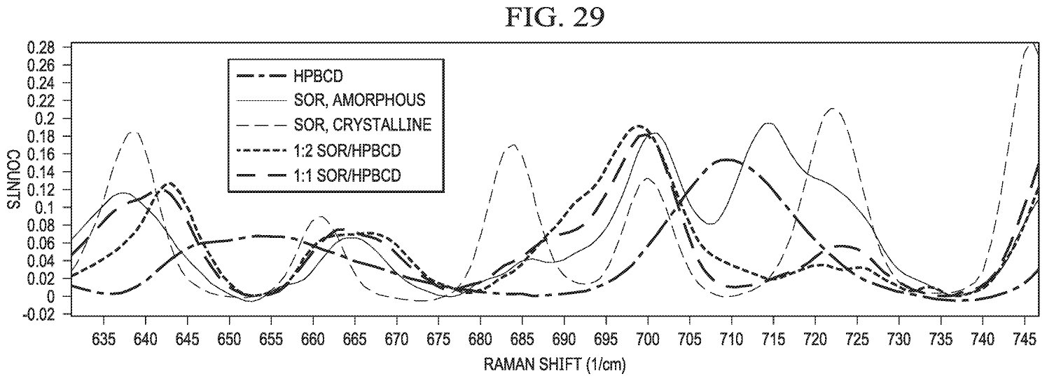

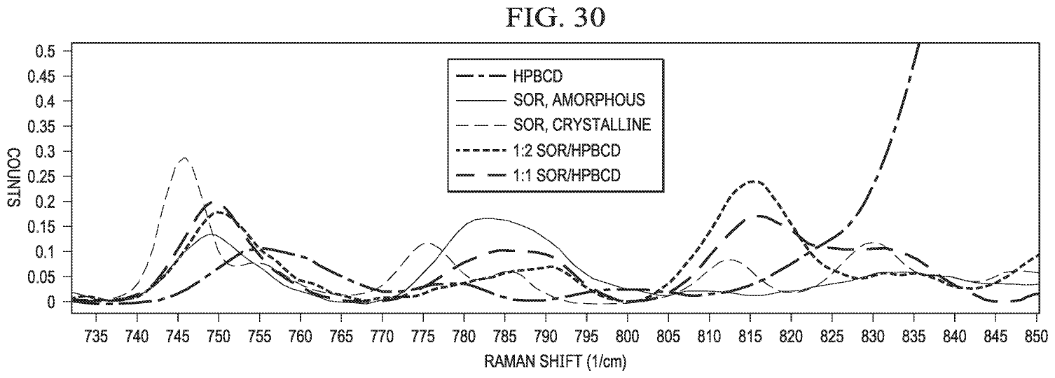

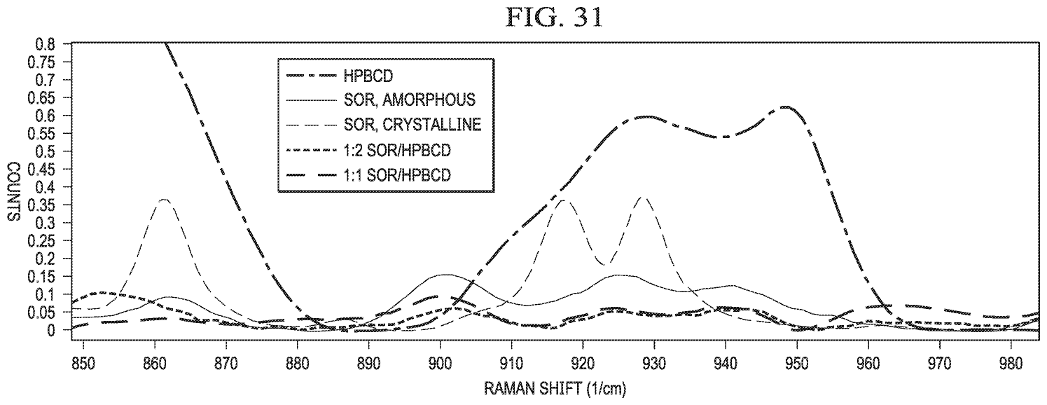

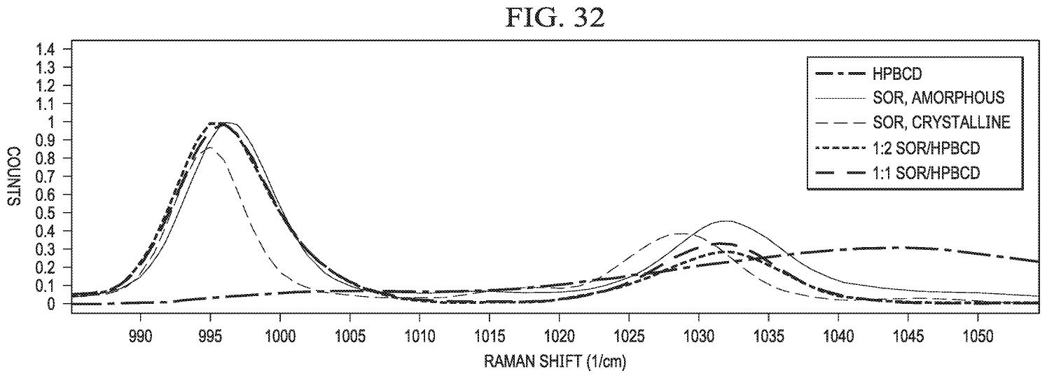

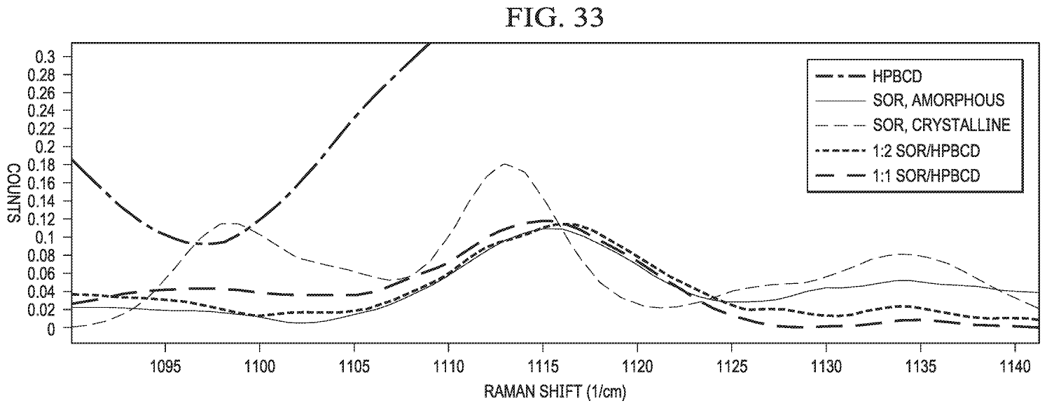

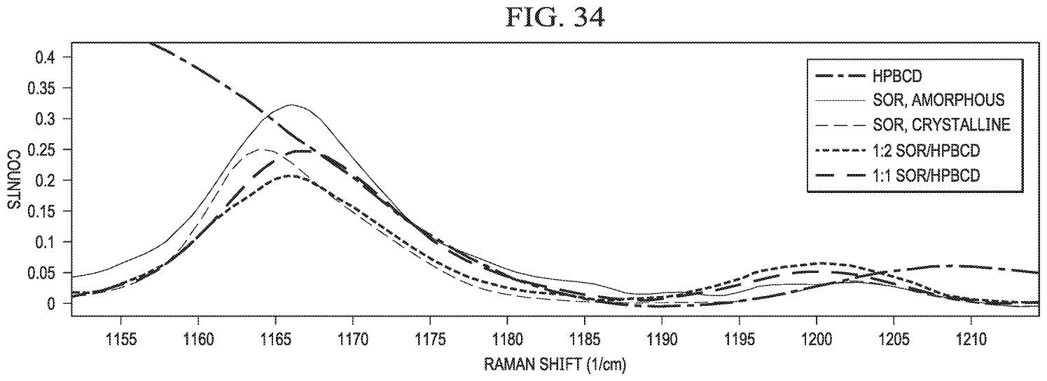

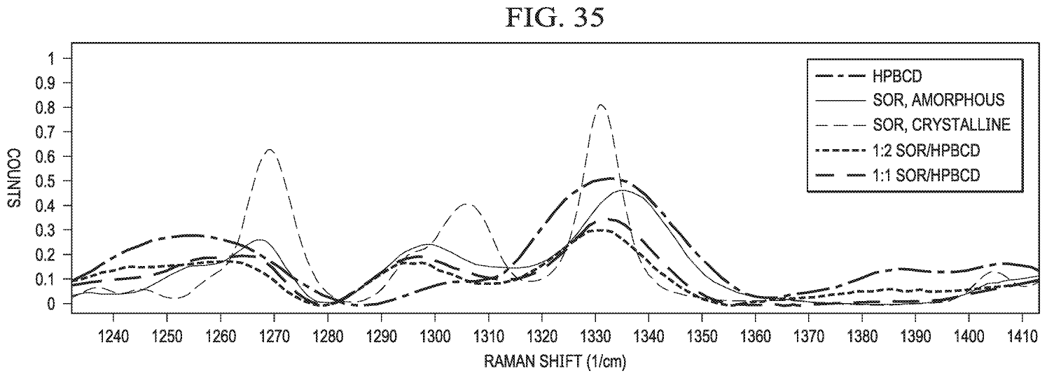

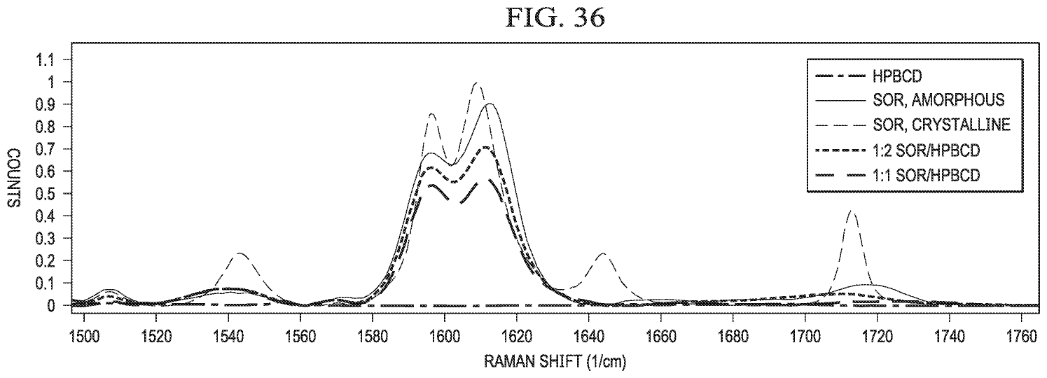

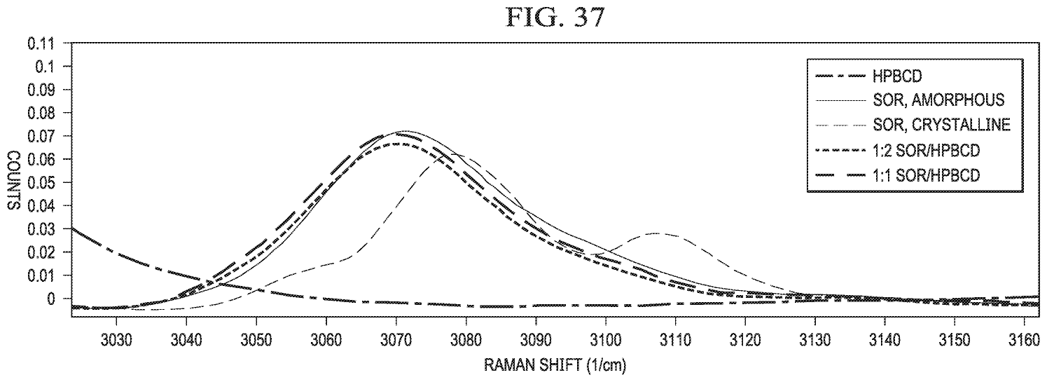

Comparing results across columns of the crystalline SOR, HPBCD, amorphous SOR, and KPCs of 1:2 SOR/HPBCD and 1:1 SOR/HPBCD, for each of Rows 1-16 indicated in FIG. 26, the following observations are made. For data shown in Row 1, the KPCs shift to higher wavenumbers. Details of the spectral shift of Row 1 are shown in FIG. 27. For data shown in Row 2, the KPC peaks appear to have shifted from approximately 417 cm⁻¹. For data shown in Row 3, the KPC peaks have broadened and shifted to lower wavenumber. Details of the spectral shift of Rows 2 and 3 are shown in FIG. 28. For data shown in Row 4, 1:1 KPC appears to have some amorphous SOR content. Details of the spectral shift of Row 4 are shown in FIG. 29. For data shown in Rows 5 and 6, KPC peaks have shifted to a higher wavenumber. Details of the spectral shift of Rows 5 and 6 are shown in FIG. 30. For data shown in Row 7, KPC and amorphous peaks are significantly different from crystalline SOR peaks. For data shown in Row 8, crystalline SOR peaks at approximately 918 cm⁻¹ and 928 cm⁻¹ appear shifted to higher wavenumbers. Details of the spectral shift of Rows 7 and 8 are shown in FIG. 31. For data shown in Row 9, KPC peaks and amorphous SOR peaks have shifted to higher wavenumber. Details of the spectral shift of Row 9 are shown in FIG. 32. For data shown in Row 10, KPC and amorphous peaks have broadened and shifted to higher wavenumber. Details of the spectral shift of Row 10 are shown in FIG. 33. For data shown in Row 11, KPC peaks have broadened and shifted to lower wavenumber. Details of the spectral shift of Row 11 are shown in FIG. 34. For data shown in Row 12, KPC peaks appear to have shifted to lower wavenumber. For data shown in Row 13, KPC peaks have decreased in intensity and shifted to lower wavenumber compared to amorphous SOR peak. Details of the spectral shift of Rows 12 and 13 are shown in FIG. 35. For data shown in Row 14, KPC peaks have decreased in intensity and shifted to lower wavenumber compared to amorphous SOR peaks. For data shown in Row 15, KPC peaks appear broadened compared to amorphous SOR. Details of the spectral shift of Rows 14 and 15 are shown in FIG. 36. For data shown in Row 16, KPC peaks have decreased in intensity and shifted to lower wavenumber compared to amorphous SOR peak. Details of the spectral shift of Row 16 are shown in FIG. 37.

Conclusions. In summary, modulated differential scanning calorimetry (mDSC), heating studies, and X-ray diffraction (XRD) results support the conclusion that the SOR appears fully included in the relatively hydrophobic cavity of in HPBCD when the SOR/HPBCD molar ratios are 1:1 or 1:2 in KPCs. Thus, inclusion complexes of SOR API with HPBCD cyclic oligomer were formed.

Many of the sorafenib peaks appear to have slightly different Raman shifts in the KPCs. It is possible that both 1:2 and 1:1 SOR/HPBCD complex stoichiometries form. Alternatively, the 1:1 may contain some unincluded, amorphous sorafenib at concentrations low enough to prevent recrystallization upon heating.

Additional evaluations of Raman spectral assignments may be used for elucidating which sorafenib functional groups are interacting with the HPBCD cavity. These additional analyses are expected to be in agreement with the results and conclusions described in this Example.

### Example 4: Formulation of Inclusion Complexes of Rivaroxaban (RIV) and Hydroxypropyl β Cyclodextrin (HPBCD) Using Thermokinetic Compounding

This example describes thermokinetic compounding formulations of Rivaroxaban (RIV, as the API) together with hydroxypropyl β cyclodextrin (HPBCD) and analyses of the resulting formulated compositions to show formation of inclusion complexes.

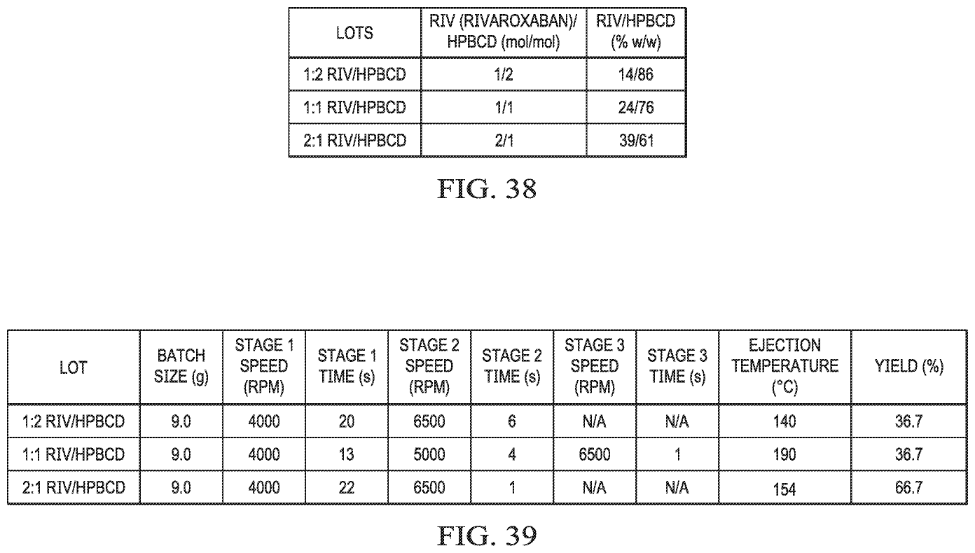

Rivaroxaban (RIV) and hydroxypropyl β cyclodextrin (HPBCD) composition lots were prepared as shown in FIG. 38. Rivaroxaban and HPBCD raw powders were blended by hand in a suitable container for feeding into the formulator. The lots were processed by thermokinetic compounding as shown in FIG. 39 to provide KinetiSol®-processed composition (KPC) lots. After thermokinetic compounding, lot samples were processed in an IKA Tube Mill 100 at 25,000 rpm for 30 seconds to reduce particle size and then passed through a #60 Mesh (250 μm).

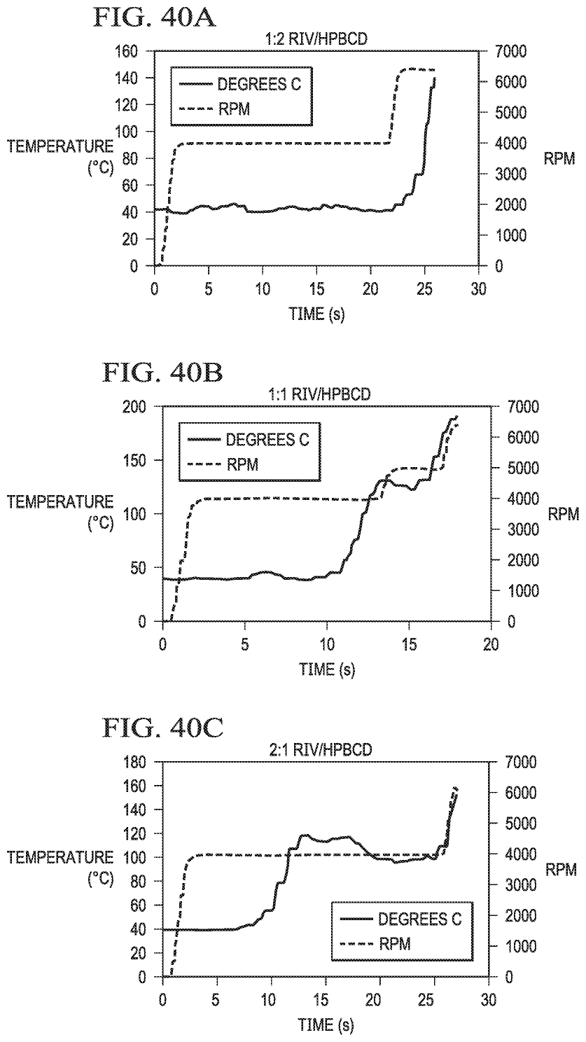

Thermokinetic compounding profiles. Results of thermokinetic compounding profiles for KinetiSol®-processed composition (KPC) lots 1:2 RIV/HPBCD, 1:1 RIV/HPBCD and 2:1 RIV/HPBCD are shown in FIG. 40A, FIG. 40B and FIG. 40C, respectively.

The 1:2 RIV/HPBCD was subsequently shown to recrystallize upon heating to 200° C. (see below). The ejection temperature of the 1:2 formulation was set to 140° C. It is expected that fully included 1:2 RIV/HPBCD may be formed by increasing the ejection temperature to at least 190° C.

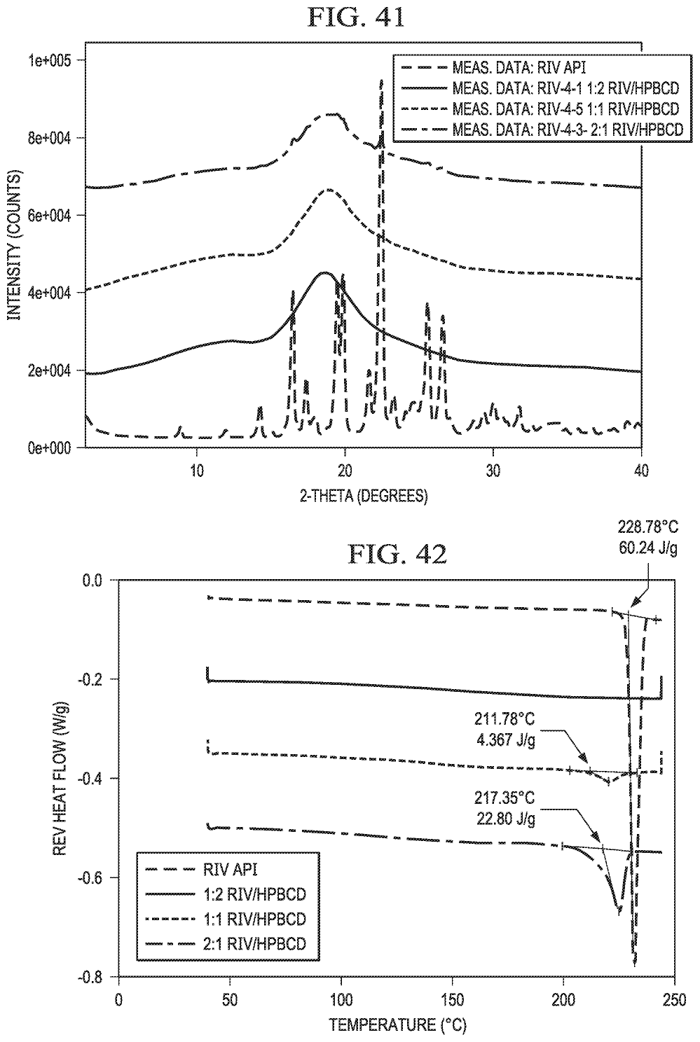

X-ray diffraction (XRD). X-ray diffraction (XRD) results for lots 1:2 RIV/HPBCD, 1:1 RIV/HPBCD and 2:1 RIV/HPBCD, as well as crystalline RIV API are shown in FIG. 41. FIG. 41 shows that 1:2 and 1:1 RIV/HPBCD were amorphous by XRD, but subsequent heating studies (see below) showed that RIVA was available for crystallization in both the 1:2 and 1:1 KPCs as well as the 2:1 KPC.

Modulated differential scanning calorimetry (mDSC). Modulated differential scanning calorimetry (mDSC) results for lots 1:2 RIV/HPBCD, 1:1 RIV/HPBCD and 2:1 RIV/HPBCD, as well as crystalline RIV API are shown in FIG. 42. As shown in FIG. 42, at the 1:2 RIV/HPBCD ratios, the RIV appears to be completely included in the HPBCD. In comparison to the heating studies (see below), mDSC appears to be less sensitive to detect unincluded RIV.

Heating Studies: X-Ray Diffraction (XRD) of Heated RIV-HPBCD Samples.

Lots 1:2 RIV/HPBCD, 1:1 RIV/HPBCD and 2:1 RIV/HPBCD KPCs were heated to 200° C. for 6 hours. This is about 30° C. below the temperature of API melting onset. The heated KPC's were allowed to cool and analyzed by XRD.

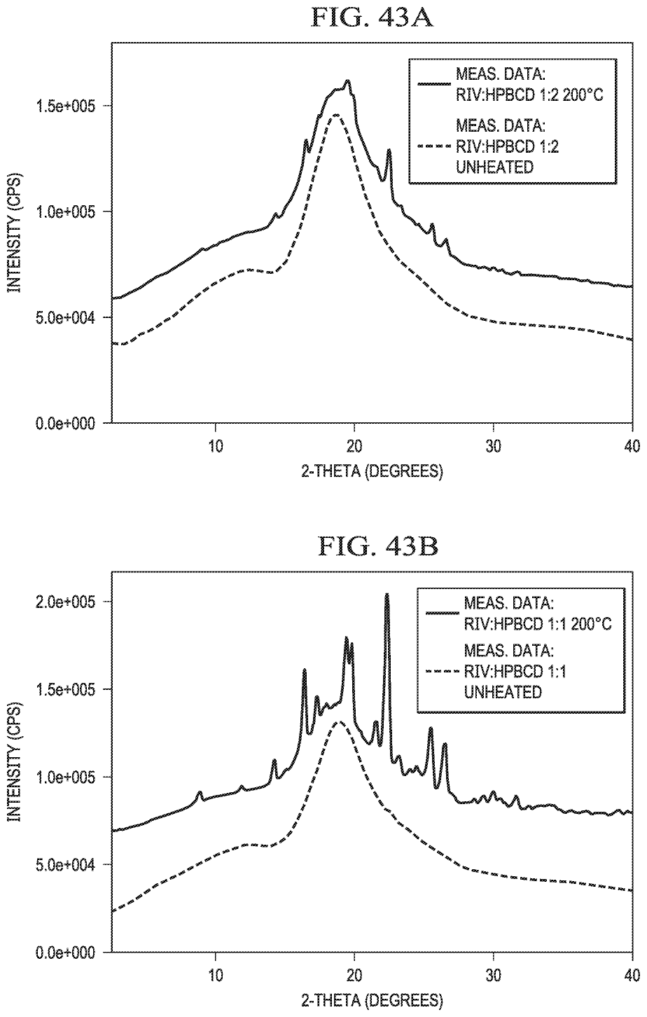

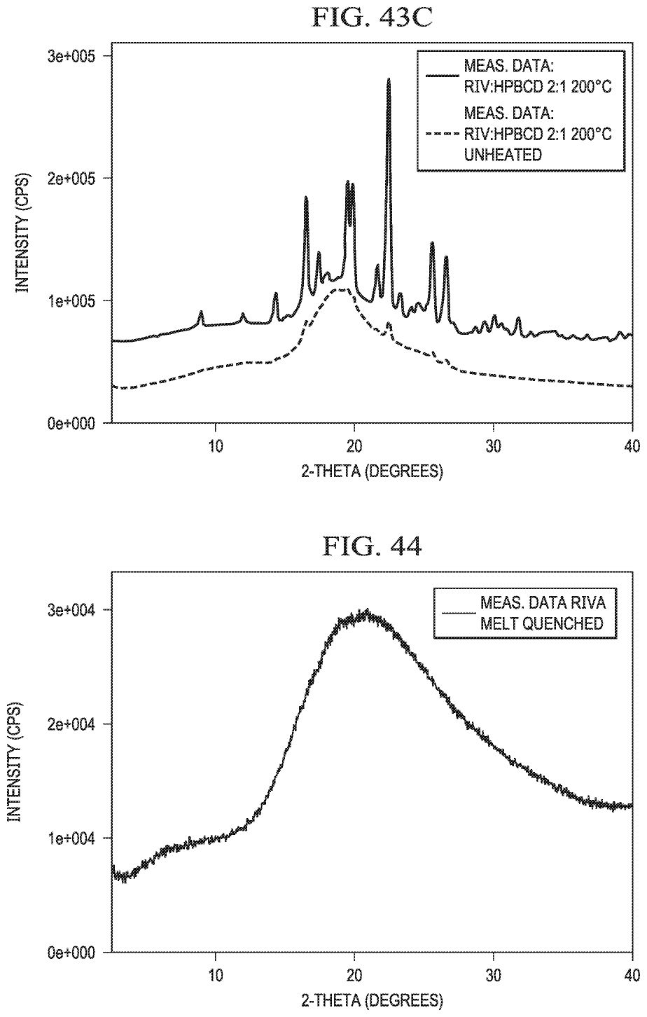

As shown in FIG. 43A, the 1:2 RIV/HPBCD KPC showed some recrystallization upon heating to 200° C. for 6 hours, indicating the presence of a small amount of unincluded rivaroxaban in the KPC.

As shown in FIG. 43B, the 1:1 RIV/HPBCD KPC showed substantial crystallization upon heating to 200° C. for 6 hours, indicating the presence of a large amount of unincluded, amorphous rivaroxaban in the KPC.

As shown in FIG. 43C, the 2:1 RIV/HPBCD KPC showed substantial crystallization upon heating to 200° C. for 6 hours, indicating the presence of a large amount of unincluded, amorphous rivaroxaban in the KPC.

Amorphous RIV. Amorphous RIV was prepared by melt-quenching for Raman spectral comparison with crystalline and HPBCD-included RIV. Approximately 600 mg of RIV were placed in a 20-mL scintillation vial. The vial was heated until the RIV had completely melted. The vial containing the melt was submerged in liquid nitrogen to solidify the RIV in its amorphous form. FIG. 44 is a graph reporting example RIV results for amorphous RIV that was prepared by melt-quenching.

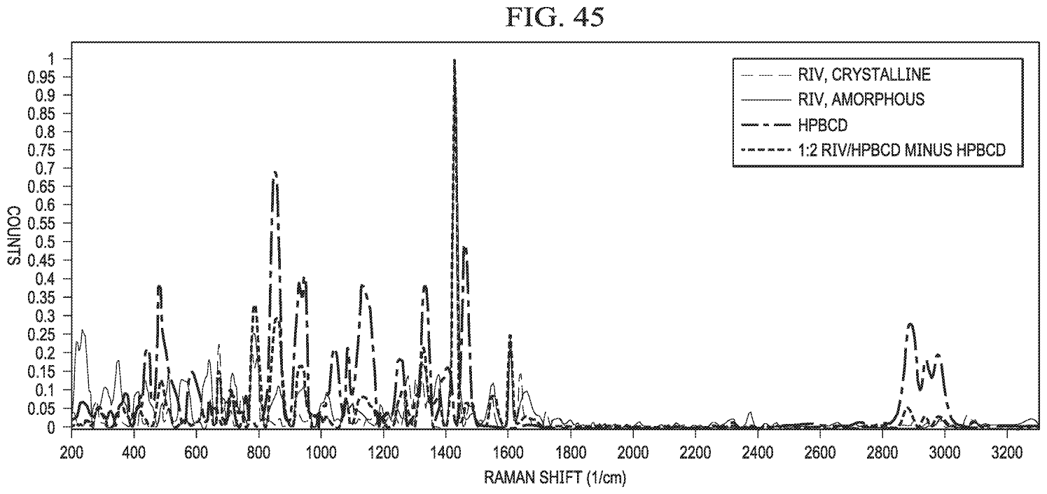

Raman spectroscopy. Raman spectroscopy was used to analyze crystalline RIV, HPBCD, amorphous RIV, and KPC of 1:2 RIV/HPBCD. FIG. 45 is a graph reporting example Raman spectra of crystalline RIV, HPBCD, amorphous RIV, and KPC of 1:2 RIV/HPBCD. Spectra shown are averages of three acquisitions. Spectral baseline corrections have been applied. The HPBCD spectrum was subtracted from that of the RIV/HPBCD KPCs. Interpretation of the Raman spectroscopy data is made in view of the X-ray diffraction (XRD) of heated RIV-HPBCD samples suggesting that 1:2 RIV/HPBCD KPC may contain a portion of amorphous, unincluded RIV.

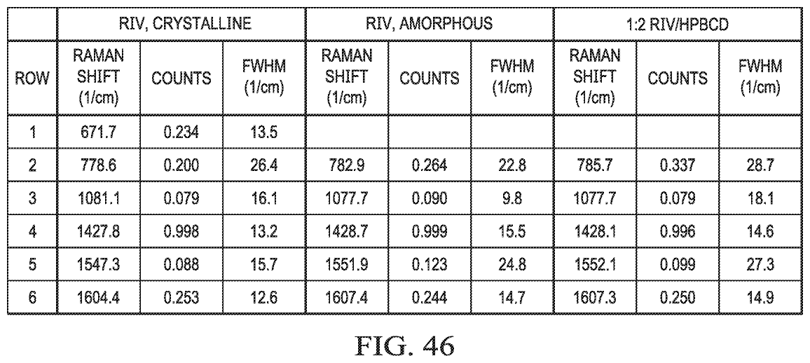

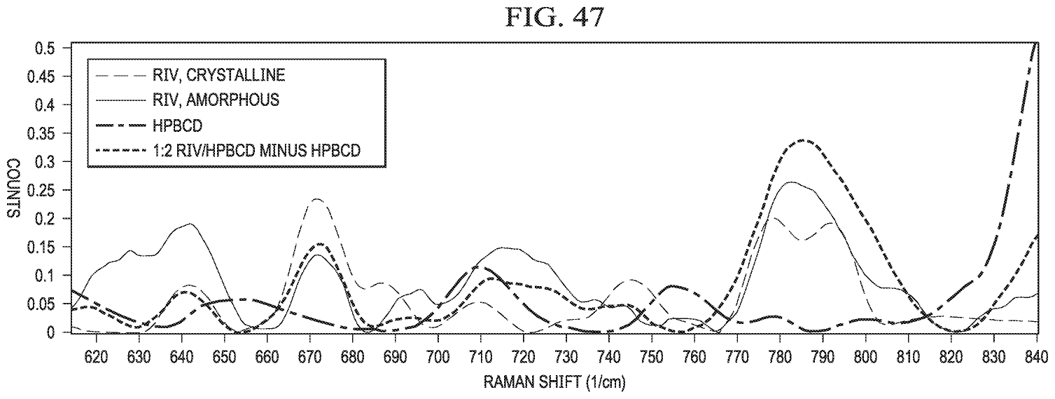

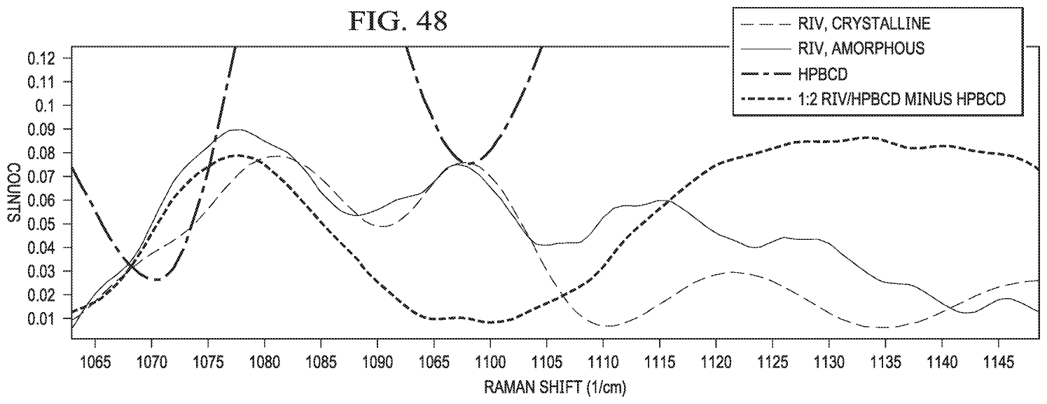

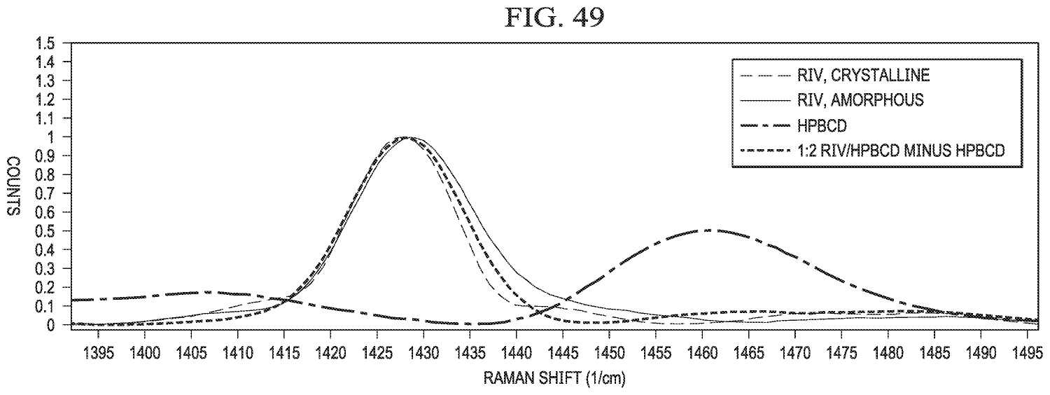

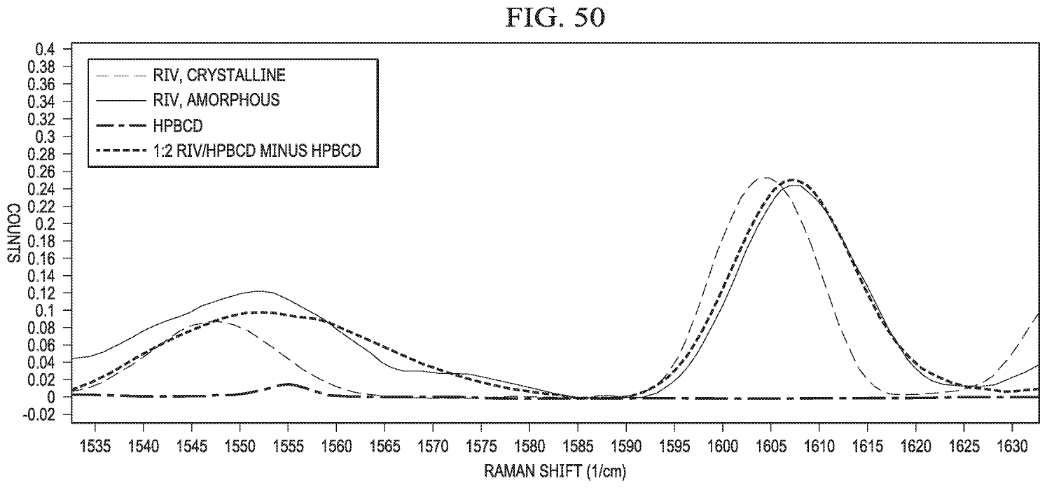

FIG. 46 is a Table summarizing results of Raman spectral shift for crystalline RIV, HPBCD, amorphous RIV, and KPC of 1:2 RIV/HPBCD. Comparing results across columns of the crystalline RIV, HPBCD, amorphous RIV, and KPCs of 1:2 RIV/HPBCD, for each of Rows 1-6 indicated in FIG. 46, the following observations are made. For data shown in Row 1, the KPC peak resembles amorphous rather than crystalline. For data shown in Row 2, two crystalline peaks have converged into a single peak in amorphous and KPC. Details of the spectral shift of Rows 1 and 2 are shown in FIG. 47. For data shown in Row 3, KPC peak has disappeared at ˜1097 cm⁻¹. Details of the spectral shift of Row 3 are shown in FIG. 48. For data shown in Row 4, the KPC peak is located between crystalline RIV and amorphous RIV peaks. Details of the spectral shift of Row 4 are shown in FIG. 49. For data shown in Rows 5 and 6, the KPC and amorphous RIV peaks broader and shifted to lower wavenumber. Details of the spectral shift of Rows 5 and 6 are shown in FIG. 50.

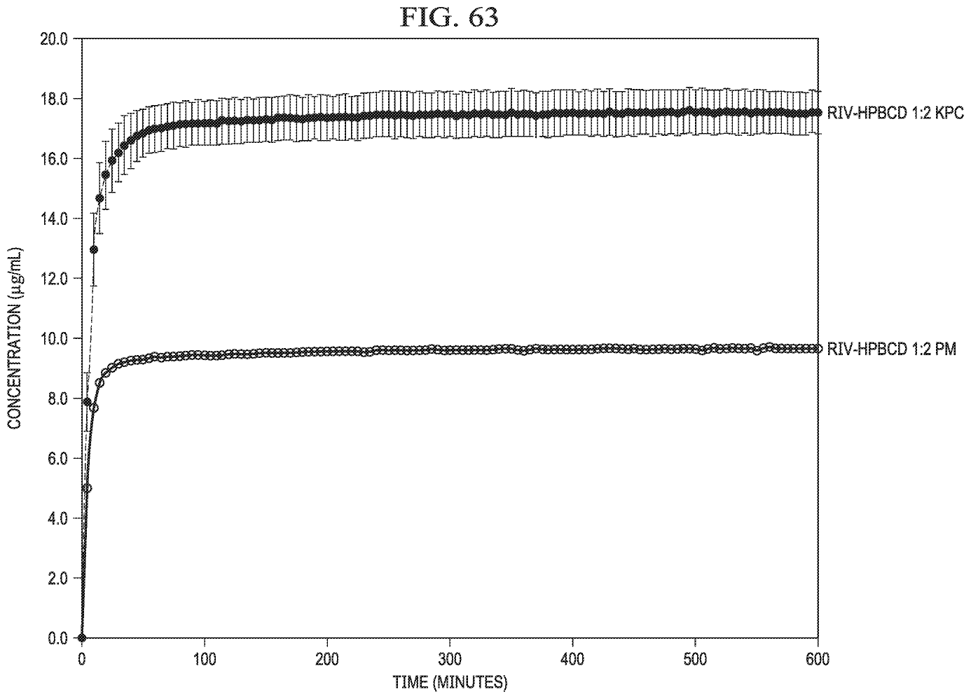

In vitro Dissolution Study. An in vitro dissolution method was used to analyze the dissolution of the 1:2 RIV/HPBCD physical mixture (PM, also referred to herein as “DST-36217-RIV-4-1-PM”) and a KPC thereof (also referred to herein as “DST-36217-RIV-4-1”). The USP II apparatus was used in Paddle test configuration. The aqueous solution test medium was 450 mL of FaSSIF media (Biorelevant). For dissolution analysis, samples equivalent to 20 mg of API for each of the 1:2 RIV:HPBCD) physical mixture (PM) and a KPC thereof (n=3 per sample) were added to the surface of the medium at the initiation of the test. The apparatus was set to 37° C. and a rotational speed of 75 rpm. UV spectra analysis (Pion Inc., Billerica, Mass., USA) was performed, with standardization in 8-28 μg/mL. Every 5 minutes over 10 hours, data were collected using 10 mm probe tips and analyzed by second derivative in Indigo software (Pion Inc., Billerica, Mass., USA). A wavelength range of 300-310 nm was used. As shown in FIG. 63, thermokinetic compounding of the 1:2 RIV:HPBCD formulation increased the AUDC observed from time 0 to time 10 hours by 1.8-fold as compared to the uncompounded physical mixture. As shown in FIG. 63, thermokinetic compounding of the 1:2 RIV:HPBCD formulation increased the Cₘₐₓ observed from 17.6 μg/mL in the KPC material as compared to 9.71 μg/mL in the uncompounded physical mixture.

Conclusions. In summary, modulated differential scanning calorimetry (mDSC), heating studies, and X-ray diffraction (XRD) results support the conclusion that formation of at least a 1:2 RIV/HPBCD complex should be attainable by thermokinetic compounding. Thus, inclusion complexes of RIV API with HPBCD cyclic oligomer were formed.

Approaches that are expected to achieve a KPC that contains 100% included rivaroxaban in HBPCD include (1) raising the ejection temperature from 140° C. in 20-degree increments, and/or (2) decreasing the RIV/HPBCD mole ratio, such that there is a further increased molar excess of HBPCD.

Additional evaluations of Raman spectral assignments may be used for elucidating which RIV functional groups are interacting with the HPBCD cavity. These additional analyses are expected to be in agreement with the results and conclusions described in this Example, and in additional methods of thermokinetic compounding formulation of an inclusion complex of RIV/HBPCD such as those in which, for example, include (1) raising the ejection temperature from 140° C. in 20-degree increments, and/or (2) decreasing the RIV/HPBCD mole ratio, such that there is a further increased molar excess of HBPCD.

Thermokinetic compounding of 1:2 RIV:HPBCD increased solubility of the RIV as compared to 1:2 RIV:HPBCD physical mixture (PM) in in vitro dissolution studies.

### Example 5: Formulation of Inclusion Complexes of Nintedanib (NIN) and Hydroxypropyl β Cyclodextrin (HPBCD) Using Thermokinetic Compounding

This example describes thermokinetic compounding formulations of Nintedanib (NIN, as the API) together with hydroxypropyl β cyclodextrin (HPBCD) and analyses of the resulting formulated compositions to show that inclusion complexes were formed.

NIN and HPBCD composition shots were prepared as shown in FIG. 54. The shots were prepared at three different molar ratios of NIN:HPBCD at 1:2 (lot 4), 1:1 (lot 2), and 2:1 (lot 3). NIN and HPBCD raw powders were blended by hand in a suitable container for feeding into the formulator. The lots were processed by thermokinetic compounding without added lubricant as shown in FIG. 54 to provide KinetiSol®-processed composition (KPC) lots.

After thermokinetic compounding, lot samples were processed in an IKA Tube Mill 100 at 25,000 rpm for 30 seconds to reduce particle size.

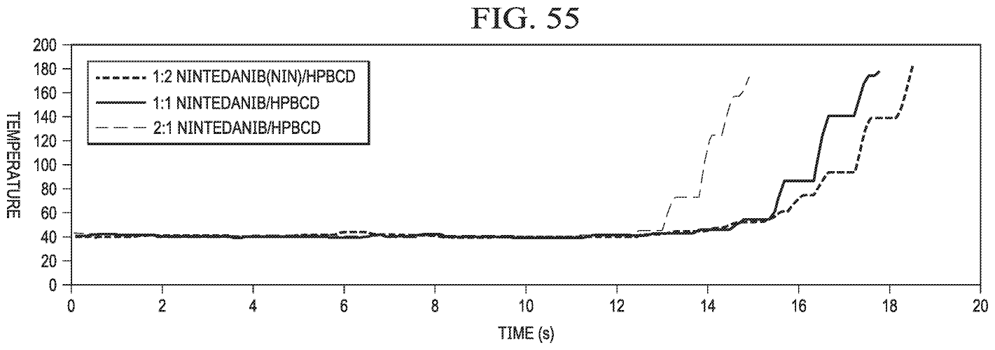

Thermokinetic compounding profiles. Results of thermokinetic compounding profiles for KinetiSol®-processed composition (KPC) lots NIN lot 2, 3 and 4 are shown in FIG. 55. All three shots were molten on ejection from the formulator.

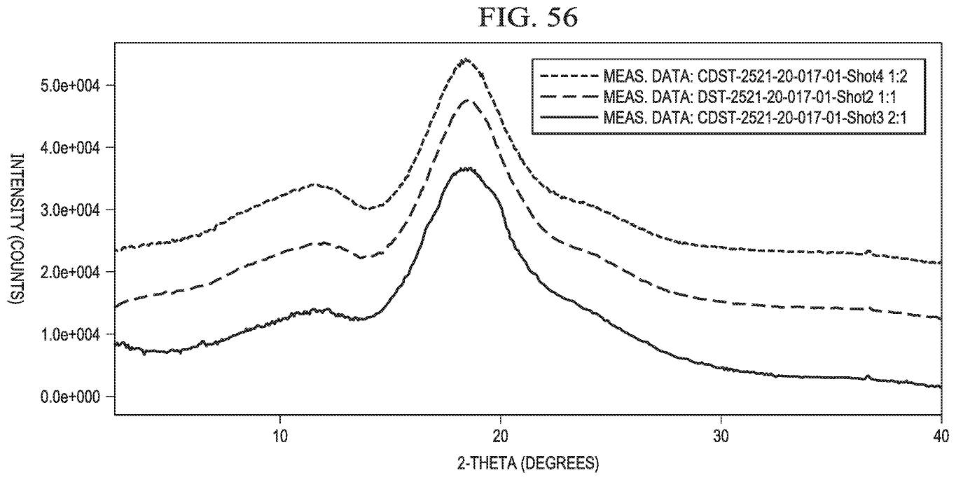

X-ray diffraction (XRD). X-ray diffraction (XRD) results for NIN lots 2, 3 and 4 are shown in FIG. 56. FIG. 56 shows that the 1:2 formulation (lot 4) and the 1:1 formulation (lot 2) appeared completely amorphous by XRD, while the 2:1 formulation (lot 3) appeared to be trace crystalline.

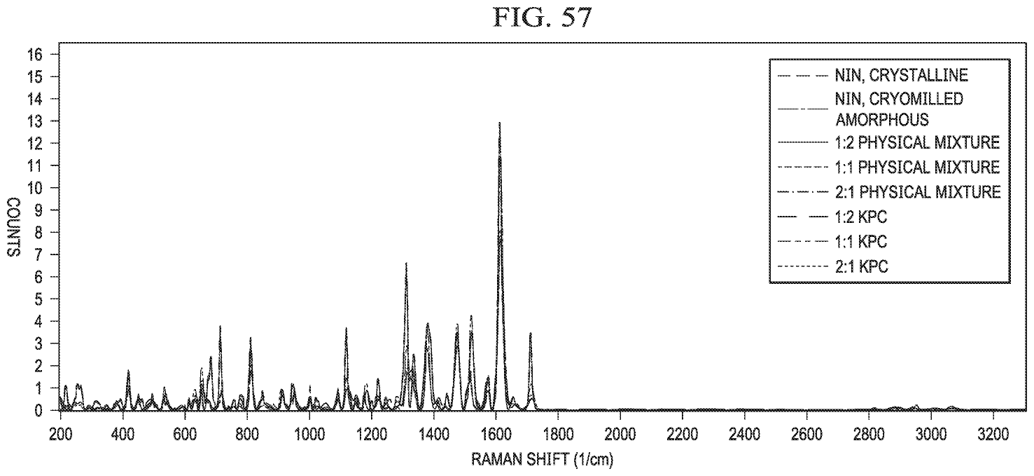

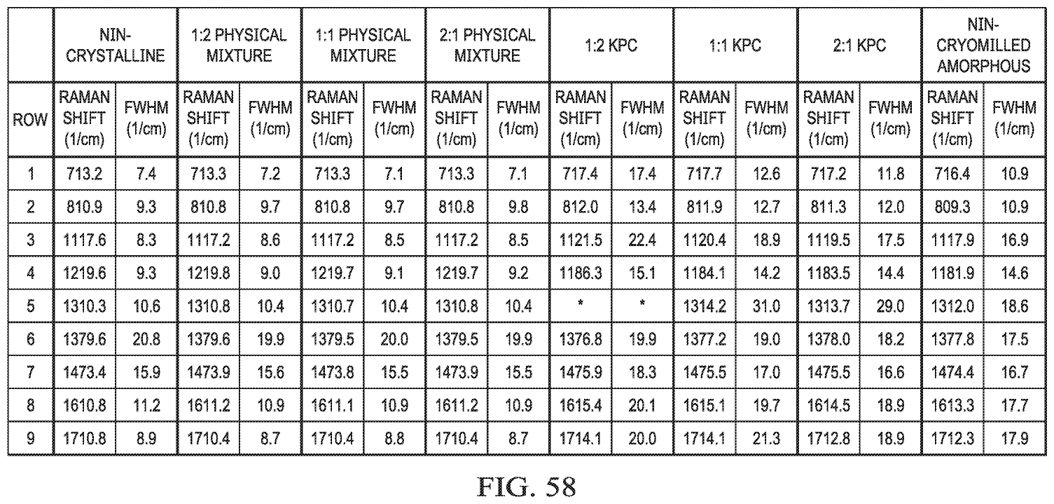

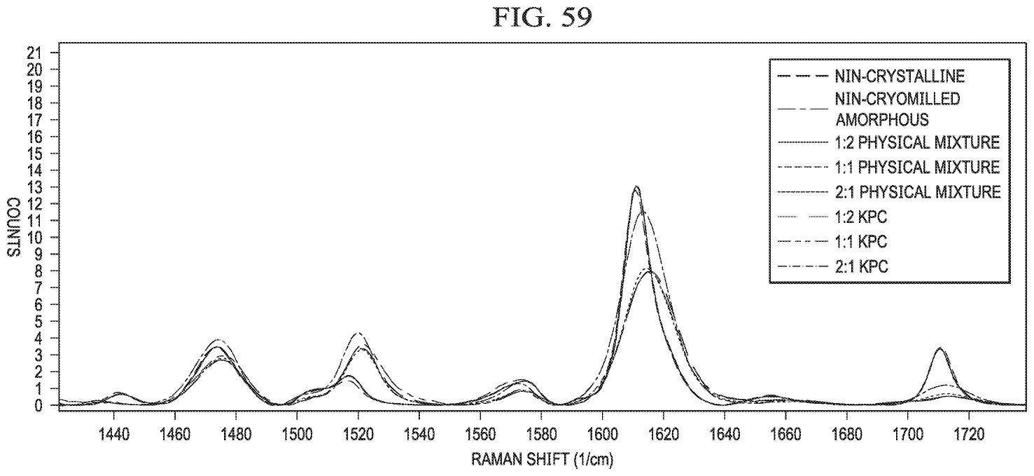

Raman spectroscopy. Raman spectroscopy was used to analyze crystalline NIN, cryo-milled amorphous NIN, and ITZ lots 4, 2 and 3 (NIN:HPBCD ratio of 1:2, 1:1, and 2:1 respectively) as physical mixtures, or as KPCs. FIG. 57 is a graph reporting example Raman spectra of these samples. Spectra shown are averages of three acquisitions. Spectral baseline corrections and peak normalizations have been applied. API peaks in which minimal interference from HPBC was observed were analyzed. FIG. 58 is a Table summarizing results of Raman spectral shift for these samples.

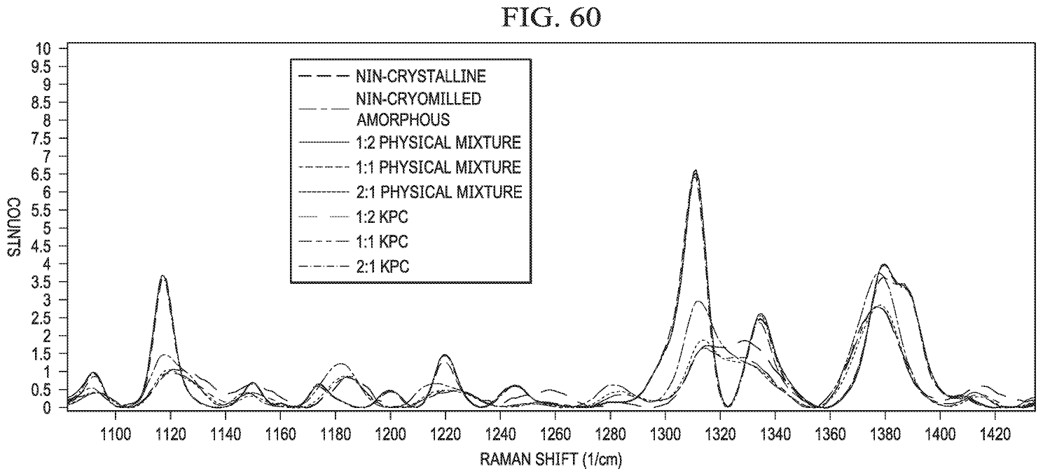

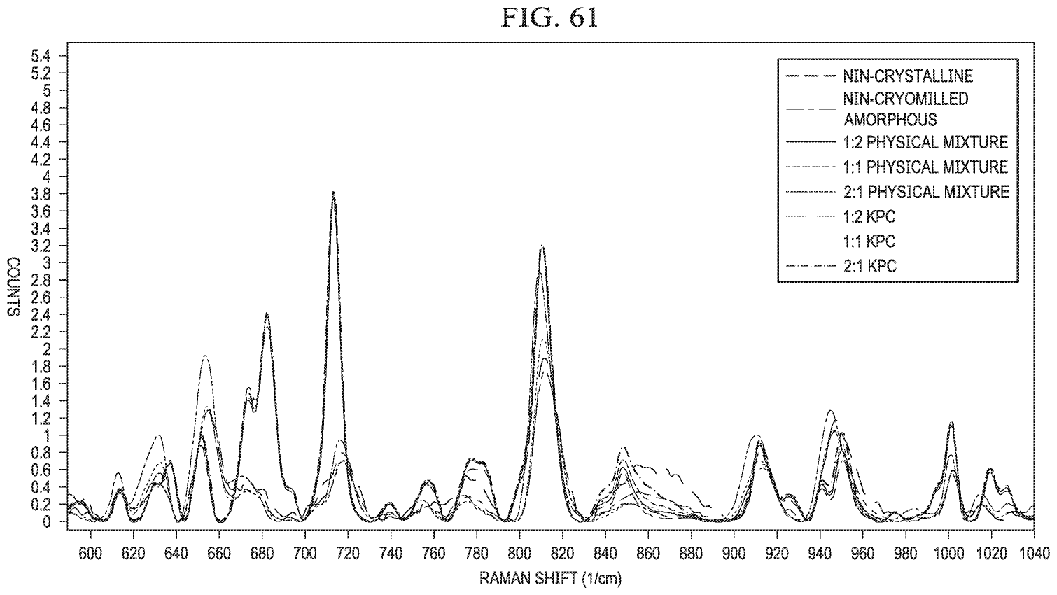

Comparing results across columns of the samples of crystalline NIN, NIN-HPBCD 1:2, 1:1, and 2:1 physical mixtures, NIN-HPBCD 1:2, 1:1, and 2:1 KPCs, and cryo-milled amorphous NIN, for each of Rows 1-9 indicated in FIG. 58, the following observations are made. 1:2 and 1:1 KPCs are very similar to one another in terms of Raman shifts and peak shapes. This suggests that the only difference in the two samples is the amount of uncomplexed HPBCD present, not the ratio of nintedanib:HPBCD complexed. 2:1 KPC shifts are generally shifted from the 1:2 and 1:1 KPCs slightly, consistent with amorphous, unincluded drug content. Details of the spectral shift of Rows 3-6 are shown in FIG. 60. Details of the spectral shift of Rows 1-2 are shown in FIG. 61.

The 1:2 and 1:1 NIN-HPBCD are very similar by Raman analysis, suggesting that the optimal ratio may be 1:1.

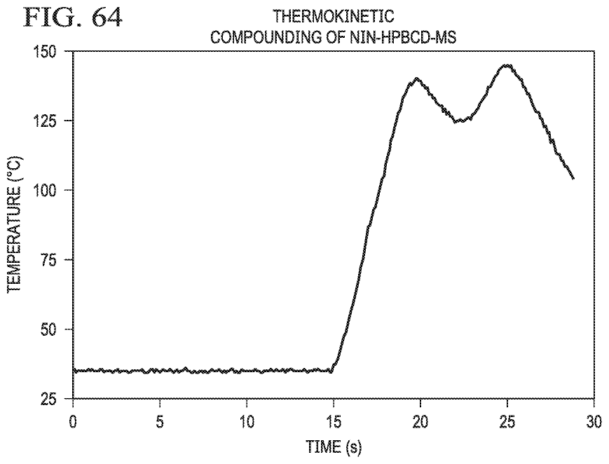

Formulation of 20% NIN, 79% HPBCD and 1% magnesium stearate (NIN-HPBCD-MS. A physical mixture (PM) of 20% NIN, 79% HPBCD and 1% magnesium stearate (NIN-HPBCD-MS) was prepared (referred to as “DST-2521.015.3”). The PM was thermokinetically compounded at 5,000 rpm for 20.8 seconds and at 6,000 rpm for 4.1 seconds. Example data of the temperature inside the thermokinetic mixer during thermokinetic compounding of a formulation of 20% NIN, 79% HPBCD and 1% magnesium stearate (NIN-HPBCD-MS) to form a KPC are shown in FIG. 64.

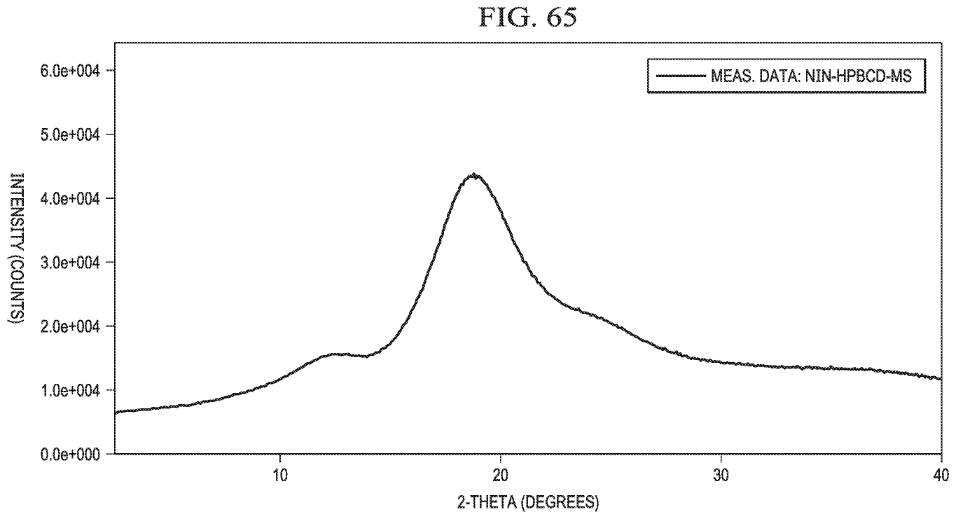

X-ray diffraction (XRD) of a KPC of NIN-HPBCD-MS. X-ray diffraction (XRD) results for the KPC of 20% NIN, 79% HPBCD and 1% magnesium stearate (NIN-HPBCD-MS) are shown in FIG. 65, which shows that the KPC of the NIN-HPBCD-MS formulation appeared completely amorphous by XRD.

HPLC analysis of a KPC of NIN-HPBCD-MS. A stability-indicating high-performance liquid chromatography (HPLC) method may be used for chemical analysis of nintedanib KPCs. A Dionex Ultimate 3000 HPLC system (ThermoFisher Scientific, Massachusetts, USA) may be used for reverse phase HPLC analysis. The HPLC column may be a Kinetex® C18, 150 mm×4.6 mm, 2.6 μm (Phenomenex, California, USA). Mobile phase A may be 0.1 M acetate buffer (pH 5) and mobile phase B may be degassed acetonitrile. A gradient profile with higher aqueous phase initially, followed by gradual increase in organic phase may be used. The flow rate may be 1.0 mL/min and the run time may be 35 min. The column may be held at 40° C., and the data may be collected at a single wavelength of 290 nm. Samples may be prepared at a nominal concentration of 0.5 mg/mL level with 1:1 acetonitrile:water as the standard/sample diluent. All samples may be filtered through 0.45 μm PVDF syringe filters (GE Healthcare Life-Sciences, Pennsylvania, USA), prior to analysis. Samples chromatography may be analyzed using Chromeleon™ software, version 7.2 (ThermoFisher Scientific, Massachusetts, USA).

Modulated Differential Scanning Calorimetry (mDSC) of a KPC of NIN-HPBCD-MS.

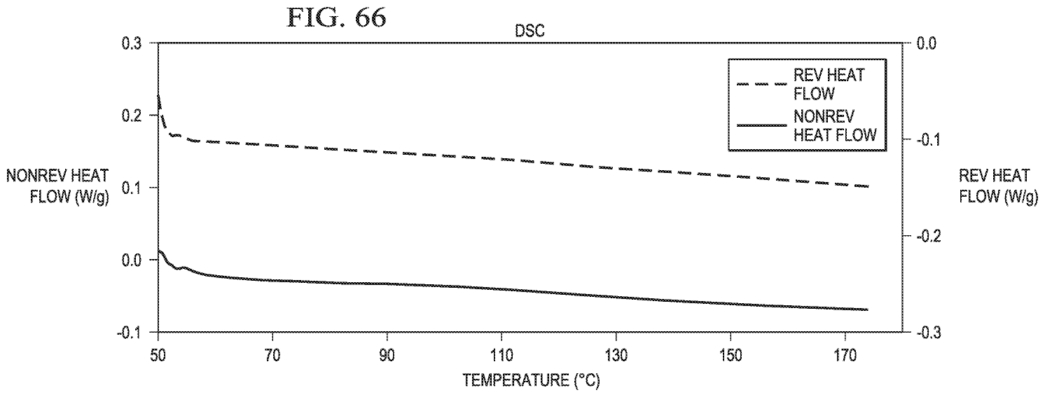

Modulated differential scanning calorimetry (mDSC) results from modulated differential scanning calorimetry (mDSC) analysis of a TKC of 20% NIN, 79% HPBCD and 1% magnesium stearate (NIN-HPBCD-MS) are shown in FIG. 66. The mDSC method was performed by equilibrating at 80° C., isothermal for 5 minutes, equilibrating at 50° C., then temperature was modulated+/−1.00° C. every 60 seconds, and ramped up 3.00° C. per minute up to 180.00° C. No significant glass temperature or melting event observed.

In vitro dissolution analysis of a physical mixture (PM) or a KPC of NIN-HPBCD-MS using a MicroFLUX™ apparatus. A MicroFLUX™ (Pion Inc., Billerica, Mass., USA) apparatus was used for in vitro dissolution analysis of a physical mixture (PM, indicated as “DST-2521.015.3”) of 20% NIN, 79% HPBCD and 1% magnesium stearate (NIN-HPBCD-MS), and a KPC thereof (indicated as “DST-2521.015.2”). The MicroFLUX™ apparatus has a lipid infused membrane separating a “donor” compartment, where a sample is placed for analysis, and a “receiver” compartment, for analysis of dissolved molecules that permeate the membrane separating the compartments. A physical mixture of 20% NIN, 79% HPBCD and 1% magnesium stearate (NIN-HPBCD-MS), or a KPC thereof, was added to the donor compartment containing 16 mL of pH 6.8 phosphate buffer with NaCl added, stirred at 500 rpm, at a concentration of 1 mg/mL API (NIN) content. The receiver compartment contained 16 mL of acceptor sink buffer (Pion proprietary media), stirred at 150 rpm. Duplicate samples were analyzed over a run time of 4 hours each. “FLUX” was calculated by AUPro software, version 6, (Pion Inc., Billerica, Mass., USA) over a range of 60 to 120 minutes. The donor compartment contents were analyzed by 0.5 mL sample withdrawal at 0.25, 0.5, 1, 2, and 4 hours, which were centrifuged at 13,000 rpm for 5 minutes, and the resulting supernatant was diluted 1:9 with 50/50 acetonitrile/water. Samples from donor and receiver compartments were analyzed by HPLC under the following conditions: 65/35 0.1 M acetate buffer, pH 5/acetonitrile, flow rate: 1.0 mL/min, run time: 12 min, with other parameters as described in Example 2.

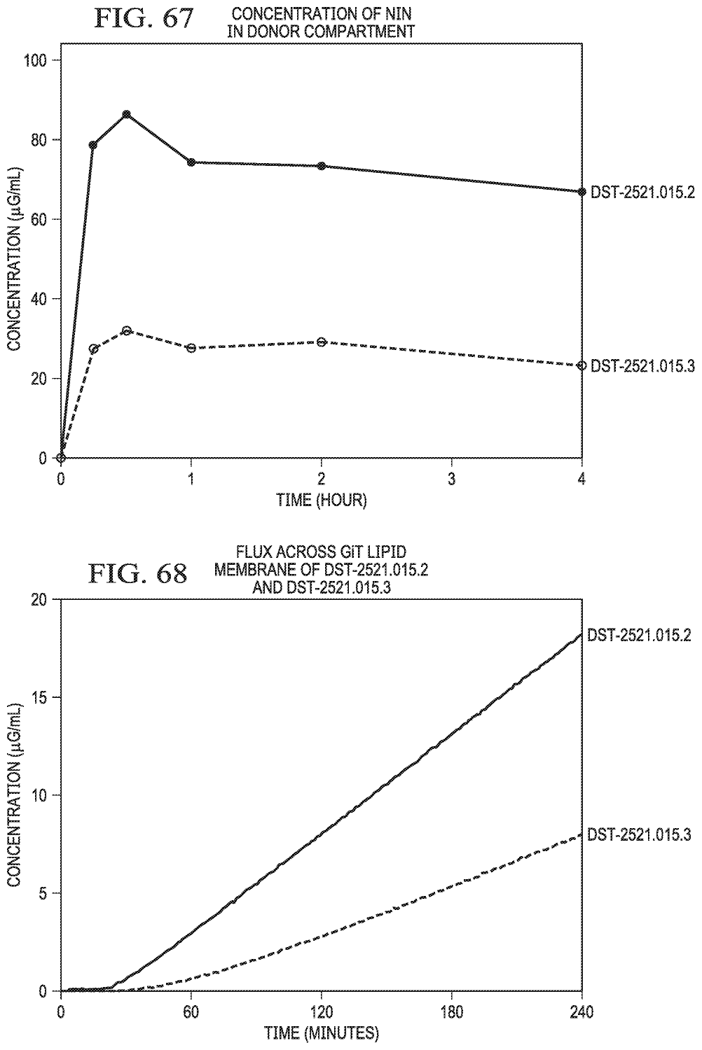

Results (average of duplicate samples) of the concentration of NIN dissolved from the physical mixture of 20% NIN, 79% HPBCD and 1% magnesium stearate (NIN-HPBCD-MS), or a KPC thereof, within the donor compartment of the MicroFLUX™ apparatus are shown in FIG. 67, and within the receiver compartment of the MicroFLUX™ apparatus are shown in FIG. 68. As shown in FIG. 67 and FIG. 68, the solubility of the KPC of NIN-HPBCD-MS was higher than the uncompounded physical mixture. As shown in FIG. 67, thermokinetic compounding of the KPC of NIN-HPBCD-MS increased the AUDC observed from time 0 to time 4 hours by 2.7-fold as compared to the uncompounded physical mixture. As shown in FIG. 67, thermokinetic compounding of the KPC of NIN-HPBCD-MS increased the Cₘₐₓ observed from 86.5 μg/mL in the KPC material as compared to 32.1 μg/mL in the uncompounded physical mixture. As shown in FIG. 68, thermokinetic compounding of the KPC of NIN-HPBCD-MS increased the FLUX observed from 8.2 μg*min/mL in the KPC material as compared to 4.2 μg*min/mL in the uncompounded physical mixture, or a 2.0-fold increase in FLUX.

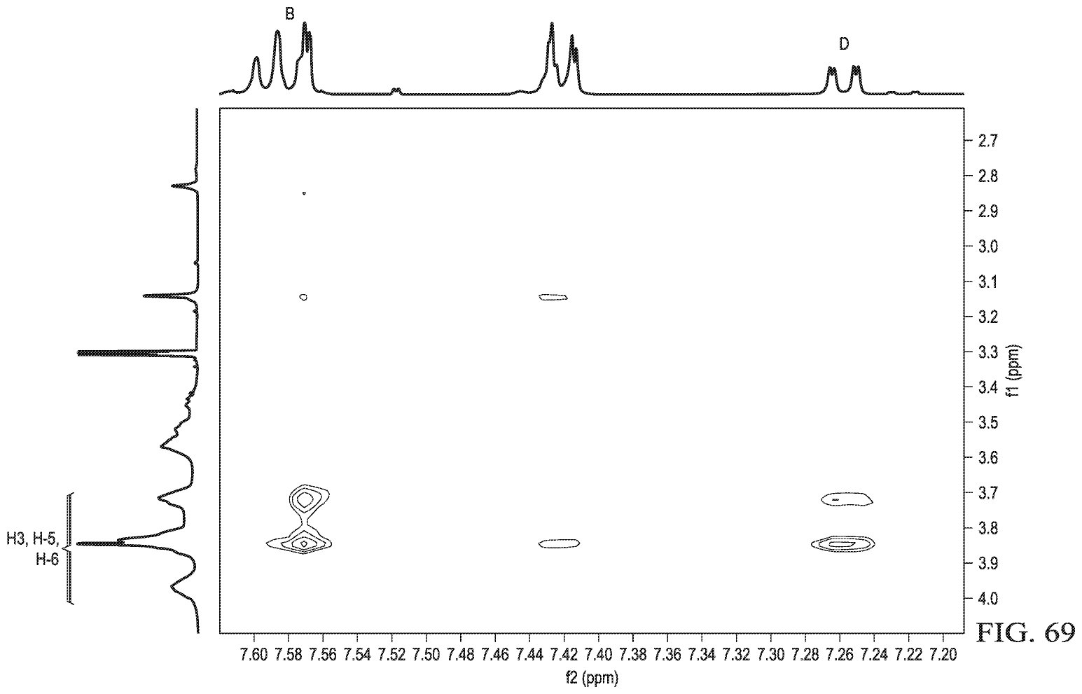

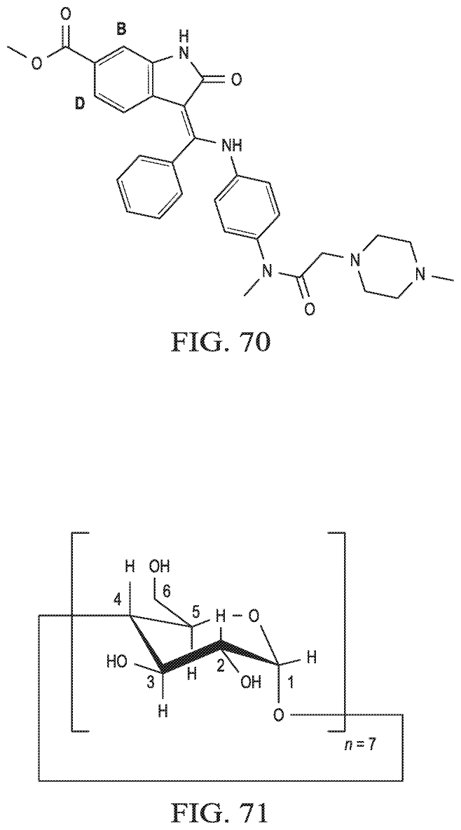

Nuclear Magnetic Resonance Spectroscopy. Two-dimensional Nuclear Overhauser Effect (2D NOESY) analysis was collected on a 1:1 Nintedanib/HPBCD complex in 70:30 CD3OD/D2O on an Agilent 600 MHz spectrometer. A total of 48 scans were collected. A relaxation delay of 1 s, a pulse width of 9.7 μs, and an acquisition time of 0.15 s were used. The cross peaks of the results shown in shown in FIG. 69 indicate that the nintedanib protons labeled B and D, as shown in FIG. 70, are in contact with the hydrophobic core (protons 3 and 5) of HPBCD. This supports the conclusion that an inclusion complex is formed. The aromatic protons of the oxindole of nintedanib are in contact with the HPBCD interior protons (H-5 and H-3, as shown in FIG. 71) in the inclusion complex.

The above disclosure contains various examples of inclusion complexes, pharmaceutical formulations including inclusion complexes, final solid dosage forms, methods of forming inclusion complexes and/or pharmaceutical formulations including inclusion complexes, and methods of administering pharmaceutical formulations including inclusion complexes. Aspects of these various examples may all be combined with one another, even if not expressly combined in the present disclosure, unless they are clearly mutually exclusive. For example, a specific inclusion complex or a pharmaceutical formulation including an inclusion complex may contain amounts of components identified more generally or it may be administered in any way described herein.

In addition, various example materials are discussed herein and are identified as examples, as suitable materials, and as materials included within a more generally-described type of material, for example by use of the term “including” or “such as.” All such terms are used without limitation, such that other materials falling within the same general type exemplified but not expressly identified may be used in the present disclosure as well.

The above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A pharmaceutical formulation comprising:
an inclusion complex;
an active pharmaceutical ingredient (API), or a pharmaceutically acceptable salt, ester, derivative, analog, prodrug, hydrate, or solvate thereof, or any combination thereof, wherein the API is not abiraterone or a pharmaceutically acceptable salt, ester, derivative, analog, prodrug, hydrate, or solvate thereof; and
a cyclic oligomer;
wherein at least a portion of the API is present in the inclusion complex with the cyclic oligomer; and wherein the pharmaceutical formulation is formed by a method comprising:
thermokinetically processing the API and the cyclic oligomer for less than 300 seconds to form an inclusion complex of the API and the cyclic oligomer.

2. The pharmaceutical formulation of claim 1, wherein at least 90%, 91%, 92%, 93%, 94%, 95%, 96%, 97%, 98%, 99%, or 99.9% of the API is present in the inclusion complex with the cyclic oligomer.

3. The pharmaceutical formulation of claim 1, wherein a molecule of the API, or the pharmaceutically acceptable salt, ester, derivative, analog, prodrug, hydrate, or solvate thereof, or any combination thereof, is sized to allow inclusion of at least 10%, 20%, 30%, 40%, 50%, 60%, 70%, 80%, 90% or 99.9% of the molecule of the API within a central cavity of the cyclic oligomer.

4. The pharmaceutical formulation of claim 1, wherein a molecule of the API, or the pharmaceutically acceptable salt, ester, derivative, analog, prodrug, hydrate, or solvate thereof, or any combinations thereof, has at least a minor axis diameter smaller than a diameter of a central cavity of the cyclic oligomer.

5. The pharmaceutical formulation of claim 4, wherein the diameter is a kinetic diameter.

6. The pharmaceutical formulation of claim 4, wherein the diameter of a central cavity of the cyclic oligomer is from 4 Å to 12 Å.

7. The pharmaceutical formulation of claim 4, wherein the diameter of a central cavity of the cyclic oligomer is up to 5, 6, 7, 8, 9, 10, 11, or 12 Å.

8. The pharmaceutical formulation of claim 1, wherein:
the cyclic oligomer is an α-cyclodextrin, or a derivative thereof; and
the API, or the pharmaceutically acceptable salt, ester, derivative, analog, prodrug, hydrate, or solvate thereof, or any combinations thereof, has a minor axis diameter up to 4.7-5.3 Å.

9. The pharmaceutical formulation of claim 1, wherein:
the cyclic oligomer is a β-cyclodextrin, or a derivative thereof; and
a molecule of the API, or the pharmaceutically acceptable salt, ester, derivative, analog, prodrug, hydrate, or solvate thereof, or any combinations thereof, has a minor axis diameter up to 6.0-6.5 Å.

10. The pharmaceutical formulation of claim 1, wherein:
the cyclic oligomer is a γ-cyclodextrin, or a derivative thereof, and
a molecule of the API, or the pharmaceutically acceptable salt, ester, derivative, analog, prodrug, hydrate, or solvate thereof, or any combinations thereof, has a minor axis diameter up to 7.5-8.3 Å.

11. The pharmaceutical formulation of claim 1, wherein:
the cyclic oligomer is a δ-cyclodextrin, or a derivative thereof; and
a molecule of the API, or the pharmaceutically acceptable salt, ester, derivative, analog, prodrug, hydrate, or solvate thereof, or any combinations thereof, has a minor axis diameter up to 10.3-11.2 Å.

12. The pharmaceutical formulation of claim 1, wherein the API is selected from itraconazole (ITZ), sorafenib (SOR), rivaroxaban (RIV), aceclofenac, alprostadil, AMG-330, amiodarone, aripiprazole, basedoxifene, benexate, betahistine, bexarotene, bicalutamide, BMS986231, bosutinib, Brexanolone, brivaracetam, budesonide, busulfan, cabozantinib, a cannabinoid, Carbamazepine, carfilzomib, cefotian-hexetil, cephalosporin, ceritinib, chloramphenicol, chlordiazepoxide, cisapride, cladribine, crizotinib, dasatinib, delafloxacin, dexamethasone, diclofenac sodium, enzalutamide, erlotinib, exemestane, fenofibrate, flunarizine, fosphenytoin, ganaxalone, gefitinib, glucagon, GNR-008, GS-5734, hydrocortisone, ibrutinib, idelalisib, imatinib, indomethacin, JPH-203, lamotrigine, lapatinib, lenalidomide, lenvatinib, letermovir, limaprost, LTP-03FA, maropitant, ME-344, Meloxicam, melphalan, merestinib, metronidazole, minoxidil, mitomycin, mitotane, ML-061, nilotinib, nimesulide, nintedanib, olaparib, omeprazole, OPC-108459, panobinostat, pazopanib, pevonedistat, piroxicam, pomalidomide, ponatinib, posaconazole, pramipexole, prexasertib, refocoxib, reproxalap, risperidone, RRR-dihydrotetrabenazine (RRR-DHTBZ), SAGE-689, sonidegib, sugammadex, sunitinib, TAK-020, telavancin, thalidomide, thioguanine, Thiomersal, tiaprofenic acid, topiramate, trametinib, tretinoin, vismodegib, Voriconazole, VTX-1463, VTX-2337, ziprasidone, a pharmaceutically acceptable salt, ester, derivative, analog, prodrug, hydrate, or solvate thereof, and any combinations thereof.

13. The pharmaceutical formulation of claim 1, wherein the API and the cyclic oligomer are present in the pharmaceutical formulation in a molar ratio of API:cyclic oligomer from 1:0.25 to 1:25.

14. The pharmaceutical formulation of claim 13, wherein the API and the cyclic oligomer are present in the pharmaceutical formulation in a molar ratio of API:cyclic oligomer from 1:1 to 1:3.

15. The pharmaceutical formulation of claim 1, wherein the API comprises less than 10%, 9%, 8%, 7%, 6%, 5%, 4%, 3%, 2%, 1%, or 0.1% crystalline API.

16. The pharmaceutical formulation of claim 1, wherein:
in response to heating the pharmaceutical formulation to a temperature up to 90% of the melting point of a crystalline form of the API, and
allowing the pharmaceutical formulation to cool to room temperature,
less than 10%, 9%, 8%, 7%, 6%, 5%, 4%, 3%, 2%, 1%, or 0.1% of the API is in crystalline form.

17. The pharmaceutical formulation of claim 16, wherein the less than 10%, 9%, 8%, 7%, 6%, 5%, 4%, 3%, 2%, 1%, or 0.1% of the API in crystalline form is determined by a method comprising X-ray diffraction (XRD).

18. The pharmaceutical formulation of claim 15, wherein the API comprises less than 10%, 9%, 8%, 7%, 6%, 5%, 4%, 3%, 2%, 1%, or 0.1% crystalline API as determined by a method comprising X-ray diffraction (XRD), modulated differential scanning calorimetry (mDSC), Raman spectroscopy, solid-state Nuclear Magnetic Resonance (NMR) spectroscopy, or any combination thereof.

19. The pharmaceutical formulation of claim 1, comprising 1% to 50% by weight of the API.

20. The pharmaceutical formulation of claim 1, comprising at least 10% by weight of the API.

21. The pharmaceutical formulation of claim 1, wherein the cyclic oligomer comprises a cyclic oligosaccharide or cyclic oligosaccharide derivative.

22. The pharmaceutical formulation of claim 21, wherein the cyclic oligosaccharide or cyclic oligosaccharide derivative comprises a cyclodextrin or a cyclodextrin derivative.

23. The pharmaceutical formulation of claim 22, wherein the cyclodextrin derivative comprises a hydroxy propyl β cyclodextrin.

24. The pharmaceutical formulation of claim 22, wherein the cyclodextrin derivative comprises a sodium (Na) sulfo-butyl ether β cyclodextrin.

25. The pharmaceutical formulation of claim 22, wherein the cyclodextrin derivative comprises a sulfobutylether functional group.

26. The pharmaceutical formulation of claim 22, wherein the cyclodextrin derivative comprises a methyl group.

27. The pharmaceutical formulation of claim 1, comprising 50% to 99% by weight of the cyclic oligomer.

28. The pharmaceutical formulation of claim 1, comprising at least 60% by weight of the cyclic oligomer.

29. The pharmaceutical formulation of claim 1, wherein the pharmaceutical formulation provides an increase in an Area Under the Drug Dissolution versus time Curve (AUDC), Cₘₐₓ, or both, as compared to a formulation of an equivalent amount of the API and the cyclic oligomer prepared without thermokinetically processing the API and the cyclic oligomer, when the pharmaceutical formulation is analyzed using an in vitro dissolution assay.

30. The pharmaceutical formulation of claim 29, wherein the AUDC, Cₘₐₓ, or both, is analyzed by a method comprising HPLC analysis, UV spectrophotometry, or both.

31. The pharmaceutical formulation of claim 1, wherein the thermokinetically processing the API and the cyclic oligomer is at a temperature less than or equal to 300° C.

32. A method of making a pharmaceutical formulation, the method comprising:
processing by thermokinetic compounding for less than 300 seconds:
(i) an API of claim 1; and
(ii) the cyclic oligomer of claim 1; and
(iii) to form the inclusion complex of claim 1.

33. The method of claim 32, wherein the processing by thermokinetic compounding does not cause substantial thermal degradation of the API.

34. The method of claim 32, wherein the processing by thermokinetic compounding does not cause substantial thermal degradation of the cyclic oligomer.

35. The method of claim 32, wherein the processing by thermokinetic compounding is solvent-free.
